(19) United States
(12) Patent Application Publication
(10) Pub. No.: US 2026/0062497 A1
(43) Pub. Date: Mar. 5, 2026

(54) Pharmaceutical Formulation and Dosing Regimen for the Treatment of Amyotrophic Lateral Sclerosis

(71) Applicant: AL-S Pharma AG, Schlieren (CH)

(72) Inventors: Marcel MAIER; Michael SALZMANN; Angela GENGE

(73) Assignee: AL-S Pharma AG, Schlieren (CH)

(21) Appl. No.: 19/106,573

(22) PCT Filed: Aug. 28, 2023
(86) PCT No.: PCT/EP2023/073571

§ 371 Date: Feb. 25, 2025

(30) Foreign Application Priority Data
Aug. 26, 2022 (EP) 22192430.1

(51) Int. Cl. C07K 16/40; A61K 9/00; A61K 9/08; A61K 31/167; A61K 31/192; A61K 31/4152; A61K 31/428; A61K 31/575; A61K 31/712; A61K 31/7125; A61K 39/00; A61K 45/06; A61K 47/20; A61K 47/22; A61K 47/26; A61P 25/28

(52) U.S. Cl. CPC C07K 16/40; A61K 9/0019; A61K 9/08; A61K 31/167; A61K 31/192; A61K 31/4152; A61K 31/428; A61K 31/575; A61K 31/712; A61K 31/7125; A61K 45/06; A61K 47/20; A61K 47/22; A61K 47/26; A61P 25/28; A61K 2039/505; A61K 2039/545

(57) ABSTRACT

Provided are pharmaceutical formulations and dosing regimen for antibody-based treatment of amyotrophic lateral sclerosis (ALS).

### FIELD OF THE INVENTION

The invention relates to the therapy of amyotrophic lateral sclerosis (ALS) and in particular to dosing regimen and formulations of antibodies that bind to misfolded forms of superoxide dismutase 1 (SOD1) for use in the treatment of ALS.

### BACKGROUND OF THE INVENTION

Amyotrophic lateral sclerosis (ALS) is a devastating neurodegenerative disease that primarily affects motor neurons of the motor cortex and spinal tract leading to severe disability and eventually death. About 90% of ALS cases have sporadic origin (sporadic ALS, sALS), whereas the remaining 10% develop the disease due to inherited gene mutations (familial ALS, fALS). The clinical manifestations of these two groups of ALS are indistinguishable, suggesting a convergence of varying pathways onto one unambiguous outcome, the degeneration of motor neurons and loss of muscles functions leading to variable amounts of weakness and spasticity in the limb, bulbar, and respiratory muscles. In most patients, limb onset, also referred to as spinal onset, begins with asymmetric painless weakness in a limb, but in approximately 20% of patients, weakness may be of bulbar onset manifesting as dysarthria or dysphagia. Bulbar onset patients have a poorer prognosis. Patients develop progressive disability limiting ambulation, communication, nourishment, and independence. Declining respiratory function can lead to respiratory insufficiency and failure which often is the cause for death, unless permanent mechanical ventilation is elected (Paulukonis et al., PLOS One 10 (2015), e0131965.2015, Poulin-Brière et al., Front. Neurosci. 15 (2021), 790114).

Although several efforts have been made to decipher the pathogenic mechanisms of ALS, its etiology remains elusive, with various mechanisms and cellular targets suggested. There is currently no cure for ALS. The only currently approved therapies for ALS worldwide are riluzole, Rilutek®, an oral medication that prolongs survival by 2-3 months, and edaravone, Radicava® (Miller et al., Cochrane Database Syst. Rev. 3 (2012), CD001447). These therapies have a limited impact on survival and disease progression, demonstrating a modest slowing of decline in the ALS functional rating scale (ALSFRS) in a subset of patients. Just recently, a combination of sodium phenylbutyrate and tauroursodeoxycholic acid (PB and TUDCA), Relyvrio™, and Tofersen, QALSODY™, have been approved for the treatment of ALS. However, none of the available therapies target wt-SOD1 and are directed to reverse SOD1 pathology.

Therefore, there is an urgent need for the therapy of ALS, preferably a therapeutic intervention which has a disease-modifying effect.

### SUMMARY OF THE INVENTION

The solution to the problem of providing a therapy of amyotrophic lateral sclerosis (ALS) is provided by the embodiments characterized in the claims and the description and illustrated in the Examples.

Thus, in a first aspect, the present invention generally relates to dosing regimen for the treatment of ALS with a recombinant antibody that binds to superoxide dismutase 1 (SOD1), i.e., anti-SOD1 antibody, wherein the antibody is administered to the subject, i.e., human patient at a high dose, preferably via intravenous administration. In a related aspect, the present invention relates to a drug formulation of said antibody, which is particularly suitable for that treatment regimen, in particular for intravenous administration. More specifically, the present invention relates to a recombinant antibody that selectively binds to misfolded and preferably aggregated forms of SOD1 for use in the treatment of ALS by administration of a dose of the antibody of 1000 mg to 10,000 mg, preferably of 1000 mg to 5000 mg, most preferably of about 2500 mg to a patient in need thereof. Administration is preferably performed once every 1 to 4 weeks, more preferably once every 2 to 4 weeks, most preferably once every 3 weeks. The drug formulation of the present invention is characterized by a stable liquid aqueous formulation of the anti-SOD1 antibody, which comprises the antibody at a concentration of about 10 to 50 mg/mL, preferably of about 20 mg/mL, in an L-histidine/L-histidine monohydrochloride buffer at a pH of about 6.0.

Different therapeutic strategies and targets have been tested in animal models with promising results, for example kinase inhibitors, gene-therapy, antisense oligonucleotides (ASO), antibody-based interventions and gene editing, using the CRISPR/Cas9 technology; see for review, e.g., Poulin-Brière et al., (2021), supra. In this context, therapeutic interventions based on passive immunization gained attention also for ALS and as summarized by Poulin-Brière et al., about 15 antibody targets are currently investigated.

The present invention is based on (i) the selection of an appropriate target and class of antibodies, (ii) development of a drug formulation wherein the antibody is stable, and which is tolerable for the patients even at high doses of the antibody, and (iii) establishing a treatment regimen that promises the best therapeutic prospects and at the same time is well tolerated by the patient, conditions that antibody-based approaches often fail to meet.

Regarding the first point, a recombinant human monoclonal antibody, specific to human misfolded SOD1 (mSOD1) has been used and investigated, designated AP-101, which has been originally disclosed in international application WO 2012/080518 A1 and further characterized in Maier et al., Sci. Transl. Med. 10. (2018) doi: 10.1126/scitranslmed.aah3924. Besides its disease-modifying properties, recent experiments suggest a high specificity of the anti-SOD1 antibody for mSOD1 in vivo as demonstrated by PET imaging.

Second, a pharmaceutical formulation of anti-SOD1 antibody has been developed, which provides for sufficient stability of the antibody and proved to be suitable for long term infusion of high doses of the antibody; see Examples 3 to 6.

Third, an appropriate dosing regimen has been established based on a multicenter, open-label, single ascending dose study (SAD) in patients with ALS using an open label oncology style 3+3 design (ClinicalTrials.gov Identifier: NCT03981536), wherein AP-101 was administered to patients via intravenous (IV) infusion over 1 hour at increasing dose levels of 100, 500 or 2500 mg, wherein certain time points for the read outs appeared to be essential to assess the safety, tolerability, and pharmacokinetics (PK) of antibody AP-101; see Examples 1 and 2. The potential for AP-101 to be administered at high doses in intervals of weeks without producing toxicity or unwanted effects was evaluated in a 1-month study in cynomolgus monkeys (Study 8384444) which consisted of 2 IV injections separated by 2 weeks. The monkey is a relevant toxicology species because AP-101 binds to human and monkey SOD-1; AP-101 does not bind to rodent SOD1. No AP-101-related effects on toxicology or safety pharmacology parameters occurred at doses up to and including 400 mg/kg (24,000 mg for a 60-kg person), the highest dose tested. In addition, the half-life of AP-101 from the monkey tox study was estimated 19.5 days. Based on the PK observed in cynomolgus monkeys and humans, PK profiles were utilized for calculation of human exposure multiples resulting in a dosing schedule of 2500 mg every three weeks, preferably with a 500 mg loading dose to help achieve the desired steady state more rapidly; see Table 3 in Example 2.

Accordingly, in one aspect, the present invention relates to a dosing regimen in the treatment of ALS, i.e., a recombinant antibody that selectively binds to misfolded and preferably aggregated forms of SOD1 for use in the treatment of ALS by administration of a dose of the antibody of about 2500 mg to a subject in need thereof, preferably once every 3 weeks. In a preferred embodiment, the administration is performed intravenously.

It was further found that the administration of a loading dose of 500 mg antibody one day before the first 2500 mg antibody dose is administered is advantageous. This kind of dosing scheme ensures that the subject to be treated reaches steady state as quickly as possible.

Accordingly, in one embodiment of the dosing regimen of the present invention the subject to be treated receives a loading dose of the antibody of about 500 mg, followed on the other day by a dose of the antibody of about 2500 mg prior to the administration of the antibody once every 3 weeks. This preferred dosing schedule is schematically depicted in FIG. 1.

In one embodiment, treatment will be performed for at least a 6-month period. In a particular preferred embodiment of the dosing regimen of the present invention, administration of the antibody at a dose of 2500 mg will take place once every 3 weeks for at least a 6-month period, preceded by the administration of a loading dose of the antibody of 500 mg one day before the administration of the first maintenance (effective) dose of the antibody of 2500 mg.

As mentioned above, different forms of ALS exist, i.e., sporadic amyotrophic lateral sclerosis (sALS) and familial amyotrophic lateral sclerosis (fALS). Accordingly, the subject to be treated may suffer from fALS or, alternatively, the subject to be treated suffers from sALS. Diagnosing of fALS and sALS can be performed with the highly sensitive immunoassay as disclosed in international application WO 2021/185961 A1, which comprises an anti-SOD1 antibody as capture antibody for determining the presence and level, respectively, of misfolded SOD1 (mSOD1) in a body fluid from a subject. The antibody is preferably the antibody as defined further below. Thus, in a preferred embodiment of the present invention, the subject to be treated has been diagnosed with the immunoassay as disclosed in WO 2021/185961 A1.

Preferably, the anti-SOD1 antibody to be used in the dosing regimen and drug formulation of the present invention is a human IgG and most preferably a human IgG1. In a preferred embodiment, the antibody is a human IgG1m3 allotype.

In a particular preferred embodiment, the anti-SOD1 antibody to be used in the dosing regimen and drug formulation of the present invention is AP-101 or an equivalent antibody derived from human antibody NI-204.12G7 as characterized in WO 2012/080518 A1 and Maier et al., Sci. Transl. Med. 10. (2018) doi: 10.1126/scitranslmed.aah3924. As explained further below, antibody AP-101 is a fully human IgG1m3 allotype antibody and thus comprises the human constant heavy chain (HC) amino acid sequence present in SEQ ID NO: 12 and a corresponding human constant light chain (LC), here lambda light chain as exemplified in SEQ ID NO: 13. As explained further below, IgG antibodies are made up as tetramers consisting of HC and two light LC chains linked by disulfide bridges.

Antibody AP-101 is preferably produced in Chinese hamster ovary (CHO)-K1 cells. CHO cells are the most widely used mammalian cells for the production of recombinant monoclonal antibodies due to their ability to perform post-translational modifications (PTMs) on the antibody molecules, which typically take place in the human body as well. Through genetic manipulation by mutagenesis, different CHO daughter cells with improved qualities have been established. Among those variants are CHO-K1, CHO-S, CHO-DXB11 and CHO-DG44.

Thus, in one embodiment, the antibody for use in accordance with the present invention is produced in CHO cells, preferably in a CHO-K1 cell line and is purified from the cell culture medium for further use.

As shown in Example 7, the major PTMs that have been identified in antibody AP-101 are the modification in the HC of glutamine at the N-terminus to pyro-glutamic acid, the loss of C-terminal lysine, and N-glycosylation. In this context, the N-glycosylation site was identified at position 303 (HC N303).

Thus, in one embodiment, the anti-SOD1 antibody for use in accordance with the present invention has lost the C-terminal lysine, i.e., the antibody has undergone C-terminal lysine clipping. In particular, the C-terminal lysine is chopped of the heavy chain of the antibody, preferably of each heavy chain of the antibody. In addition, or alternatively, the glutamine at the N-terminal is modified as pyro-glutamic acid, i.e., the antibody has undergone N-terminal glutaminyl cyclization. In addition, or alternatively, the antibody is glycosylated, in particular N-glycosylated. More particularly, the heavy chain of the antibody is glycosylated and even more particularly N303 of the heavy chain.

In a preferred embodiment, the anti-SOD1 antibody for use in accordance with the present invention lacks the C-terminal cysteine, has a modified glutamine at the N-terminal as pyro-glutamic acid and comprises at least one N-glycosylation site, or as used in a pharmaceutical formulation, preferably most of the antibody species present in the formulation have the mentioned modifications.

To assess the therapeutic effect of the antibody and the progression of ALS in human subjects, pharmacodynamic assessments can be performed as described in Example 2. In particular, the pharmacodynamic assessment includes the determination of changes in the level of total SOD1, misfolded SOD1 (mSOD1), phospho-neurofilament heavy chain (pNfH), and/or neurofilament light chain (NfL).

Accordingly, in one embodiment of the treatment in accordance with the present invention, the therapeutic effect of the antibody and progression of ALS is monitored by assessing one or more biomarkers, preferably any one of those mentioned above, and more preferably selected from the group consisting of phospho-neurofilament heavy chain (pNfH), neurofilament light chain (NfL) and misfolded SOD1 (mSOD1), or any combination thereof.

As mentioned above, riluzole and edaravone have been shown to prolong survival of ALS patients and thus, the present invention relates in one embodiment to the antibody for use in accordance with the present invention, wherein the subject to be treated receives riluzole or edaravone. In another embodiment of the antibody for use in accordance with the present invention the subject to be treated receives a combination of sodium phenylbutyrate and tauroursodeoxycholic acid (PB and TUDCA) or Tofersen.

In general, concomitant medication should be avoided. However, over-the-counter medications may be administered, e.g., acetaminophen for treatment of headaches or for the treatment of infusion reactions, if observed. Alternatively, or in addition, an antihistamine may be administered for the treatment of infusion reactions, if observed.

Accordingly, if headaches or infusion reactions are observed during the treatment in accordance with the present invention, in particular if infusion reactions are observed, acetaminophen or like analgesic and antipyretic agent may be administered. Typically, 500 to 1000 mg acetaminophen may be administered and preferably 30 to 60 minutes prior to the start of the administration of the antibody, in particular prior to the start of infusion of the antibody, i.e., for subsequent dose administrations. Administration of acetaminophen may be performed orally or, alternatively, intravenously.

In addition, or alternatively, if infusion reactions are observed during the treatment in accordance with the present invention, an antihistamine may be administered preferably 30 to 60 minutes prior to the start of the infusion of the antibody, i.e., for subsequent dose administrations. Also here, administration of the antihistamine can be performed orally and intravenously, respectively.

In one embodiment of the treatment in accordance with the present invention, the antihistamine and acetaminophen at the above-mentioned doses can both be administered via the mentioned administration routes if infusion reactions are observed.

In a particularly preferred embodiment of all embodiments of the dosing regimen of the present invention disclosed herein, the antibody is administered at a concentration of about 20 mg/mL, most preferably wherein the antibody is administered over a period of min. 60 and max. 120 minutes with an infusion rate of 500 mg (loading dose) and 2500 mg (maintenance or effective dose), respectively, in 60 minutes being preferred.

In the event that significant infusion reactions occur, the infusion rate should be lowered, preferably by 50% and preferably the infusion should be completed at that lower rate. Accordingly, in one embodiment, the administration of the antibody with the above-described infusion rate of 60 minutes is followed or interrupted with an about 50% lower infusion rate, i.e., 120 minutes.

As highly complex proteins, antibodies are susceptible to a variety of physical and chemical degradation pathways. Antibody aggregation easily occurs during storage in a liquid state leading to a loss of biological activity and increasing immunogenicity which in turn can cause serious adverse reactions like anaphylactic shock and other safety issues. To prevent the formation of aggregates that can cause undesirable immunogenicity or altered half-life, pharmaceutical antibody formulations usually contain one or more buffer(s) to maintain a given pH range. Since aggregation highly depends on antibody concentration and the pH of the formulation, the selection of a suitable buffer system is a crucial step towards a stable formulation. In addition, the desired formulation may be dependent from and should be suitable for and enable, respectively, the intended administration route so that the treatment is also well tolerated by the patients.

As illustrated in the Examples and proven in the SAD study (ClinicalTrials.gov Identifier: NCT03981536) as well as monkey study (Study 8384444), a buffer system could be established and drug formulation developed that provides both, appropriate stability of the antibody in aqueous solution and good tolerability when infused intravenously to patients over 60 to 120 minutes. In particular, for the preferred dosing regimen of the present invention, wherein the antibody is administered at a concentration of about 20 mg/mL, a formulation in an L-histidine/L-histidine monohydrochloride buffer at pH 6.0 has been found to be particularly suitable. A buffer system study was performed to determine the optimal buffer system for antibody formulation and based on a pH screening study, an L-histidine/L-histidine monohydrochloride buffer, pH 6.0 was chosen as the final buffer system, which ensured that a formulation comprising about 20 mg/ml of the antibody has long-term stability; see Examples 3 and 6 as well as Tables 14 to 17.

Furthermore, different types of excipients, including disaccharides (sucrose, trehalose and sorbitol), amino acids (L-arginine-hydrochloride and L-methionine) and salt (NaCl) were evaluated through excipients studies to further enhance the stability of the antibody formulation. The thermal stability, formation of insoluble aggregates and purity were monitored. Overall, sucrose and L-methionine with the concentration of 8% (w/v) and 0.1% (w/v), respectively, were chosen as the optimal excipients for AP-101 formulation, as they were shown to minimize the formation of low molecular weight and acidic species, thus retaining product purity; see Examples 3 and 6 as well as Tables 14 to 17.

In a surfactant type and strength screening study, four different polysorbate 80 (PS 80) concentrations (0.005%, 0.010%, 0.020% and 0.050% (w/v)) and two different poloxamer 188 concentrations (0.05% and 0.10% (w/v)) were tested. Number of sub-visible particles and purity were evaluated. A 0.02% (w/v) polysorbate 80 concentration was chosen as the surfactant strength as sub-visible particle formation was effectively suppressed and acceptable stability was shown; see Examples 3 and 6 as well as Tables 14 to 17.

Accordingly, in a further aspect, the present invention relates to a liquid aqueous pharmaceutical formulation of an anti-SOD1 antibody (drug formulation) that selectively binds to misfolded and/or aggregated forms of SOD1, wherein the formulation comprises the antibody at a concentration of about 10 to 50 mg/mL in a L-histidine/L-histidine monohydrochloride buffer at a pH of about 6.0±1, wherein the antibody remains stable at 5° C.±2° C. for at least 1 month, preferably for up to 24 months; at 25° C.±2° C. for at least 1 month, preferably for up to 6 months; and/or at 40° C.±2° C. for 1 week as shown in Tables 14, 15, 16, and 17.

In principle, the antibody can be any anti-SOD1 antibody which recognizes misfolded and preferably aggregated forms of SOD1, preferably preferentially or exclusively over the physiological form of SOD1. To avoid generation of “anti-drug antibodies” (ADA) by the patient, the antibody is preferably a human antibody, typically human IgG and most preferably a human IgG1. In a preferred embodiment, the antibody is a human IgGIm3 allotype. Hence, as mentioned hereinbefore, preferably the anti-SOD1 antibody to be used in the dosing regimen and drug formulation of the present invention is AP-101 or an equivalent antibody derived from human antibody NI-204.12G7 as characterized in WO 2012/080518 A1 and Maier et al., Sci. Transl. Med. 10. (2018) doi: 10.1126/scitranslmed.aah3924; see also infra.

As mentioned above, antibody AP-101 is preferably produced in Chinese hamster ovary (CHO)-K1 cells and thus, in one embodiment, the antibody for use in drug formulation of the present invention is produced in CHO cells, more particularly in a CHO-K1 cell line and is purified from the cell culture medium for formulation.

As also explained above, antibody AP-101 is a fully human IgG1m3 allotype antibody and preferably comprises a heavy chain, wherein the glutamine at the N-terminus is modified as pyro-glutamic acid, the C-terminal lysine is lost, and/or is glycosylated as explained in more detail above.

Thus, in one embodiment, the formulation of the present invention comprises an anti-SOD1 antibody as defined hereinbefore, wherein the antibody is composed of two heavy chains having SEQ ID NO: 12, and two light chains having SEQ ID: 13, and wherein in the heavy chain the glutamine at the N-terminus is modified as pyro-glutamic acid, the C-terminal lysine is lost, and the heavy chain is N-glycosylated.

As shown in Example 7, about 99% to 100% of the antibodies present in a sample of a typical antibody formulation have a N-terminal pyro-glutamic acid in the heavy chain and about 94% of the antibodies have a loss of the C-terminal lysine. Thus, in one embodiment, about 99% of the antibodies in the formulation of the present invention have a heavy chain wherein the N-terminal pyro-glutamic acid is modified from N-terminal glutamine and/or about 94% of the antibodies have a loss of the C-terminal lysine.

In one embodiment, the formulation of the present invention comprises the antibody at a concentration of 20±5 mg/mL, preferably 20 mg/mL.

In addition, or alternatively, the concentration of L-histidine/L-histidine monohydrochloride in the formulation of the present invention is 20±10 mM, preferably 20 mM.

Furthermore, in any one of the preceding embodiments, the formulation of the present invention preferably further comprises an antioxidant, preferably L-methionine at a concentration of 0.1%±0.05% (w/v), most preferably L-methionine at a concentration of 0.1% (w/v).

Preferably, in addition, or alternatively, any formulation of the present invention further comprises a tonicity modifier, preferably sucrose at a concentration of 8%±1% (w/v), most preferably sucrose at a concentration of 8% (w/v).

In a still further preferred embodiment, any formulation of the present invention further comprises a surfactant such as Polysorbate, preferably Polysorbate 80 at concentration of 0.02%±0.01 (w/v), most preferably Polysorbate 80 at a concentration of 0.02% (w/v).

The formulation of the present invention can have any of the above-mentioned composition provided that the antibody remains stable at 5° C.±2° C. for at least 1 month, preferably for up to 24 months; at 25° C.±2° C. for at least 1 month, preferably for up to 6 months; and/or at 40° C.±2° C. for 1 week.

In a particular preferred embodiment, the formulation of the present invention is substantially free of excipient(s) other than those specifically recited hereinbefore.

As mentioned above, the drug formulation of the present invention has been specifically developed for use in the dosing regimen of the present invention for the treatment of ALS. Accordingly, the dosing regimen of the present invention in the treatment of ALS is preferably performed with a drug formulation in an L-histidine/L-histidine monohydrochloride buffer of the present invention described above and disclosed in more detail below and in the Examples. In this context, the drug formulation which has been and is used in the clinical trials is the most preferred formulation of the present invention, which comprises or essentially consists of 20 mg/mL antibody, preferably AP-101 in 20 mM L-histidine/L-histidine monohydrochloride buffer, 8% (w/v), sucrose, 0.1% (w/v) L-methionine and 0.02% (w/v) polysorbate 80 at pH 6.0.

Furthermore, the compatibility of the antibody formulation with clinical in-use materials was evaluated. In particular, the compatibility with polyvinyl chloride IV bag, polypropylene syringe, PVC infusion set, etc. were assessed. Two concentrations 20 mg/mL and 5 mg/mL were assessed, and the data provided in Tables 11 to 13 in Example 5 indicate that the formulation is compatible with the clinical in-use materials evaluated.

The physical and chemical integrity of a biopharmaceutical must be maintained not only during long-term storage but also during administration. While some biopharmaceuticals are formulated and provided ready for clinical administration without further manipulation, many such products require varying degrees of handling by healthcare professionals. During handling and administration, the physical and chemical stability of the protein drug must be maintained.

When delivering a biopharmaceutical product via the intravenous route, several factors such as protein properties, formulation composition, concentration of the active pharmaceutical ingredient, choice of diluent, product contact surfaces, and/or infusion time and rate must be considered. The final critical factors assessed are environmental effects (e.g., area lighting and temperatures). The contact surface is of particular interest because proteins tend to adsorb at interfaces due to their amphiphilic nature. With the widespread use of a variety of plastic polymers in syringes and intravenous infusion containers and lines, the risk of protein loss by adsorption is substantial. Current pharmaceutical regulations require that intravenous-delivered medications be consistently within 10% of the nominal concentration; see Morar-Mitrica et al., MAbs. 7 (2015), 792-803 as well as references cited therein.

Thus, in an overall preferred embodiment, the formulation of the present invention is suitable for intravenous administration.

The present invention further relates to a medicament comprising the formulation of the present invention, wherein the medicament is for use in the treatment of ALS, most preferably for use in a dosing regimen of the present invention as defined above.

The present invention further relates to a pharmaceutical container comprising the formulation and the medicament, respectively, of the present invention. In a preferred embodiment, the container is a single-use glass vial to provide 100 mg of the antibody at a 20 mg/mL concentration.

In one embodiment, the container of the present invention, in particular the vial contains an approximate 10% volume overfill.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. The materials, methods, and examples are illustrative only and not intended to be limiting.

Other features and advantages of the invention will be apparent from the following detailed description and from the claims.

For the avoidance of any doubt it is emphasized that the expressions “in some embodiments”, “in a certain embodiments”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples for antibody AP-101 are intended to be generalized to any anti-SOD1 antibody and equivalents having substantially the same properties.

### BRIEF DESCRIPTION OF THE DRAWINGS

FIG. 1: Schematic illustration of one embodiment of the dosing schedule of the present invention

FIG. 2: Pharmacokinetic profile of antibody AP-101 in serum of ALS patients after administration of 100 mg, 500 mg, and 2500 mg, respectively, of the antibody.

### DETAILED DESCRIPTION OF THE INVENTION

The present invention relates to the embodiments as characterized in the claims, disclosed in the description and illustrated in the Examples and Figures further below, i.e., in a first aspect to dosing regimen for the treatment of amyotrophic lateral sclerosis (ALS) with a recombinant antibody that binds to SOD1 and in a related aspect to a drug formulation of the anti-SOD1 antibody, which is particularly suitable for that treatment regimen. In particular, the present invention relates to a recombinant antibody that selectively binds to misfolded and/or aggregated forms of SOD1 for use in the treatment of ALS in a dosing regimen by administration of a high dose of the antibody, e.g., of 1000 mg to 10,000 mg, preferably of 1000 mg to 5000 mg, most preferably of 2500 mg to a subject in need thereof, preferably once every 1 to 5 weeks, most preferably once every 3 weeks or by administration of another treatment regimen that delivers the antibody to the subject with substantially the same area under the curve. The drug formulation of the present invention is more specifically characterized by a stable liquid aqueous formulation of the anti-SOD1 antibody, which comprises the antibody at a concentration of about 10 to 50 mg/mL in an L-histidine/L-histidine monohydrochloride buffer at a pH of about 6.0. More specifically, the present invention relates to the embodiments as characterized in the claims, disclosed in the description and illustrated in the Examples and Figures further below.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-0-19852917-0.

Furthermore, unless stated otherwise, terms and expressions used herein in order to characterize the present invention are given in the definitions as provided in WO 2012/080518 A1, in particular in subsection “I. Definitions” at pages 10 to 30, the disclosure content of which is explicitly incorporated herein by reference. The same applies to the general embodiments disclosed in WO 2012/080518 A1 for antibodies, etc.

As used herein, the term “about,” refers to a value that is ±10% of a recited value; preferably ±5%.

As used herein, the term “subject” or “patient” is a human patient (e.g., a patient having ALS).

As used herein, the terms “subject” and “patient” are interchangeable.

In one embodiment, administration of the antibody in accordance with the present invention is performed according to a particular clinical dosing regimen (e.g., at a particular dose amount and/or according to a specific dosing schedule).

In one embodiment of the dosing regimen of the present invention, the anti-SOD1 antibody is administered at a fixed dose, i.e., that is fixed irrespective of the weight of the patient. The term “fixed dose” refers to a dose that is administered to a patient without regard for the weight or body surface area (BSA) of the patient. The fixed or flat dose is therefore, not provided as a mg/kg dose, but rather as an absolute amount of the antibody. In one embodiment, the antibody is administered at a fixed dose of 1000 mg to 10,000 mg, preferably of 1000 mg to 9000 mg, more preferably of 1000 mg to 7500 mg, more preferably of 1000 mg to 5000 mg, more preferably of 2000 mg to 3000 mg, and in particular of 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, or 10,000 mg, as well as any ranges in between, preferably of 2500 mg, to a subject in need thereof without regard to the subject's weight.

In one embodiment of the dosing regimen of the present invention, the anti-SOD1 antibody is administered at a milligram per kilogram (mg/kg) dose. In one embodiment, the antibody is administered at a dose of 17 mg/kg to 167 mg/kg, preferably of 17 mg/kg to 150 mg/kg, more preferably of 17 mg/kg to 125 mg/kg, more preferably of 17 mg/kg to 83 mg/kg, more preferably of 33 mg/kg to 50 mg/kg, and in particular of 17 mg/kg, 25 mg/kg, 33 mg/kg, 42 mg/kg, 50 mg/kg, 58 mg/kg, 67 mg/kg, 75 mg/kg, 83 mg/kg, 92 mg/kg, 100 mg/kg, 108 mg/kg, 117 mg/kg, 125 mg/kg, 133 mg/kg, 142 mg/kg, 150 mg/kg, 158 mg/kg, or 167 mg/kg, as well as any ranges in between, preferably of 42 mg/kg to a subject in need thereof.

In one embodiment of the dosing regimen of the present invention, administration of the antibody is performed once every 1 to 5 weeks, preferably once every 2 to 4 weeks, and in particular, once every week, once every 2 weeks, once every 3 weeks, once every 4 weeks, once every 5 weeks, as well as any time ranges in between, most preferably once every 3 weeks to a subject in need thereof.

In particular, in one embodiment of the dosing regimen of the present invention the antibody is administered at a fixed dose of 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, or 10,000 mg, as well as any ranges in between, preferably of 2500 mg, to a subject in need thereof once every week, once every 2 weeks, once every 3 weeks, once every 4 weeks, once every 5 weeks, as well as any time ranges in between, preferably once every 3 weeks. Thus, the dosing regimen of the present invention comprises administration of a dose of the antibody of 1000 mg every week, of 1000 mg every 2 weeks, of 1000 mg every 3 weeks, of 1000 mg every 4 weeks, of 1000 mg every 5 weeks, of 1500 mg every week, of 1500 mg every 2 weeks, of 1500 mg every 3 weeks, of 1500 mg every 4 weeks, of 1500 mg every 5 weeks, of 2000 mg every week, of 2000 mg every 2 weeks, of 2000 mg every 3 weeks, of 2000 mg every 4 weeks, of 2000 mg every 5 weeks, of 2500 mg every week, of 2500 mg every 2 weeks, of 2500 mg every 3 weeks, of 2500 mg every 4 weeks, of 2500 mg every 5 weeks, of 3000 mg every week, of 3000 mg every 2 weeks, of 3000 mg every 3 weeks, of 3000 mg every 4 weeks, of 3000 mg every 5 weeks, of 3500 mg every week, of 3500 mg every 2 weeks, of 3500 mg every 3 weeks, of 3500 mg every 4 weeks, of 3500 mg every 5 weeks, of 4000 mg every week, of 4000 mg every 2 weeks, of 4000 mg every 3 weeks, of 4000 mg every 4 weeks, of 4000 mg every 5 weeks, of 4500 mg every week, of 4500 mg every 2 weeks, of 4500 mg every 3 weeks, of 4500 mg every 4 weeks, of 4500 mg every 5 weeks, of 5000 mg every week, of 5000 mg every 2 weeks, of 5000 mg every 3 weeks, of 5000 mg every 4 weeks, of 5000 mg every 5 weeks, of 5500 mg every week, of 5500 mg every 2 weeks, of 5500 mg every 3 weeks, of 5500 mg every 4 weeks, of 5500 mg every 5 weeks, of 6000 mg every week, of 6000 mg every 2 weeks, of 6000 mg every 3 weeks, of 6000 mg every 4 weeks, of 6000 mg every 5 weeks, of 6500 mg every week, of 6500 mg every 2 weeks, of 6500 mg every 3 weeks, of 6500 mg every 4 weeks, of 6500 mg every 5 weeks, of 7000 mg every week, of 7000 mg every 2 weeks, of 7000 mg every 3 weeks, of 7000 mg every 4 weeks, of 7000 mg every 5 weeks, of 7500 mg every week, of 7500 mg every 2 weeks, of 7500 mg every 3 weeks, of 7500 mg every 4 weeks, of 7500 mg every 5 weeks, of 8000 mg every week, of 8000 mg every 2 weeks, of 8000 mg every 3 weeks, of 8000 mg every 4 weeks, of 8000 mg every 5 weeks, of 8500 mg every week, of 8500 mg every 2 weeks, of 8500 mg every 3 weeks, of 8500 mg every 4 weeks, of 8500 mg every 5 weeks, of 9000 mg every week, of 9000 mg every 2 weeks, of 9000 mg every 3 weeks, of 9000 mg every 4 weeks, of 9000 mg every 5 weeks, of 9500 mg every week, of 9500 mg every 2 weeks, of 9500 mg every 3 weeks, of 9500 mg every 4 weeks, of 9500 mg every 5 weeks, of 10000 mg every week, of 10000 mg every 2 weeks, of 10000 mg every 3 weeks, of 10000 mg every 4 weeks, of 10000 mg every 5 weeks, and preferably of 2500 mg every 3 weeks.

The above-described dose regime is in principle suitable for any subject to be treated, independent on its weight. As can be seen from Example 1, the weight of the patients which have been treated with 2500 mg of the antibody ranged between 60 kg and 120.9 kg. Of course, dose adjustments might be advantageous for the therapeutic effect in case the subject to be treated weights significantly more than 121 kg or less than 60 kg.

In one embodiment of the dosing regimen of the present invention, the antibody is administered at a dose of 17 mg/kg to 167 mg/kg, preferably of 17 mg/kg to 150 mg/kg, more preferably of 17 mg/kg to 125 mg/kg, more preferably of 17 mg/kg to 83 mg/kg, more preferably of 33 mg/kg to 50 mg/kg, and in particular of 17 mg/kg, 25 mg/kg, 25 mg/kg, 42 mg/kg, 50 mg/kg, 58 mg/kg, 67 mg/kg, 75 mg/kg, 83 mg/kg, 92 mg/kg, 100 mg/kg, 108 mg/kg, 117 mg/kg, 125 mg/kg, 133 mg/kg, 142 mg/kg, 150 mg/kg, 158 mg/kg, or 167 mg/kg, as well as any ranges in between, preferably of 42 mg/kg, to a subject in need thereof once every week, once every 2 weeks, once every 3 weeks, once every 4 weeks, once every 5 weeks, as well as any time ranges in between, preferably once every 3 weeks. Thus, the dosing regimen of the present invention comprises administration of a dose of the antibody of 17 mg/kg every week, of 17 mg/kg every 2 weeks, of 17 mg/kg every 3 weeks, of 17 mg/kg every 4 weeks, of 17 mg/kg every 5 weeks, of 25 mg/kg every week, of 25 mg/kg every 2 weeks, of 25 mg/kg every 3 weeks, of 25 mg/kg every 4 weeks, of 25 mg/kg every 5 weeks, of 25 mg/kg every week, of 25 mg/kg every 2 weeks, of 25 mg/kg every 3 weeks, of 25 mg/kg every 4 weeks, of 25 mg/kg every 5 weeks, of 42 mg/kg every week, of 42 mg/kg every 2 weeks, of 42 mg/kg every 3 weeks, of 42 mg/kg every 4 weeks, of 42 mg/kg every 5 weeks, of 50 mg/kg every week, of 50 mg/kg every 2 weeks, of 50 mg/kg every 3 weeks, of 50 mg/kg every 4 weeks, of 50 mg/kg every 5 weeks, of 58 mg/kg every week, of 58 mg/kg every 2 weeks, of 58 mg/kg every 3 weeks, of 58 mg/kg every 4 weeks, of 58 mg/kg every 5 weeks, of 67 mg/kg every week, of 67 mg/kg every 2 weeks, of 67 mg/kg every 3 weeks, of 67 mg/kg every 4 weeks, of 67 mg/kg every 5 weeks, of 75 mg/kg every week, of 75 mg/kg every 2 weeks, of 75 mg/kg every 3 weeks, of 75 mg/kg every 4 weeks, of 75 mg/kg every 5 weeks, of 83 mg/kg every week, of 83 mg/kg every 2 weeks, of 83 mg/kg every 3 weeks, of 83 mg/kg every 4 weeks, of 83 mg/kg every 5 weeks, of 92 mg/kg every week, of 92 mg/kg every 2 weeks, of 92 mg/kg every 3 weeks, of 92 mg/kg every 4 weeks, of 92 mg/kg every 5 weeks, of 100 mg/kg every week, of 100 mg/kg every 2 weeks, of 100 mg/kg every 3 weeks, of 100 mg/kg every 4 weeks, of 100 mg/kg every 5 weeks, of 108 mg/kg every week, of 108 mg/kg every 2 weeks, of 108 mg/kg every 3 weeks, of 108 mg/kg every 4 weeks, of 108 mg/kg every 5 weeks, of 117 mg/kg every week, of 117 mg/kg every 2 weeks, of 117 mg/kg every 3 weeks, of 117 mg/kg every 4 weeks, of 117 mg/kg every 5 weeks, of 125 mg/kg every week, of 125 mg/kg every 2 weeks, of 125 mg/kg every 3 weeks, of 125 mg/kg every 4 weeks, of 125 mg/kg every 5 weeks, of 133 mg/kg every week, of 133 mg/kg every 2 weeks, of 133 mg/kg every 3 weeks, of 133 mg/kg every 4 weeks, of 133 mg/kg every 5 weeks, of 142 mg/kg every week, of 142 mg/kg every 2 weeks, of 142 mg/kg every 3 weeks, of 142 mg/kg every 4 weeks, of 142 mg/kg every 5 weeks, of 150 mg/kg every week, of 150 mg/kg every 2 weeks, of 150 mg/kg every 3 weeks, of 150 mg/kg every 4 weeks, of 150 mg/kg every 5 weeks, of 158 mg/kg every week, of 158 mg/kg every 2 weeks, of 158 mg/kg every 3 weeks, of 158 mg/kg every 4 weeks, of 158 mg/kg every 5 weeks, of 167 mg/kg every week, of 167 mg/kg every 2 weeks, of 167 mg/kg every 3 weeks, of 167 mg/kg every 4 weeks, of 167 mg/kg every 5 weeks, and preferably of 42 mg/kg every 3 weeks.

In one embodiment of the dosing regimen of the present invention, the subject to be treated receives a loading dose of the antibody at Day 1, followed by a different maintenance dose on Day 2 and every 1 to 5 weeks, preferably 2 to 4 weeks, most preferably 3 weeks thereafter. In a preferred embodiment, the subject to be treated receives a loading dose of the antibody at Day 1, followed by a different maintenance dose on Day 2, on Day 22±3 days, on Day 43±3 days, on Day 64±3 days, on Day 85±3 days, on Day 106±3 days, on Day 127±3 days, on Day 148±3 days, and on Day 169±3 days. The maintenance dose is preferably any one of the doses mentioned above, but preferably 2500 mg and 42 mg/kg, respectively, most preferably 2500 mg. The loading dose is preferably 200 to 800 mg and 3 mg/kg to 13 mg/kg, respectively, more preferably 300 to 700 mg and 5 mg/kg to 11 mg/kg, respectively, more preferably 400 to 600 mg and 7 mg/kg and 10 mg/kg, respectively, and most preferably 500 mg and 8 mg/kg, respectively, and in particular 500 mg.

In one embodiment of the dosing regimen, treatment is maintained for at least 4 months, preferably for at least 5 months, more preferably for at least 6 months (maintenance phase). However, in general, there is no limitation of the duration of treatment and in one embodiment of the dosing regimen of the present invention treatment is performed for one, two, three or several years. In one embodiment, treatment is continued for the lifetime of the patient, until cure of the disease, as long as clinical benefit is observed, until unmanageable toxicity and/or disease progression occurs.

The term “loading dose” refers to the initial dose administered to the patient. The term “maintenance phase” refers to the second phase of a dosing regimen, which begins in a preferred embodiment at Day 2. The term “maintenance dose” (or effective dose) refers to a dose administered to the patient after the loading dose and is administered in the maintenance phase.

In one embodiment of the dosing regimen of the present invention, the antibody is administered at a concentration of about 10 mg/mL to 50 mg/mL, preferably of about 10 mg/mL to 40 mg/mL, preferably of about 20 mg/mL to 30 mg/mL, most preferably of about 20 mg/mL.

Administration of the antibody may be effected by different ways, e.g., by intravenous, intraperitoneal, subcutaneous, intramuscular, intranasal, oral, topical or intradermal administration or spinal or brain delivery. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier. In a preferred embodiment of the present invention, the antibody is administered intravenously.

In this context, as mentioned above, a drug formulation in an L-histidine/L-histidine monohydrochloride buffer at a pH of about 6.0 has been developed that is particularly suitable for intravenous administration of the anti-SOD1 antibody at a concentration of 20 mg/mL, which is described in a further aspect of the present invention below and in the Examples. Therefore, in a most preferred embodiment of the dosing regimen of the present invention, the antibody is administered in a drug formulation according to the present invention by way of any embodiment thereof disclosed herein.

In one embodiment of the dosing regimen of the present invention, the 500 mg (loading dose) and 2500 mg (maintenance or effective dose), respectively, of the antibody is administered over a period of min. 60 and max. 120 minutes, preferably over a period of 60 minutes. In one embodiment, the antibody is administered over at least 60 minutes, wherein infusion duration may be increased, or the infusion may be stopped as deemed necessary or if an infusion reaction is observed. However, intravenous administration needs to be completed preferably within not more than 120 minutes from start of infusion. When higher or lower doses of the antibody are administered, the infusion time can be appropriately increased or decreased.

Hypersensitivity reaction may occur after administration of any monoclonal antibody. Acute hypersensitivity reactions, including infusion reactions or anaphylaxis, may occur during or within hours of the infusion. Subjects should be monitored for hypotension, fever, chills, bronchospasm, angioedema, and other symptoms or signs of anaphylaxis. Infusion should be slowed or stopped, and reactions managed. In particular, in the event that significant an infusion reaction occurs, the infusion rate should be lowered, preferably by 50%. In one embodiment, if the infusion rate has been lowered, the infusion is completed at the lower rate. In one embodiment, if the infusion rate has been lowered, the infusion rate is increased again to its previous rate (before lowering), for example when the infusion reaction moderates. Accordingly, in one embodiment, the administration of the antibody with the above-described infusion rate is followed or interrupted with an about 50% lower infusion rate, preferably wherein the desired amount of the antibody, e.g., 500 mg (loading dose) and 2500 mg (maintenance or effective dose), respectively, is administered over a period of min. 120 minutes and maximum 240 minutes.

In general, no premedication should be performed before administering the antibody in accordance with the present invention. However, if an infusion reaction occurs, appropriate medication may be used as determined by the study investigator(s). If infusion reactions are observed, acetaminophen, 500 to 1000 mg and/or an antihistamine may be administered orally or intravenous 30 to 60 minutes prior to the start of infusion for subsequent dose administrations. Furthermore, acetaminophen may be administered for the treatment of headaches.

Accordingly, if headaches or infusion reactions are observed during the treatment in accordance with the present invention, but in particular if infusion reactions are observed, acetaminophen, preferably 500 to 1000 mg may be administered and preferably 30 to 60 minutes prior to the start of the administration of the antibody, in particular prior to the start of infusion of the antibody, i.e., for subsequent dose administrations. Administration of acetaminophen can be performed orally or intravenously. Instead of acetaminophen, other analgesic antipyretic agents may be administered, like acidic (nonsteroidal anti-inflammatory drugs, NSAIDs) and nonacidic (paracetamol, pyrazolinones) drugs, as well as selective cyclooxygenase-2 (COX-2) inhibitors as reviewed for example in Hinz and Brune, Handb. Exp. Pharmacol. 177 (2007), 65-93.

In addition, or alternatively, if infusion reactions are observed during the treatment in accordance with the present invention, an antihistamine may be administered preferably 30 to 60 minutes prior to the start of the infusion of the antibody, i.e., for subsequent dose administrations, wherein administration of the antihistamine can be performed orally or intravenously.

In one embodiment of the treatment in accordance with the present invention, the antihistamine and acetaminophen in the above-mentioned doses can both be administered via the above-mentioned administration routes, if infusion reactions are observed.

In one embodiment, the antibody is administered with one or more additional medicaments or therapeutic agents useful in the treatment of ALS during the treatment in accordance with the present invention. The additional agent can be, for example, a therapeutic agent art-recognized as being useful to treat ALS. The combination can also include more than one additional agent, e.g., two or three additional agents. In one embodiment, patients treated in accordance with the present invention are treated with riluzole (Rilutek®) or edavarone (Radicava®) both which are approved by the U.S. Food and Drug Administration for the treatment of ALS, or with both riluzole and edaravone. Preferably, patients are at a stable dose of riluzole and/or edaravone before treatment in accordance with the present invention starts. In one embodiment, treatment with riluzole and/or edaravone starts simultaneously with the treatment with the antibody in accordance with the present invention.

In one embodiment, the additional therapeutic agent is an agent lowering the level of SOD1, for example pyrimethamine (Lange et al. Ann. Neurol. 81 (2017), 837-848), a therapeutic agent used for gene silencing, for example morpholino oligonucleotides (MOs) or a therapeutic agent for unspecific treatment like rapamycin. Furthermore, the additional therapeutic agent can be an agent aiming at some of the specific symptoms of ALS, e.g., pain relievers or muscle relaxants. Thus, the additional therapeutic agent is preferably baclofen (Gablofen®, Kemstro®, Lioresal®) or diazepam (Diastat®, Valium®) which can help to ease cramps. Pooling of saliva in the mouth due to difficulty in swallowing is also a symptom of ALS and can be treated with different medicines being an additional therapeutic agent in accordance with the present invention. Preferably, the therapeutic agent is Elavil® (amitriptyline), trihexyphenidyl, Scopoderm® (scopolamine patch), or Robinul® (glycopyrrolate).

In one embodiment, the additional therapeutic agent is another antibody useful for the treatment of ALS, for example a C5-antibody like ravulizumab or eculizumab. Ravulizumab (also known as BNJ441, ALXN1210, or Ultomiris®) is described WO 2015/134894 A1. Eculizumab (also known as Soliris®) is described in WO 2007/106585 A1.

As described above, two forms of ALS are known, i.e., familial ALS (fALS) and sporadic ALS (sALS) and there is strong evidence that both genetic and non-genetic drivers of SOD1 misfolding result in formation of toxic conformers that initiate and drive ALS pathogenesis, i.e., fALS and sALS are driven by pathogenic SOD1 accumulation. Importantly and supporting this hypothesis, recent evidence suggests that misfolded SOD1 aggregates can also be detected in the spinal cord of sALS patients, suggesting common elements in the pathology of disease between familial and sporadic ALS (Li and Cashman, Prion 8 (2014), 33-41; Bosco et al., Nat. Neurosci. 13 (2010), 1396-403; Forsberg et al. Acta Neuropathol. 121 (2011), 623-34; Forsberg et al., PLos One 5 (2010), e11552).

Accordingly, the subject to be treated in accordance with the present invention may suffer from fALS or from sALS.

Diagnosing of fALS and sALS can be performed with the highly sensitive immunoassay as disclosed in WO 2021/185961 A1, which comprises an anti-SOD1 antibody as capture antibody for determining the presence and level, respectively, of misfolded SOD1 (mSOD1) in a body fluid from a subject. The antibody is preferably the antibody as defined further below. This assay is in particular capable of identifying with a high degree of certainty patients with sALS. Thus, in one embodiment of the present invention, the subject to be treated has been diagnosed with the immunoassay as disclosed in WO 2021/185961 A1, which content is herein incorporated by reference.

In one embodiment, ALS is defined in the subject to be treated in accordance with the present invention with the King's ALS clinical staging system (Roche et al., Brain 135 (Part 3) (2012), 847-8S2). The King's ALS system categorizes the extent to which ALS has progressed in a patient or subject based upon the occurrence of discrete milestones, defined as the first occurrence of ALS symptoms (e.g., functional involvement by weakness, wasting, spasticity, dysarthria or dysphagia of one central nervous system region defined as bulbar, upper limb, lower limb, or diaphragmatic), diagnosis, functional involvement of a second region, functional involvement of a third region, and a need for gastrostomy and non-invasive ventilation. Based upon these observable and measurable events, the King's system classifies ALS progress according to four stages: Stage 1—symptom onset with involvement of a first region; Stage 2A—diagnosis; Stage 2B—involvement of second region; Stage 3—involvement of third region; Stage 4A—need for gastrostomy; and Stage 4B—need for non-invasive ventilation.

In one embodiment, ALS is defined in the subject to be treated in accordance with the present invention as meeting the possible, laboratory-supported probable, probable, or definite criteria for a diagnosis of ALS according to the revised World Federation of Neurology El Escorial criteria.

Thus, in one embodiment, the subject to be treated in accordance with the present invention has possible, clinically probable, clinically probable-laboratory supported or definite fALS or sALS in accordance with the El-Escorial criteria or has been diagnosed of ALS as defined by the Gold Coast Criteria; progressive motor impairment documented by history or repeated clinical examination, preceded by normal motor development, and presence of upper and lower motor neuron dysfunction in at least one body region or lower motor neuron dysfunction in at least two body regions and investigations excluding other conditions.

In one embodiment, the subject to be treated in accordance with the present invention is diagnosed with ALS onset, defined as the time of onset of first muscle weakness (e.g., limb weakness, dysarthria, dysphagia, and/or shortness of breath), no more than 24 to 60 month, preferably no more than 48 months, and most preferably within past 24 month prior to being evaluated for treatment with the antibody.

In one embodiment, the subject to be treated in accordance with the present invention has an upright slow vital capacity (SVC) of at least 50% at the time of evaluation for treatment with the antibody.

As it is known that further neurodegenerative diseases like Alzheimer Disease (AD) and Parkinson Disease (PD) are associated with superoxide dismutase (SOD1) accumulation, it is prudent to expect that those diseases are also treatable when applying the dosing regimen of the present invention to patients suffering from AD and PD. Thus, the present invention also relates to a recombinant antibody that selectively binds to misfolded and/or aggregated forms of SOD1 for use in the treatment of neurodegenerative diseases associated with SOD1 accumulation, like AD or PD by administration of a dose of the antibody of 1000 mg to 10,000 mg, preferably of 1000 mg to 5000 mg, preferably of 2000 mg to 3000 mg, and most preferably of 2500 mg to a patient in need thereof once every 1 to 5 weeks, preferably once every 2 to 4 weeks, most preferably once every 3 weeks.

The antibody for use in the dosing regimen and the drug product, i.e., the liquid aqueous pharmaceutical formulation of the present invention can in principle be any anti-SOD1 antibody that selectively binds to misfolded and/or aggregated forms of SOD1, preferably preferentially or exclusively over the physiological form of SOD1.

In principle, the antibody may be any format recognizing misfolded SOD1 comprising, for example chimeric antibody, single-chain antibody, Fab-fragment, bi-specific antibody, fusion antibody, labeled antibody or an analog of any one of those. Corresponding methods for producing such variants are known to the person skilled in the art and are described, e.g., in Harlow and Lane “Antibodies, A Laboratory Manual”, CSH Press, Cold Spring Harbor (1988) First edition; Second edition by Edward A. Greenfield, Dana-Farber Cancer Institute @ 2014, ISBN 978-1-936113-81-1. For example, Fab and F(ab′) 2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

In one embodiment, the antibody as used in the dosing regimen and the drug formulation of the present invention may thus be provided in a format selected from the group consisting of a single chain Fv fragment (scFv), an F(ab′) fragment, an F (ab) fragment, and an F(ab′)2 fragment, an Fd, an Fv, a single-chain antibody, and a disulfide-linked Fv (sdFv) and/or which is a chimeric murine-human or a humanized antibody. Preferably, the anti-SOD1 antibody employed comprises a human constant domain.

The antibody AP-101, which corresponds to antibody NI-204.12G7 as characterized in WO 2012/080518 A1 and in Maier et al., Sci. Transl. Med. 10. (2018) doi: 10.1126/scitranslmed.aah3924 is particularly preferred and is a fully human IgG1m3 allotype antibody with selective high affinity binding to misfolded SOD1 protein. In two mouse models of ALS, intravenous or intrathecal administration of a chimeric version of AP-101 (cha-miSOD1) attenuated loss of spinal cord motor neurons with improved motor function and prolonged overall survival (Welt et al., Sci. Transl. Med. 10 (2018), eaah3924, WO 2012/080518 A1). The therapeutic effects of AP-101 treatment may be mediated by different non-exclusive mechanisms. These include the direct neutralization of extracellular toxic SOD1 conformers (Zhao et al., Glia 58 (2010), 231-243) and the interference with prion-like spreading of SOD1 aggregates released from dying cells or living cells via exosome-dependent or -independent pathways (Basso and Bonetto, Front. Neurosci. 10 (2016), 127; Silverman et al., Cell. Mol. Life Sci 70 (2016), 377-381), trans-synaptic propagation (Ayers et al., Acta Neuropathol. 131 (2016), 103-114), and/or macropinocytotic uptake of aggregates (Grad and Cashman, Prion 8 (2014), 33-41; Bidhendi, J Clin. Invest. 126 (2016), 2249-2253; and Sundaramoorthy et al., Cell. Mol. Life Sci. 70 (2013), 4181-4195). A functional and glycosylated fragment crystallizable (Fc) region of the antibody AP-101 provides for full therapeutic efficacy suggesting Fc gamma receptor (FcγR)-mediated effects are involved in activity in vivo. Thus, recruitment of microglia and phagocytosis of toxic SOD1 aggregates could be a plausible underlying mechanism of action (Welt et al., 2018, supra).

Thus, in one embodiment, the antibody for use in accordance with the present invention, i.e., in the dosing regimen and the drug formulation of the present invention, is an anti-SOD1 antibody recognizing an epitope comprising the amino acid sequence 73-GGPKDEERHVG-83 (SEQ ID NO: 11).

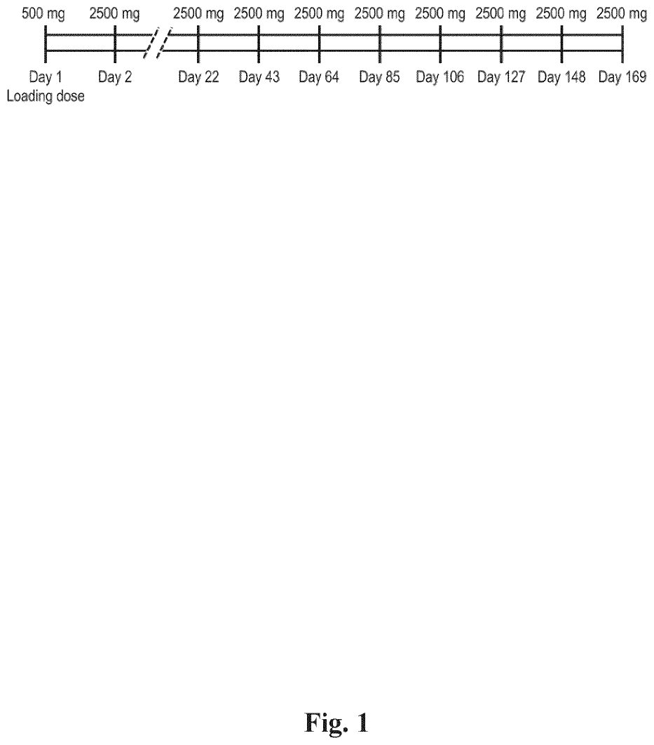

In a particular preferred embodiment the antibody for use in accordance with the present invention is AP-101 and derived from human antibody NI-204.12G7 and characterized by comprising in its variable region, i.e. binding domain the complementarity determining regions (CDRs) of the variable heavy (VH) and variable light (VL) chain having the amino acid sequences depicted in FIG. 1B of WO 2012/080518 A1, or an equivalent antibody wherein one or more of the CDRs may differ in their amino acid sequence from those set forth in FIG. 1B of WO 2012/080518 A1 by one, two, three or even more amino acids in case of CDR2 and CDR3, and wherein the antibody displays substantially the same or identical immunological characteristics of anti-SOD1 antibody NI-204.12G7 illustrated in the Examples of WO 2012/080518 A1. The positions of the CDRs are shown in FIG. 1B and explained in the Figure legend to FIG. 1 in WO 2012/080518 A1. The corresponding nucleotide sequences are set forth in Table II at page 54 of WO 2012/080518 A1. In addition, or alternatively, the framework regions or complete VH and/or VL chain are 80% identical to the framework regions depicted in FIG. 1B of WO 2012/080518 A1, preferably 85%, 90%, 95%, 96%, 97%, 98%, 99% or 100% identical to the framework regions and VH and/or VL chain, respectively, depicted in FIG. 1B of WO 2012/080518 A1. Furthermore, cloning and expression of antibody NI-204.B has been performed as described in WO 2012/080518 A1 in the section “Material and methods” at pages 84 to 88 which methods are thus incorporated herein by reference.

In a particular preferred embodiment, the antibody is characterized by the VH and/or VL chain depicted in FIG. 1B of WO 2012/080518 A1.

Thus, the antibody preferably comprises
(i) a variable heavy (VH) chain comprising VH complementary determining regions (CDRs) 1, 2, and 3, and/or a variable light (VL) chain comprising VL CDRs 1, 2, and 3, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 1 or 2 or a variant thereof, wherein the variant comprises one or more amino acid substitutions; and
   (b) the VL chain comprises the amino acid sequence depicted in SEQ ID NO: 6 or 7, or a variant thereof, wherein the variant comprises one or more amino acid substitutions;
   preferably wherein the VH and VL chain amino acid sequence is at least 90% identical to SEQ ID NO: 1 or 2 and 6 or 7, respectively.

Accordingly, most preferably the anti-SOD1 antibody to be used in the dosing regimen and drug formulation of the present invention is AP-101 or an equivalent antibody derived from human antibody NI-204.12G7 as characterized in WO 2012/080518 A1 and Maier et al., Sci. Transl. Med. 10. (2018) doi: 10.1126/scitranslmed.aah3924.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., J. Allergy Clin. Immunol. 125 (2010), S41-S52. Furthermore, different subclasses exist, wherein the IgAs are further divided into subclasses IgA1 and IgA2, and wherein IgGs are further divided into subclasses IgG1, IgG2, IgG3, and IgG4. Furthermore, two types of light chain, kappa (κ) and lambda (λ) exist.

In principle, the antibody as used in accordance with the present invention may be of any kind of class and subclass, respectively, and may comprise any kind of light chain, as long as the antibody binds to misfolded and preferably aggregated forms of SOD1, and preferably as long as binding specificity towards SOD1 as indicated in the Examples of WO 2012/080518 A1 for antibody NI-204.12G7 remains unaffected in kind and as long as no adverse effects occur when administering said antibody to a patient, wherein the adverse effects can be determined as described in Examples 1 and 2. However, preferably complete IgG antibodies are used, wherein the antibody comprises a constant domain. Accordingly, in one embodiment, the immunoglobulin heavy and/or light chain constant domain present in the antibody as used in accordance with the present invention is of the IgG type, the IgM type, the IgA type, the IgD type or the IgE type, preferably of the IgG type. In one embodiment, the immunoglobulin heavy and/or light chain constant domain present in the antibody as used in accordance with the present invention is of the IgA1, IgA1, IgG1, IgG2, IgG3, or IgG4 subclass, preferably of the IgG1, IgG2, IgG3, or IgG4 subclass and most preferably of the IgG1 subclass.

Recombinant expression of complete human IgG1 antibodies with a human or mouse constant domain can be performed substantially as described in the Examples of WO 2012/080518 A1. Preferably, the antibody is a monoclonal antibody or derived from a monoclonal antibody. As indicated in Table V in Example 10 at page 105 of WO 2012/080518 A1 describing the IgG germ line family classification by aligning the nucleotide sequences of the original antibodies with the human germ line sequences in the database Vbase (http://vbase.mrc-cpe.cam.ac.uk/) hosted by the MRC Centre for Protein Engineering (Cambridge, UK), the germ lines have been specified by their loci for heavy chains and by their Vbase entry numbers for the light chains, according to which antibody NI-204.12G7 is classified as 3a.119B4/V2-11+ and of the L-lambda type (NI-204.12G7L), i.e., comprising a lambda chain.

There are not only the above-mentioned four subclasses of IgGs but human heavy and light chain genes also exhibit extensive structural polymorphism(s) and, being closely linked, are inherited as a haplotype. Allotypic variants can be immunogenic and provoke antibody responses as a result of allo-immunization. Thus, switching the allotype can be of particular interest to provide non-immunogenic antibody therapeutics. So far, extensive allotypes (polymorphisms) are known, but focus is put on the serologically defined allotypes. Allotypes of IgG proteins are defined by the expression of unique epitope(s) recognized by unique serologic reagent(s). Allotypes expressed on the constant region of IgG heavy chain are designated as Gm (Genetic marker) together with the subclass, e.g., G1m, and the allotype number (or letter), e.g., G1m1 [or G1m(a)], G3m5 [or G3m(b1)]. Human immunoglobulin allotypes are listed in Table 1 of Jefferis and Lefrance, mAbs 1 (2009), 1-7 and in FIG. 1A of Irani et al., Molecular Immunology 67 (2015), 171-182, which content is herein incorporated by reference. Accordingly, in one embodiment, the antibody as used in accordance with the present invention is of any one of the following allotypes, but not limited thereto: G1m1, G1m2, G1m3, G1m17, G2m23, G3m21, G3m28, G3m11, G3m5, G3m13, G3m14, G3m10, G3m15, G3m16, G3m6, G3m24, G3m26, G3m27, A2 m1, A2m2, A2m3, Em1, Km1, Km2, and Km3, but preferably of G1m2, G1m3, or G1m17, and most preferably of G1m3.

As explained above, antibody AP-101 is a fully human IgG1m3 allotype antibody and composed of two identical heavy chains of the IgG1 subclass and the IgG1m3 allotype. In addition, as mentioned above, original human antibody NI-204.12G7 is of the L-lambda type and thus, AP-101 is composed of two identical light chains of the lambda subclass. The sequences of the variable heavy (VH) and variable light (VL) chains of AP-101 are set forth in SEQ ID NOs: 1 and 2 and 6 and 7, respectively, and the sequences of the corresponding human constant regions are known in the art. For example, each isotype and like the IgG1m3 isotype has a unique amino acid sequence of the constant regions of their heavy chains; see Jefferis and Lefrance (2009), supra. Thus, in one embodiment, the antibody as used in accordance with the present invention is characterized by two heavy chains, wherein each heavy chain comprises an amino acid sequence set forth in SEQ ID NO: 12, and by two light chains, wherein each light chain comprises an amino acid sequence set forth in SEQ ID NO: 13. Each heavy chain is comprised of 453 amino-acid residues, and each light chain consists of 213 amino acid residues. The four chains are stabilized by twelve intra-chain disulfide bonds and four inter-chain disulfide bonds. Each heavy chain contains a single N-linked glycosylation site at Asn303. The N-linked glycosylation structure is predominantly a fucosylated, complex biantennary glycan with 0 galactose residues (G0F) or with 1 galactose residue (G1F).

In addition, but to a minor extent and preferably in negligible amounts, some antibody species may be found in the formulation of the present invention, and as used in the dosing regimen described herein that may have undergone other post-translational modifications (PTMs) such as partial cleavage, oxidation, deamidation, succinimide or pyroglutamate formation and isomerization. The PTMs identified to be present in AP-101 are shown in Example 7 and Table 18.

In one preferred embodiment, the antibody for use in accordance with the present invention has a heavy chain that does not comprise a C-terminal lysine. For example, in such embodiment, the C-terminal lysine included in SEQ ID NO: 12 is absent.

In addition, or alternatively, the antibody for use in accordance with the present invention has a heavy chain, in which the glutamine at the N-terminal is substituted with pyroglutamate. This pyroglutamate formation is also referred to as N-terminal cyclization.

In addition, or alternatively, the antibody for use in accordance with the present invention has a heavy chain which is N-glycosylated, preferably wherein the N-linked glycosylation site is Asn303.

Most preferably, the antibody for use in accordance with the present invention has a heavy chain that does not comprise a C-terminal lysine, i.e., which C-terminal lysine has undergone C-terminal lysine clipping, in which the glutamine at the N-terminal is substituted with pyroglutamate, i.e., which has undergone N-terminal glutaminyl cyclization, and which is N-glycosylated.

The amino acid sequences of the heavy and light chains are shown below:

| 1 | QVQLVQSGAE VKKPGASVTL SCKASGYTFT AYYIHWVRQA REQGLEWMGV |
|---|---|
| 51 | INPSTGTTFY AQNFPDRVSV TRDTSTSTVF MELHNLKSED TAVYYCARAI |
| 101 | SEHGSGSYSP YYWGQGTLVT VSSASTKGPS VFPLAPSSKS TSGGTAALGC |
| 151 | LVKDYFPEPV TVSWNSGALT SGVHTFPAVL QSSGLYSLSS VVTVPSSSLG |
| 201 | TQTYICNVNH KPSNTKVDKR VEPKSCDKTH TCPPCPAPEL LGGPSVFLFP |
| 251 | PKPKDTLMIS RTPEVTCVVV DVSHEDPEVK FNWYVDGVEV HNAKTKPREE |
| 301 | QYNSTYRVVS VLTVLHQDWL NGKEYKCKVS NKALPAPIEK TISKAKGQPR |
| 351 | EPQVYTLPPS REEMTKNQVS LTCLVKGFYP SDIAVEWESN GQPENNYKTT |
| 401 | PPVLDSDGSF FLYSKLTVDK SRWQQGNVFS CSVMHEALHN HYTQKSLSLS |
| 451 | PGK |
| (SEQ ID NO: 12, AP-101, heavy chain amino acid sequence, wherein | |
| the amino acids of the constant region are underlined, and wherein | |
| the C-terminal lysine (K) is optional and/or the N-terminal | |
| glutamine (Q) undergoes intramolecular cyclization, resulting in | |
| the formation of pyroglutamic acid) | |
| 1 | SYELTQPPSV SVSLGQMAAI TCSGEALPKK YGYWYQQKPG QVPVLLIYRD |
| 51 | VERPSGVPDR FSGSSSGTMV TLTISGVQAE DEADYYCLSA DSSGTWVFGG |
| 101 | GTKLTVLGQP KAAPSVTLFP PSSEELQANK ATLVCLISDF YPGAVTVAWK |
| 151 | ADSSPVKAGV ETTTPSKQSN NKYAASSYLS LTPEQWKSHR SYSCQVTHEG |
| 201 | STVEKTVAPT ECS |

(SEQ ID NO: 13, AP-101, light chain amino acid sequence, wherein
the amino acids of the constant region are underlined)

In one embodiment, treatment of ALS in accordance with the present invention includes the amelioration or improvement of one or more symptoms associated with ALS. Symptoms associated with ALS include a progressive loss of motor neurons leading to variable amounts of weakness and spasticity in the limb, bulbar, and respiratory muscles.

In one embodiment, treatment of ALS includes an improvement of at least one clinical marker and biomarker, respectively for ALS progression. The revised ALSFRS-R is the accepted clinical endpoint for functional impact on disease progression in ALS (FDA 2019 (Guidance for Industry: Amyotrophic lateral sclerosis: developing drugs for treatment. September 2019); EMA 2015 (European Medicines Agency. Committee for medicinal products for human use (CHMP). Guideline on clinical investigation of medicinal products for the treatment of amyotrophic lateral sclerosis (ALS)). Recent data further suggests that several neurofilament proteins may have clinical utility in ALS prognosis and response to therapy. Preclinical models originally suggested that phospho-neurofilament heavy chain (pNfH) and neurofilament light chain (NfL) could serve as biomarkers for monitoring axonal degeneration during ALS (Brettschneider et al., Neurology 66 (2006), 852-856; Boylan et al., J. Neurochem. 111 (2009), 1182-1191; Gaiottino et al., PLOS One 8 (2013), e75091). These studies have been supported more recently in publications describing increases in neurofilament markers with disease onset and correlations between levels of these proteins and the overall rate of progression in ALS (Benatar et al., Neurology 95 (2020), e59-e69). Further, there is now emerging evidence that neurofilament levels in serum and CSF have potential as early markers of therapeutic efficacy (Miller et al., N. Engl. J. Med. 383 (2020), 109-119. However, further markers can be suitable and non-limiting examples of those markers for ALS progression include:
change from baseline in ALSFRS-R total score, i.e., measurement of the clinical effect of the treatment on the rate of functional decline as measured by the ALSFRS-R;
change from baseline in percent predicted SVC, i.e., measurement of the clinical effect of the treatment on lung function (SVC);
measurement of the clinical effect of the treatment on pulse oximetry;
percent change in combined muscle megascore from baseline as assessed by handheld dynamometry (HHD), i.e., measurement of the clinical effect of the treatment on muscle strength (HHD);
time to the earliest occurrence of one of the following Ventilation Assistance-Free Survival (VAFS) events during the Randomized Controlled Period: all-cause mortality; first use of non-invasive ventilation (NIV) for ˜22 hours per day for ˜10 consecutive days; and first use of permanent assisted ventilation (PAV) for ˜22 hours per day for ˜7 consecutive days, i.e., composite time to event measurement comprising time to death, tracheostomy for ventilation purposes, or non-invasive ventilation use for 22 to 24 hours per day;
change from baseline in ALSAQ-40 score;
change from baseline in European Quality of Life Health 5-item questionnaire (EQ 5D 5L);
change from baseline in Short Form Health Survey (SF 36);
change from baseline in total Treatment Satisfaction Questionnaire for Medication (TSQM) score;
shifts from baseline in Columbia-suicide severity rating scale (C-SSRS);
change from baseline in vital signs, electrocardiogram (ECG) parameters, and clinical laboratory assessments;
change from baseline in levels of biomarkers of complement dysregulation, neuroinflammation, and neurodegeneration;
any decline from baseline stage on the King's staging system, i.e., measurement of the clinical effect of the treatment on disease staging (King's staging tool);
measurement of the clinical effect of the treatment on the quality of life by Amyotrophic Lateral Sclerosis Specific Quality of Life Instrument-Short Form (ALSSQOL-SF);
change from baseline in NfL concentrations in serum, CSF and/or plasma, i.e., measurement of NfL levels in serum, CSF and/or plasma;
change from baseline in pNfH concentrations in any body fluid, preferably in serum, CSF and/or plasma, i.e., measurement of pNfH levels in any body fluid, preferably in serum, CSF and/or plasma;
change from baseline in total SOD1 concentrations in any body fluid, preferably in the CSF, i.e., measurement of total SOD1 levels in any body fluid, preferably in the CSF;
change from baseline in mSOD1 concentrations in in any body fluid, preferably the CSF, i.e., measurement of mSOD1 levels in any body fluid, preferably in the CSF; and/or
measurement of other biological markers of disease activity including creatinine and transcriptional markers.

Preferably, assessment of the markers is performed at several time points throughout the study, but at least at the endpoint, i.e., here week 24 (6 month) after beginning of the treatment. Of course, longer or shorter periods may be observed, for example until week 50.

Thus, the therapeutic effect of the antibody as used in accordance with the present invention and the dosing regimen of the present invention, respectively, and progression of ALS is monitored by assessing one or more markers, preferably selected from the markers listed above.

In a preferred embodiment of the treatment in accordance with the present invention, the therapeutic effect of the antibody and progression of ALS is monitored by assessing one or more markers, which are selected from the group consisting of:
measurement of the clinical effect of the treatment on the rate of functional decline as measured by the ALSFRS-R;
measurement of the clinical effect of the treatment on lung function (SVC);
measurement of the clinical effect of the treatment on pulse oximetry;
measurement of the clinical effect of the treatment on muscle strength (HHD);
composite time to event measurement comprising time to death, tracheostomy for ventilation purposes, or non-invasive ventilation use for 22 to 24 hours per day;
measurement of the clinical effect of the treatment on disease staging (King's staging tool);
measurement of the clinical effect of the treatment on the quality of life by ALSSQOL-SF;
measurement of NfL levels in any body fluid, preferably in serum, CSF and/or plasma, preferably in the CSF and plasma;
measurement of pNfH levels in any body fluid, preferably in serum, CSF and/or plasma, preferably in the CSF and plasma;
measurement of total SOD1 levels in any body fluid, preferably in the CSF;
measurement of mSOD1 levels in any body fluid, preferably in the CSF; and
measurement of other biological markers of disease activity including creatinine and transcriptional markers, or any one of those disclosed in Table 2 of Vijayakumar et al., Front. Neurol. 10 (2019), 400, and/or in Tables 1 to 5 disclosed in Vu and Bowser, Neurotherapeutics 14 (2017), 119-134.

In a further preferred embodiment of the treatment in accordance with the present invention, the therapeutic effect of the antibody and progression of ALS is monitored by assessing one or more markers, which are selected from the group consisting of:
measurement of NfL levels in any body fluid, preferably in serum, CSF and/or plasma, more preferably in the CSF and plasma;
measurement of pNfH levels in in any body fluid, preferably serum, CSF and/or plasma, more preferably in the CSF and plasma;
measurement of total SOD1 levels in any body fluid, preferably in the CSF; and
measurement of mSOD1 levels in any body fluid, preferably in the CSF.

In an even more preferred embodiment of the treatment in accordance with the present invention, the therapeutic effect of the antibody and progression of ALS is monitored by assessing one or more markers, which are selected from the group consisting of:
measurement of NfL levels in any body fluid, preferably in serum, CSF and/or plasma, more preferably in the CSF and plasma;
measurement of pNfH levels in in any body fluid, preferably serum, CSF and/or plasma, more preferably in the CSF and plasma; and
measurement of mSOD1 levels in any body fluid, preferably in the CSF.

As already indicated above, measurement of SOD1 levels, in particular of mSOD1 levels can be performed with the immunoassay as disclosed in WO 2021/185961 A1.

In a further aspect, but related to the dosing regimen described herein, the present invention relates to a liquid aqueous pharmaceutical formulation (drug product), of an anti-SOD1 antibody that selectively binds to misfolded and preferably aggregated forms of SOD1, wherein the formulation is characterized by a L-histidine/L-histidine monohydrochloride buffer with a pH of about 6.0±1, preferably a pH of about 6.0±0.5. As mentioned before, such formulation has been proven to be particularly suitable for intravenous administration and use in the dosing regimen of the present invention.

In a preferred embodiment, the formulation of the present invention comprises the antibody as defined hereinbefore. In one embodiment, the antibody is characterized by comprising in its variable region the six CDRs of the VH and VL chain as described hereinbefore and a constant region, preferably a human Ig constant region, and/or by comprising in its variable region the VH chain and the VL chain as described hereinbefore and a constant region, preferably human Ig constant region. The antibody comprised in the formulation of the present invention is preferably characterized by binding to an epitope of SOD1 within the amino acid sequence 73-GGPKDEERHVGD-84 set forth in SEQ ID NO: 11. In one embodiment, the antibody comprised in the formulation of the present invention is a human IgG, preferably a human IgG1, more preferably a human IgG1m3 allotype. Preferably, the antibody is defined by its heavy and light chain as described hereinbefore, wherein the light chain is preferably a lambda (λ) light chain. Most preferably, the antibody is antibody AP-101 and characterized by two heavy chains, wherein each heavy chain comprises an amino acid sequence set forth in SEQ ID NO: 12, and by two light chains, wherein each light chain comprises an amino acid sequence set forth in SEQ ID NO: 13.

In one embodiment, the antibody in the formulation of the present invention has a heavy chain that does not comprise a C-terminal lysine. For example, in such embodiment, the C-terminal lysine of SEQ ID NO: 12 is absent. Accordingly, in a preferred embodiment, the formulation of the present invention comprises an antibody as defined hereinbefore which is characterized by two heavy chains, wherein each heavy chain comprises an amino acid sequence set forth in SEQ ID NO: 12, wherein in one of the heavy chains, preferably in both of the heavy chains, the C-terminal lysine included in SEQ ID NO: 12 is absent, and by two light chains, wherein each light chain comprises an amino acid sequence set forth in SEQ ID NO: 13.

In addition, or alternatively, the antibody in the formulation of the present invention has a heavy chain, in which the glutamine at the N-terminal is substituted with pyroglutamate. This pyroglutamate formation also referred to as N-terminal cyclization. Accordingly, in a preferred embodiment, the formulation of the present invention comprises an antibody as defined hereinbefore which is characterized by two heavy chains, wherein each heavy chain comprises an amino acid sequence set forth in SEQ ID NO: 12, wherein in one of the heavy chains, preferably in both of the heavy chains, the glutamine at the N-terminal is substituted with pyroglutamate, and by two light chains, wherein each light chain comprises an amino acid sequence set forth in SEQ ID NO: 13.

In addition, or alternatively, the antibody in the formulation of the present invention has a heavy chain which is N-glycosylated, preferably wherein the N-linked glycosylation site is Asn303. Accordingly, in a preferred embodiment, the formulation of the present invention comprises an antibody as defined hereinbefore which is characterized by two heavy chains, wherein each heavy chain comprises an amino acid sequence set forth in SEQ ID NO: 12, wherein one of the heavy chains, preferably both of the heavy chains are N-glycosylated, preferably wherein the Asn at position 303 of the heavy chain is glycosylated, and by two light chains, wherein each light chain comprises an amino acid sequence set forth in SEQ ID NO: 13.

Most preferably, the antibody in the formulation of the present invention has a heavy chain that does not comprise a C-terminal lysine, i.e., which C-terminal lysine has undergone C-terminal lysine clipping, in which the glutamine at the N-terminal is substituted with pyroglutamate, i.e., which has undergone N-terminal glutaminyl cyclization, and which is N-glycosylated.

As shown in Example 7, about 99 to 100% of the antibodies in a sample of the antibody formulation of the present invention have a N-terminal pyro-glutamic acid and about 94% of the antibodies have a loss of the C-terminal lysine. Thus, in one embodiment, at least between 90% and 100%, preferably between 95% and 100% of the antibodies in the formulation have a N-terminal pyro-glutamic acid modified from N-terminal glutamine. In one embodiment, at least about 90%, preferably 91%, more preferably 92%, more preferably 93%, more preferably 94%, more preferably, 95%, more preferably 96%, more preferably 97%, more preferably 98%, more preferably 99% and most preferably 99.5% of the antibodies in the formulation have a N-terminal pyro-glutamic acid modified from N-terminal glutamine. In one embodiment, at least between 85% and 100%, preferably between 90% and 97% of the antibodies in the formulation have a loss of the C-terminal lysine. In one embodiment, at least about 85%, preferably 86%, more preferably 87%, more preferably 88%, more preferably 89%, more preferably, 90%, more preferably 91%, more preferably 92%, more preferably 93%, and most preferably 94% of the antibodies in the formulation have a loss of the C-terminal lysine.

In a preferred embodiment, the formulation of the present invention comprises the antibody in a therapeutically effective amount, preferably at a concentration of about 10 to 50 mg/mL, wherein the antibody remains stable at 5° C.±2° C. for at least 1 month, preferably for up to 24 months, more preferably for up to 53 months; or at 25° C.±2° C. for at least 1 month, preferably for up to 6 months, or at 40° C.±2° C. for 1 week; or at 5° C.±2° C. for at least 1 month, preferably for up to 24 months, more preferably for up to 53 months and at 25° C.±2° C. for at least 1 month, preferably for up to 6 months; or at 5° C.±2° C. for at least 1 month, preferably for up to 24 months, more preferably for up to 53 months and at 40° C.±2° C. for 1 week; or at 25° C.±2° C. for at least 1 month, preferably for up to 6 months and at 40° C.±2° C. for 1 week; or at 5° C.±2° C. for at least 1 month, preferably for up to 24 months, more preferably for up to 53 months and at 25° C.±2° C. for at least 1 month, preferably for up to 6 months, and at 40° C.±2° C. for 1 week.

In particular, the antibody concentration can be 10 mg/ml, 15 mg/mL, 20 mg/mL, 25 mg/mL, 30 mg/mL, 35 mg/mL, 40 mg/mL, 45 mg/mL, or 50 mg/mL or any ranges in between 10 mg/mL and 50 mg/mL. In a preferred embodiment, the formulation of the present invention comprises the antibody at a concentration of 20±5 mg/mL, preferably of 20 mg/mL. The antibody is the one as defined in accordance with the present invention and the formulation of the present invention is particularly suitable to realize the dosing regimen of the present invention.

In the context of the present invention, a “therapeutically effective amount” of the antibody refers to an amount effective in the prevention or treatment of a disorder for the treatment of which the antibody is effective, in the present case ALS or related diseases. The “pharmaceutical formulation” as referred to in the present invention is prepared in such form as to permit the active ingredient to be effective, and includes only pharmaceutically acceptable excipients, diluents, and other additives deemed safe by regulatory authorities and which contains no additional components which are toxic to the subjects to which the formulation would be administered. As used herein the phrase “liquid aqueous pharmaceutical formulation” refers to a pharmaceutical formulation as defined above in a liquid state using water as a solvent and comprising a pharmaceutically active drug in combination with one or more excipients. In this context, the term “pharmaceutical formulation” used in accordance with the present invention to define a drug formulation, alternatively referred to as “drug product”, and medicament, respectively, may not be confused with a “test formulation” only proposed as a “pharmaceutical formulation” upon hypothetical considerations, and for which tolerability to humans and/or efficacy at least in an accepted animal model has not been proven. In one embodiment, such “test formulations” are excluded from the scope of the preset invention. In a further embodiment the liquid aqueous pharmaceutical formulation of the present invention is a pharmaceutical formulation as defined above that maintains stability upon storage (e.g., chemical and/or physical stability/and/or biological activity) without the need for lyophilization, spray-drying, and/or freezing.

As used herein, the term “buffer” or “buffered solution” refer to an aqueous solution consisting of a mixture of a weak acid and its conjugate base or a weak base and its conjugate acid that resists changes in pH. It is well known that both type and concentration of a buffering agent may have an impact on the stability of proteins. Therefore, the selection of the buffering agent is crucial to achieve pH control and desired stabilization of antibody formulations. Regarding antibody aggregation, the choice of a suitable buffer system and the pH play a critical role as it determines the net charge of the protein/antibody structure and subsequent electrostatic interactions (Chi et al., Pharmaceutical Research 20 (2003), 1325-1336) relative to increasing acidity or basicity of the solution. The increasing charge repulsion between the charged groups within the antibody in solution can destabilize its folded or native state (Vermeer and Norde, Biophys. J. 76 (2000), 394-404). In the context with the present invention, it has been found that an L-histidine/L-histidine monohydrochloride buffer at a pH of about 6.0±1 is particularly suitable for the antibody formulation.

In one embodiment, the concentration of L-histidine/L-histidine monohydrochloride in the formulation of the present invention is 20±10 mM, preferably 20 mM.

In a further embodiment, any of the formulation of the present invention comprises an osmolarity of 310±50 mOsmol/kg. In a preferred embodiment, the osmolarity of the formulation of the present invention is between 290 and 320 mOsmol/kg, more preferably between 300 and 310 mOsmol/kg, and most preferably about 307 mOsmol/kg.

The formulation of the present invention allows intravenous, intraperitoneal, subcutaneous, intramuscular, intranasal, oral, topical or intradermal administration or spinal or brain delivery of the antibody. In a preferred embodiment, the formulation of the present invention is designed for intravenous administration.

In one embodiment, the formulation of the present invention comprises further excipients, for example an antioxidant, a tonicity modifier, and/or a surfactant in any combination of two or all three excipients. On the other hand, compared to the most preferred pharmaceutical formulation illustrated in the Examples, one or more excipients may be omitted from the solution or replaced by another excipient for their known properties.

As used herein the term “excipient” encloses therapeutically inactive ingredients of the pharmaceutical formulation of the present invention. Depending on their individual properties, excipients may be included in a pharmaceutical formulation for a wide variety of purposes, for example, buffering agents to control pH, carbohydrates as bulking agents for lyophilization, polymers to increase solution viscosity, salts or sugars to stabilize proteins and act as tonicity agents to obtain physiological tonicity and osmolality, surfactants to inhibit protein adsorption to interfaces, preservatives to prevent microbial growth, antioxidants, cryoprotectants or diluents. A variety of formulation excipients have been shown to stabilize antibodies under different processing conditions and during storage, including sugars such as sucrose, lactose or dextrose, polyols (also known as sugar alcohols) such as mannitol or sorbitol, salts such as sodium chloride, amino acids such as arginine, histidine, lysine, aspartic acid, or glutamic acid, surfactants and water as solvent (Paborji et al., Pharm. Res. 11 (1994), 764-771).

An “antioxidant”, or a free-radical scavenger, is a molecule capable of decreasing or preventing the oxidation of other molecule. As used herein the terms “tonicity agent” or “isotonizing agent” or “tonicity modifier” or “tonifier” or “tonicifying agent” refer to an agent which functions to render a liquid aqueous formulation similar in osmotic characteristics to physiologic fluids, in other words, the thus isotonic pharmaceutical formulation of interest has essentially the same osmotic pressure as human blood. Tonicity agents which are typically used include dextrose, mannitol, sodium chloride, potassium chloride and glycerin. Isotonic or physiologic formulations will generally have an osmotic pressure from about 275-325 mOsm. A “surfactant” as used within the present invention can be a nonionic and ionic surfactant. These surfactants drop surface tension of protein solutions and decrease the driving force for protein adsorption and or aggregation at hydrophobic surfaces. Nonionic surfactants are generally preferred in protein stabilization. Low concentrations of nonionic surfactants are often sufficient to prevent or reduce protein surface adsorption and/or aggregation due to their relatively low “critical micelle concentrations” (CMC) (Bam et al., Pharm Res. 12 (1995), 2-11). The term “surfactant” as used in the present invention therefore particularly refers to nonionic surfactants which are widely used to stabilize proteins, suppress aggregation, and assist in protein refolding (Chi et al. 2003, supra). Said surfactant is, preferably, a polysorbate, which is an emulsifier derived from PEGylated sorbitan (a derivative of sorbitol) esterified with fatty acids. Polysorbate 80 and polysorbate 20, also known as Tween 80® and Tween 20®, respectively, have been widely incorporated in marketed protein pharmaceuticals at 0.0003-0.3% range.

In one embodiment, the formulation of the present invention comprises an antioxidant. In one embodiment, the antioxidant is an amino acid or an amino acid derivative, like L-methionine, L-arginine-hydrochloride, or N-acetyl-l-cysteine, or a vitamin, like ascorbic acid, or a thiol. In a preferred embodiment, the antioxidant is an amino acid, and most preferably L-methionine. Thus, in a preferred embodiment, the formulation of the present invention comprises L-methionine, preferably at a concentration of 0.1%±0.05% (w/v), most preferably at a concentration of 0.1% (w/v).

In one embodiment, the formulation of the present invention comprises a tonicity modifier. In one embodiment, the tonicity modifier is selected from the group consisting of sucrose, trehalose, sorbitol, dextrose, mannitol, sodium chloride, potassium chloride and glycerin, or any combination thereof. In a preferred embodiment, the tonicity modifier is selected from sucrose, trehalose, sorbitol, and sodium chloride, or any combination thereof. Most preferably, the formulation of the present invention comprises sucrose, preferably at a concentration of 8%±1% (w/v), most preferably sucrose at a concentration of 8% (w/v).

In one embodiment, the formulation of the present invention comprises a surfactant, preferably a nonionic surfactant. In one embodiment, the surfactant is a poloxamer 188 or a polysorbate, preferably a polysorbate, preferably polysorbate 80 or polysorbate 20. In a preferred embodiment, the surfactant is polysorbate 80. Thus, in a preferred embodiment, the formulation of the present invention comprises polysorbate 80 at concentration of 0.02%±0.01 (w/v), most preferably polysorbate 80 at a concentration of 0.02% (w/v).

Accordingly, the formulation of the present invention comprises the antibody as defined hereinbefore in an L-histidine/L-histidine monohydrochloride buffer and may further comprise any one of the mentioned excipients. Thus, the present invention relates to a formulation which comprises an anti-SOD1 antibody as defined hereinbefore at a concentration of about 10 to 50 mg/mL, preferably of about 20±5 mg/mL, most preferably of 20 mg/mL, and an L-histidine/L-histidine monohydrochloride buffer at a pH of about 6.0±1, preferably wherein the concentration of the L-histidine/L-histidine monohydrochloride buffer is 20±10 mM, most preferably 20 mM, and preferably further comprises:
an antioxidant, preferably L-methionine, preferably L-methionine at a concentration of 0.1%±0.05% (w/v), most preferably L-methionine at a concentration of 0.1% (w/v); or
a tonicity modifier, preferably sucrose, preferably sucrose at a concentration of 8%±1% (w/v), most preferably sucrose at a concentration of 8% (w/v); or
a surfactant, preferably a polysorbate, preferably polysorbate 80, preferably polysorbate 80 at concentration of 0.02%=0.01 (w/v), most preferably polysorbate 80 at a concentration of 0.02% (w/v); or
an antioxidant, preferably L-methionine, preferably L-methionine at a concentration of 0.1%±0.05% (w/v), most preferably L-methionine at a concentration of 0.1% (w/v), and a tonicity modifier, preferably sucrose, preferably sucrose at a concentration of 8%±1% (w/v), most preferably sucrose at a concentration of 8% (w/v); or
an antioxidant, preferably L-methionine, preferably L-methionine at a concentration of 0.1%±0.05% (w/v), most preferably L-methionine at a concentration of 0.1% (w/v), and a surfactant, preferably a polysorbate, preferably polysorbate 80, preferably polysorbate 80 at concentration of 0.02%±0.01 (w/v), most preferably polysorbate 80 at a concentration of 0.02% (w/v); or
an a tonicity modifier, preferably sucrose, preferably sucrose at a concentration of 8%±1% (w/v), most preferably sucrose at a concentration of 8% (w/v), and a surfactant, preferably a polysorbate, preferably polysorbate 80, preferably polysorbate 80 at concentration of 0.02%±0.01 (w/v), most preferably polysorbate 80 at a concentration of 0.02% (w/v); or
an antioxidant, preferably L-methionine, preferably L-methionine at a concentration of 0.1%±0.05% (w/v), most preferably L-methionine at a concentration of 0.1% (w/v), and a tonicity modifier, preferably sucrose, preferably sucrose at a concentration of 8%±1% (w/v), most preferably sucrose at a concentration of 8% (w/v), and a surfactant, preferably a polysorbate, preferably polysorbate 80, preferably polysorbate 80 at concentration of 0.02%±0.01 (w/v), most preferably polysorbate 80 at a concentration of 0.02% (w/v).

In one embodiment, the formulation of the present invention may comprise one or more further excipients and in a preferred embodiment, the formulation of the present invention is substantially free of any other additional excipient(s). In a particular preferred embodiment, the formulation of the present invention is substantially free of excipient(s) other than those specifically recited hereinbefore.

The phrase “substantially free” as used throughout the disclosure of the present invention indicates the exclusion of any recited elements or group of elements and the optional inclusion of other elements, of similar or different nature than the recited elements, that do not materially change the basic or novel properties of the specified dosage regimen, method, or composition.

In one embodiment, the formulation of the present invention can have any of the above-mentioned composition provided that the antibody remains stable at 5° C.±2° C. for at least 1 month, preferably for at least 3 months, more preferably for at least 6 months, more preferably for at least 12 months, more preferably for at least 18 months, more preferably for at least 24 months, more preferably for at least 30 months, more preferably for at least 36 months, more preferably for at least 44 months, and most preferably for at least 53 months; at 25° C.±2° C. for at least 1 month, preferably for at least 3 months, most preferably for at least 6 months; and/or at 40° C.±2° C. for at least 1 week.

The stability of the formulation of the present invention can be determined by various parameters like the presence of aggregates (in particular high molecular weight species (HMWS and further low molecular weight species (LMWS))) and monomer content, respectively, the presence of acidic and basic species as well as reduction of the antibody in HC and LC as performed in Example 6.

Full IgG antibodies are expressed as tetramers consisting of two heavy (HC) and two light (LC) chains linked by disulfide bridges. During manufacturing and storage, cleavage of either HC or LC primary structure can occur due to different fragmentation mechanisms so that reduction of the antibody into LC and HC occurs, which may have negative implications for the safety and efficacy of the product. Accordingly, such reduction should be avoided.

Protein degradation, such as aggregation, must also be minimized in therapeutic antibody formulations since aggregation can lead to a lower in vivo efficacy, increased variability among batches of the therapeutic product, and perhaps most importantly, immunogenicity in patients.

Furthermore, charge variants of antibodies exist which can be analyzed by charged-based separation techniques. These variants are generally referred to as acidic or basic species as compared with the main species. Charge variants may substantially affect the in vitro and in vivo properties of antibodies and thus, those variants should also be avoided in therapeutic antibody formulations.

Thus, in one embodiment, the formulation of the present invention is regarded as stable when it has a content of aggregated species (HMWS and LMWS) of the antibody of equal to or less than 8%, preferably of equal to or less than 5%, more preferably of equal to or less than 3%, more preferably of equal to or less than 2%, and a monomer content of equal or more than 92%, preferably of equal or more than 95%, more preferably of equal to or more than 97%, more preferably of equal or more than 98%, respectively, after storage for 1 month to 3 months, preferably from 1 month to 6 months, more preferably from 1 month to 9 months, more preferably from 1 month to 12 months, more preferably from 1 month to 18 months, more preferably from 1 month to 24 months, more preferably from 1 month to 30 months, more preferably from 1 month to 36 months, more preferably from 1 month to 44 months, and most preferably from 1 month to 53 months at 5° C.±2° C. In other words, the formulation of the present invention is regarded as stable when it has a content of aggregated species (HMWS and LMWS) of the antibody of equal to or less than 8%, preferably of equal to or less than 5%, more preferably of equal to or less than 3%, more preferably of equal to or less than 2% and a monomer content of equal or more than 92%, preferably of equal or more than 95%, more preferably of equal to or more than 97%, more preferably of equal or more than 98%, respectively, after storage for at least 1 month, preferably of at least 3 months, more preferably of at least 6 months, more preferably of at least 9 months, more preferably of at least 12 months, more preferably of at least 18 months, more preferably of at least 24 months, more preferably of at least 30 months, more preferably of at least 36 months, more preferably of at least 44 months, and most preferably of at least 53 months at 5° C.±2° C. The content of aggregated species is preferably measured by SEC.

In addition, or alternatively, the formulation of the present invention is regarded as stable when it has a content of aggregated species (HMWS and LMWS) of the antibody of equal to or less than 8%, preferably of equal to or less than 5%, more preferably of equal to or less than 4% and a monomer content of equal or more than 92%, preferably of equal or more than 95%, more preferably of equal or more than 96%, respectively, after storage for 1 month to 3 months, preferably from 1 month to 6 months at 25° C.±2° C. and 60%±5% RH. In other words, the formulation of the present invention is regarded as stable when it has a content of aggregated species (HMWS and LMWS) of the antibody of equal to or less than 8%, preferably of equal to or less than 5%, more preferably of equal to or less than 4% and a monomer content of equal or more than 92%, preferably of equal or more than 95%, more preferably of equal or more than 96%, respectively, after storage for at least 1 month, preferably of at least 3 months, more preferably of at least 6 months at 25° C.±2° C. and 60%±5% RH. The content of aggregated species is preferably measured by SEC.

In addition, or alternatively, the formulation of the present invention is regarded as stable when it has a content of aggregated species (HMWS and LMWS) of the antibody of equal to or less than 8%, preferably of equal to or less than 5%, more preferably of equal to or less than 2% and a monomer content of equal or more than 92%, preferably of equal or more than 95%, more preferably of equal or more than 98%, respectively, after storage for at least 1 week at 40° C.±2° C. and 75%±5% RH. The content of aggregated species is preferably measured by SEC.

In addition, or alternatively, the formulation of the present invention is regarded as stable when it has a content of not reduced antibody (not reduced into its LC and HC) of equal or more than 90%, preferably of equal or more than 95% after storage for 1 month to 3 months, preferably from 1 month to 6 months, more preferably from 1 month to 9 months, more preferably from 1 month to 12 months, more preferably from 1 month to 18 months, more preferably from 1 month to 24 months, more preferably from 1 month to 30 months, more preferably from 1 month to 36 months, more preferably from 1 month to 44 months, and most preferably from 1 month to 53 months at 5° C.±2° C. In other words, the formulation of the present invention is regarded as stable when it has a content of not reduced antibody (not reduced into its LC and HC) of equal or more than 90%, preferably of equal or more than 95% after storage for at least 1 month, preferably of at least 3 months, more preferably of at least 6 months, more preferably of at least 9 months, more preferably of at least 12 months, more preferably of at least 18 months, more preferably of at least 24 months, more preferably of at least 30 months, more preferably of at least 36 months, more preferably of at least 44 months, and most preferably of at least 53 months at 5° C.±2° C. The content of not reduced antibody is preferably measured by reduced CS-SDS.

In addition, or alternatively, the formulation of the present invention is regarded as stable when it has a content of not reduced antibody (not reduced into its LC and HC) of equal or more than 90%, preferably of equal or more than 95% after storage for 1 month to 3 months, preferably from 1 month to 6 months. In other words, the formulation of the present invention is regarded as stable when it has a content of not reduced antibody (not reduced into its LC and HC) of equal or more than 90%, preferably of equal or more than 95% after storage for at least 1 month, preferably of at least 3 months, more preferably of at least 6 months at 25° C.±2° C. and 60%±5% RH. The content of not reduced antibody is preferably measured by reduced CS-SDS.

In addition, or alternatively, the formulation of the present invention is regarded as stable when it has a content of not reduced antibody (not reduced into its LC and HC) of equal or more than 90%, preferably of equal or more than 95% after storage for at least 1 week at 40° C.±2° C. and 75%±5% RH. The content of not reduced antibody is preferably measured by reduced CS-SDS.

In addition, or alternatively, the formulation of the present invention is regarded as stable when it has a content of acidic species of the antibody of equal to or less than 45%, preferably of equal to or less than 40%, more preferably of equal to or less than 35%, more preferably of equal or less than 30%, more preferably of equal or less than 27% after storage for 1 month to 3 months, preferably from 1 month to 6 months, more preferably from 1 month to 9 months, more preferably from 1 month to 12 months, more preferably from 1 month to 18 months, more preferably from 1 month to 24 months, more preferably from 1 month to 30 months, more preferably from 1 month to 36 months, more preferably from 1 month to 44 months, and most preferably from 1 month to 53 months at 5° C.±2° C. In other words, the formulation of the present invention is regarded as stable when it has a content of acidic species of the antibody of equal to or less than 45%, preferably of equal to or less than 40%, more preferably of equal to or less than 35%, more preferably of equal or less than 30%, more preferably of equal or less than 27% after storage for at least 1 month, preferably of at least 3 months, more preferably of at least 6 months, more preferably of at least 9 months, more preferably of at least 12 months, more preferably of at least 18 months, more preferably of at least 24 months, more preferably of at least 30 months, more preferably of at least 36 months, more preferably of at least 44 months, and most preferably of at least 53 months at 5° C.±2° C. The content of acidic species is preferably measured by cIEF.

In addition, or alternatively, the formulation of the present invention is regarded as stable when it has a content of basic species of the antibody of equal to or less than 15%, preferably of equal to or less than 10%, more preferably of equal to or less than 7% after storage for 1 month to 3 months, preferably from 1 month to 6 months, more preferably from 1 month to 9 months, more preferably from 1 month to 12 months, more preferably from 1 month to 18 months, more preferably from 1 month to 24 months, more preferably from 1 month to 30 months, more preferably from 1 month to 36 months, more preferably from 1 month to 44 months, and most preferably from 1 month to 53 months at 5° C.±2° C. In other words, the formulation of the present invention is regarded as stable when it has a content of basic species of the antibody of equal to or less than 15%, preferably of equal to or less than 10%, more preferably of equal to or less than 7% after storage for at least 1 month, preferably of at least 3 months, more preferably of at least 6 months, more preferably of at least 9 months, more preferably of at least 12 months, more preferably of at least 18 months, more preferably of at least 24 months, more preferably of at least 30 months, more preferably of at least 36 months, more preferably of at least 44 months, and most preferably of at least 53 months at 5° C.±2° C. The content of basic species is preferably measured by cIEF.

In addition, or alternatively, the formulation of the present invention is regarded as stable when it has a content of acidic species of the antibody of equal to or less than 45%, preferably of equal to or less than 40%, more preferably of equal to or less than 35%, more preferably of equal or less than 30%, more preferably of equal or less than 27% after storage for 1 month to 3 months, preferably from 1 month to 6 months. In other words, the formulation of the present invention is regarded as stable when it has a content of acidic species of the antibody of equal to or less than 45%, preferably of equal to or less than 40%, more preferably of equal to or less than 35%, more preferably of equal or less than 30%, more preferably of equal or less than 27% after storage for at least 1 month, preferably of at least 3 months, more preferably of at least 6 months at 25° C.±2° C. and 60%±5% RH. The content of acidic species is preferably measured by cIEF.

In addition, or alternatively, the formulation of the present invention is regarded as stable when it has a content of basic species of the antibody of equal to or less than 15%, preferably of equal to or less than 10%, more preferably of equal to or less than 7% after storage for 1 month to 3 months, preferably from 1 month to 6 months. In other words, the formulation of the present invention is regarded as stable when it has a content of basic species of the antibody of equal to or less than 15%, preferably of equal to or less than 10%, more preferably of equal to or less than 7% after storage for at least 1 month, preferably of at least 3 months, more preferably of at least 6 months at 25° C.±2° C. and 60%±5% RH. The content of basic species is preferably measured by cIEF.

In addition, or alternatively, the formulation of the present invention is regarded as stable when it has a content of acidic species of the antibody of equal to or less than 45%, preferably of equal to or less than 40%, more preferably of equal to or less than 35%, more preferably of equal or less than 30%, more preferably of equal or less than 27% after storage for at least 1 week at 40° C.±2° C. and 75%±5% RH. The content of acidic species is preferably measured by cIEF.

In addition, or alternatively, the formulation of the present invention is regarded as stable when it has a content of basic species of the antibody of equal to or less than 15%, preferably of equal to or less than 10%, more preferably of equal to or less than 7% after storage for at least 1 week at 40° C.±2° C. and 75%±5% RH. The content of basic species is preferably measured by cIEF.

Furthermore, the potency of the antibody should remain stable during storage as well as the appearance of the formulation, in particular clarity, color and/or visible particles, the pH, the osmolality, and the particulate matter.

Thus, in one embodiment, the antibody in the formulation of the present invention has a relative potency of 60% to 140%, more preferably of 70% to 130%, more preferably of 80% to 120%, more preferably of 90% to 110% and most preferably of 93% to 110% under all the above-mentioned storage conditions. For details, reference is made to Table 14 to Table 17. The potency is preferably determined by FRET-binding.

Most preferably, the formulation of the present invention comprises or essentially consists of 20 mg/mL anti-SOD1 antibody, preferably AP-101 in 20 mM L-histidine/L-histidine monohydrochloride buffer, 8% (w/v), sucrose, 0.1% (w/v) L-methionine and 0.02% (w/v) polysorbate 80 at pH 6.0 and preferably shows any one or a combination of any one of the above-mentioned stability criteria.

The present invention also relates to the treatment of ALS and further neurodegenerative diseases associated with SOD1 accumulation, like AD or PD, by administration of the above-described antibody according to the dosing regimen of the present invention. Preferably, the antibody is administered in form of the formulation and the medicament, respectively of the present invention. The method may further include the diagnosing of the subject to be treated as described above and/or the monitoring of the therapeutic effect of the antibody and progression of ALS as described above, for example using the highly sensitive immunoassay as disclosed in WO 2021/185961 A1; see supra.

The present invention further relates to a medicament comprising the formulation of the present invention. In one embodiment, the present invention relates to the medicament of the present invention for use in the treatment of diseases associated with SOD1 accumulation, preferably of ALS. In one embodiment, the present invention relates to the medicament of the present invention for use in accordance with the present invention.

The present invention further relates to a pharmaceutical container comprising the formulation and the medicament, respectively, of the present invention. In one embodiment, the container is a prefilled syringe, injection pen, ampoule, bottle, autoinjector, a glass vial or an infusion bag. In a preferred embodiment, the container of the present invention is an infusion bag or a class vial, more preferably a glass vial, and most preferably a single-use glass vial. In one embodiment, the container of the present invention is a single-use glass vial sealed with a rubber stopper and a flip-off cap. In one embodiment, the glass and the container, respectively is an 8 ml glass vial.

In one embodiment, the container of the present invention is the above-defined glass vial which provides about 100 mg of the antibody at a 20 mg/mL concentration. Thus, the dose strength of one vial is 100 mg. In one embodiment, the container of the present invention, in particular the vial contains an approximate 10% volume overfill.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

### Example 1: Phase-1 Study of AP-101 in ALS Patients

A multicenter, open-label, single ascending dose study (SAD) in patients with familial and sporadic ALS has been performed to assess the safety, tolerability, and pharmacokinetics of intravenous (IV) doses of AP-101 in human.

In brief, AP-101 is a fully human IgG1 antibody with high affinity and selective binding to misfolded SOD1. A 3+3 study design was employed, which allowed for expansion of each cohort to n=6 should any safety signals be observed in the first 3 participants. Following a screening period of 28 days, AP-101 was administered via IV infusion at SAD levels in 3 cohorts: 100 mg (Cohort 1), 500 mg (Cohort 2) and 2500 mg (Cohort 3). Participants were observed onsite for 24 hours following dosing and attended predetermined visits up to 12 weeks post-dose for safety assessments and collection of pharmacokinetic (PK) samples. CSF samples were collected at screening for measurements of misfolded SOD1 as an exploratory objective. Nine patients, three in each cohort, completed the study. One SAE of lower back pain was experienced at the lowest dose but was not treatment related. Overall, AP-101 was found to be safe and tolerable in single IV infusion administrations. No toxicities or reactions related to the administration of the drug up to the dose of 2500 mg were experienced.

Participants were over the age of 18, diagnosed with ALS in accordance with El Escorial criteria (Brooks et al., Amyotroph Lateral Scler Other Motor Neuron Disord. 1 (2000), 293-299), and genetic testing was performed to determine if participants carried a pathogenic SOD1 mutation. ALS symptom onset, specifically muscle weakness, was within the last 48 months prior to trial enrolment. An upright slow vital capacity (SVC)≥60% predicted for age, height, sex, and ethnic group at screening was required for inclusion. Both concomitant riluzole and edaravone were permitted, provided participants were on a stable dose for ≥30 days or had completed ≥2 cycles prior to screening, respectively. Participants using non-invasive ventilation (NIV) for >4 hours per day were excluded from the trial, as were participants who had taken other investigational products for ALS in the previous 30 days prior to enrolment or had any previous exposure to stem cell therapy.

### Definitions and Methods

### Adverse Events (AE)

A clinical trial AE was defined for the below described studies as any untoward medical event associated with the use of the study drug, whether or not it was considered related to the drug. Any injection/infusion site reactions experienced were captured as AEs and the time of day, time relative to injection/infusion, size, amount of erythema, induration, and pruritus were recorded. The severity of all AEs was graded according to the National Cancer Institute-Common Terminology Criteria for Adverse Events (NCI-CTCAE) Version 5.0. For AEs not specifically graded by these criteria, the following criteria were applied. For each episode, the highest severity grade attained was reported.
Grade 1: mild; asymptomatic or mild symptoms; clinical or diagnostic observations only; intervention not indicated.
Grade 2: moderate; minimal, local or non-invasive intervention indicated; limiting age-appropriate instrumental activities of daily living (ADL).
Grade 3: severe or medically significant but not immediately life threatening; hospitalization or prolongation of hospitalization indicated; disabling; limiting self-care ADL.
Grade 4: life-threatening consequences; urgent intervention indicated.
Grade 5: death related to AE.

Instrumental ADL refer to preparing meals, shopping for groceries or clothes, using the telephone, managing money, etc. Self-care ADL refers to bathing, dressing and undressing, feeding self, using the toilet, taking medications, and not bedridden.

### Clinical Laboratory Evaluations

If a study patient experienced elevated alanine aminotransferase (ALT) of at least and/or more than 3 times (≥3×) the upper limit of the normal range (ULN), alkaline phosphatase (ALP)≥2×ULN, or elevated total bilirubin (TBL)≥2×ULN, liver tests were repeated (within 3 to 5 days including ALT, aspartate aminotransferase (AST), ALP, TBL, direct bilirubin, gamma-glutamyl transferase (GGT)), along with creatinine kinase to confirm the abnormality, determine if it was related to hepatic toxicity, and to determine if it was increasing or decreasing. If the abnormality persisted or worsened, clinical and laboratory monitoring was initiated by the investigator based on consultation with the Sponsor. Monitoring continued until levels normalized and/or returned to approximate baseline levels. Follow-up with patients in consultation with Sponsor was required in cases of clinical indications of treatment emergent hepatic abnormality, in addition to these selected safety tests.

### Vital Sign Measurements

Vital signs (blood pressure [BP], heart rate, respiratory rate, and temperature) were measured at predetermined time points. Single measurements were taken at screening and study site admission. Supine triplicate BP and pulse rate were collected at all other time points. Body temperature was obtained as single measurement. Pre-dose vital signs were taken approximately 1 hour prior to the schedule dosing. At time points when orthostatic measurements were obtained, patients were supine for at least 5 minutes and stood for approximately 2 minutes. When triplicate BP or pulse rate measurements preceded the orthostatic measurement, the last supine BP or pulse rate measurement was used for orthostatic calculations. If the patient felt unable to stand, supine vital signs only were recorded. If a CS deterioration was noted, the changes were documented as AEs on the eCRF.

### Physical Examination

Physical examination was performed based upon the Investigator's judgment. CS findings in physical examinations were recorded as medical history if they were observed at baseline. Otherwise, they were recorded as AEs. The subject's height was measured during the initial physical examination performed during the Screening visit. Body weight was measured at predetermined time points. If CS deterioration was noted, the change was documented as an AE on the eCRF. The Investigator continued to monitor the subject until the parameter returned to its Baseline status or until agreement was reached between the Investigator and Sponsor.

### Neurological Examinations

A directed neurological examination was performed by the investigator or designee at predetermined time points. If abnormalities were noted at these time points, additional examinations were performed until the patient returned to baseline. Mandated elements of the examination included inspection for cranial nerves, tremor, extraocular movements, brachial and patellar deep tendon reflexes, finger-nose pointing, and Romberg sign.

### Electrocardiogram

Standard 12-lead ECGs were recorded. The ECGs were interpreted by a qualified physician (the investigator or qualified designee) at the site as soon after the time of ECG collection as possible. The investigator or qualified designee was to document whether the ECG was normal or abnormal; if abnormal, whether the ECG tracing was NCS or CS. CS deterioration was to be reported as an AE.

### C-SSRS

C-SSRS (mental health as assessed by suicidal tendencies) were collected at predetermined time points. The C-SSRS questionnaire assesses ideation of suicide, its potential intensity, and behaviors intended to carry out those tendencies. Each patient answered the C-SSRS at screening twice, once in reference to their entire lifetime and once in reference to the past month/year. Each patient answered the questionnaire again at EOS and any changes in responses to each question were recorded.

### AP-101 Concentration Measurements

AP-101 concentrations in human serum and CSF were determined using an immunoassay with electrochemiluminescent (ECL) detection, validated in the 100.0-3200.0 ng/ml range (serum) and 10.0-1280.0 ng/mL range (CSF) at Nexelis (Seattle, WA).

### ALSFRS-R

The responses of each patient to the questionnaires of ALSFRS-R (functional in life skills for ALS patients) were collected over the time course of the study. The ALSFRS-R is a clinician administered, validated rating instrument for monitoring the progression of disability in patients with ALS in terms of function in life skills. Its scores correlate significantly with results of testing using the Sickness Impact Profile (Cedarbaum et al., Neurol. Sci. 169 (1999), 13-21; Damiano et al., Med. Care 37 (1999), 15-26), commonly used to measure health and quality of life. The ALSFRS-R is composed of twelve categories of life skills with one question each scored from 0 to 4. A rating of zero (0) represents complete loss of function and 4 represents full function (Cedarbaum et al. 1999, supra). Overall ALSFRS-R scores were calculated as summations of responses to the 12 questions. The ALSFRS-R was answered by each patient at screening, pre-dose (Day 1), follow-up (Day 28), and EOS (Day 84). Changes from baseline in the ALSFRS-R were calculated and presented for each patient.

### SVC

Changes in respiratory function as measured by % SVC was assessed for each patient over the time course of the study. SVC, the volume of air released through a slow, unforced expiration is a standard means for assessing airflow limitation. Exhalation occurs, in part from the elastic recoil of the respiratory system, in addition to the activity of expiratory muscles. Elastic recoil is commonly lost in ALS due to poorly understood degeneration of respiratory motor nerves and associated losses in respiratory muscle control and plasticity. SVC correlates with clinically meaningful events in ALS such as use of assisted ventilation and tracheostomy and ALS patients with slower declines in SVC have been shown to survive longer. Based on these findings, SVC is used as a clinical prognostic marker to indicate disease progression in ALS (Andrews et al., JAMA Neurol 75 (2018), 58-64). Percent (%) SVC is a measure of respiratory function and is expected to decline over time with the progression of disease in ALS patients. Upright % SVC was determined by performing 3-5 measures at screening, pre-dose (Day 1), follow-up (Day 28), and EOS (Day 84). Changes from baseline to Day 28 and to EOS were calculated and listed for each patient.

### Safety Endpoint Methodology

Safety analyses were conducted for all enrolled patients in accordance with the SAP, whether or not they completed all protocol requirements. For continuous variables, summary statistics included number of patients, mean, median, standard deviation (SD), minimum, and maximum. Categorical endpoints were summarized using number of patients, frequency, and percentages. All treatment and protocol procedure AEs were recorded and listed. When the frequency of events allowed, safety data was summarized using descriptive methodology.

### Pharmacokinetic Endpoint Methodology

PK analyses were conducted on data from all patients who received the study drug and had evaluable PK. Both a non-compartmental analysis (NCA) and a 2-compartment model were utilized to elucidate the PK of AP 101. In the NCA, serum concentrations of AP-101 and actual sample collection times were used to determine individual PK parameters using standard non-compartmental procedures, with R 4.0.4 (Vienna, Austria). Main PK parameters calculated included Cₘₐₓ (maximum observed drug concentration), tₘₐₓ (time to maximum observed drug concentration), AUC₀₋ₜ, AUC_{0-inf} (area under the drug concentration-time curve), t_{1/2} (terminal elimination half-life), and λz (first-order terminal elimination rate constant). The 2-compartment model was developed using NONMEM, version 7.4 (San Francisco, CA). Parameters such as CL and volume of distribution (V_{d}) were predicted from the model, rather than from the NCA, given the 2-compartment PK profile exhibited by AP-101. Individual serum AP-101 PK parameters were calculated using NCA. Actual sampling times were used for parameter derivations.

### Trial Design and Assessments

This was a non-randomized, open-label, first-in-human, single ascending dose (SAD) trial. A “3+3” study design was employed and allowed for expansion of each cohort to n=6 should any safety signals be observed in the first three participants in that cohort. After a screening period of 28 days, participants received a single dose of AP-101 intravenously (IV) at the dose level determined by their cohort: 100 mg in Cohort 1, 500 mg in Cohort 2, or 2500 mg in Cohort 3.

In particular, AP-101 was administered via IV infusion over at least 60 minutes and up to 120 minutes. The AP-101 drug product was formulated for IV administration in accordance with Examples 3 and 4. AP-101 for injection was supplied as a solution in a single-use glass vial. The vial was manufactured to provide 100 mg of AP-101 at a 20 mg/mL concentration. In order to ensure complete withdrawal and delivery of the label amount of 100 mg of AP-101, vials contained an approximate 10% volume overfill. The drug product was filled into a Type I glass vials sealed with a rubber stopper and a flip-off cap. The study drug was administered as an IV infusion over at least 60 minutes and up to 120 minutes. The volumes of infusions were 5 ml, 25 ml, and 125 ml in order to administer doses of AP-101 of 100 mg, 500 mg, and 2500 mg.

Sentinel dosing periods of 21 days for Cohort 1, and seven days for Cohorts 2 and 3, were used, with safety data reviewed by the Safety Review Committee (SRC) prior to dosing of subsequent participants in the cohort. Participants were admitted for a minimum of 24 hours post-dose and returned to the clinical trial site for up to 12 weeks post-dose for safety assessments and collection of PK samples.

The primary endpoints were incidence of treatment-emergent adverse events (TEAEs) and serious adverse events (SAEs), as well as incidence of abnormalities in the clinical safety measures (vital signs, clinical laboratory assessments, physical exams, neurological exams, and electrocardiograms (ECGs)). As is common for investigational products with a neurological indication, suicidal ideation was evaluated using the Columbia Suicide Severity Rating Scale (C-SSRS). The secondary endpoints were measures of pharmacokinetics in both serum and cerebrospinal fluid (CSF). Exploratory endpoints included change in quality of life and function, as assessed by the ALS Functional Rating Scale Revised (ALSFRS-R), and change in respiratory function, as assessed using SVC. CSF levels of misfolded SOD1 were also included as an exploratory measure.

### Statistical Analysis

The full analysis set (FAS) consisted of all participants enrolled who received one dose of AP-101, which consisted of nine participants. All treatment and protocol procedure adverse events (AEs) and their frequency were recorded, categorized, and summarized using descriptive methodology. Summary statistics of each cohort were summarized by dose level. Standard descriptive statistics were used to summarize the safety parameters of this study. Results of individual question responses from C-SSRS assessments were listed by participant, visit, timepoint, and dose. No further analyses were conducted for the C-SSRS results.

Serum AP-101 PK parameters (e.g., Cₘₐₓ and AUC) were calculated using non-compartmental analysis (NCA) and 2-compartment modelling. Actual sampling times were used in the case of parameter derivations. PK parameters were summarized by dose level using descriptive statistics and mean and individual AP-101 serum concentration time curves were derived. CSF PK, as a secondary objective, was taken at a unique time point for each participant in Cohorts 2 and 3.

The numerically rated responses to each question of the ALSFRS-R were listed by time point, participant, and cohort. Changes in participants' total ALSFRS-R score were reported from baseline to Day 28, and to end of study (EOS). Results of individual SVC tests, measured as % SVC, were listed according to time point, participant, and cohort. Changes in % SVC from baseline to Day 28 and EOS were calculated and reported.

### Results

Nine participants were screened and enrolled into AP-101-01. All participants met El Escorial criteria for diagnosis of ALS, with eight having definite or probable sALS, and one having fALS due to a pathogenic variant in the SOD1 gene. Three participants were enrolled in each dosing cohort, and all participants completed the study.

Participant characteristics are detailed in Table 1. Due to the small sample size of this safety study, variability in baseline characteristics is observed.

TABLE 1
Baseline demographic and disease characteristics.

| | | Cohort 1 | | Cohort 2 | | Cohort 3 | | | |
|---|---|---|---|---|---|---|---|---|---|
| | | 100 mg AP- | | 500 mg AP- | | 2500 mg | | All treated | |
| | | 101 | | 101 | | AP-101 | | participants | |
| Demographics | | n = 3 | | n = 3 | | n = 3 | | n = 9 | |
| Age | Years, | 55.03 | (9.07) | 57.3 | (17.47) | 64.0 | (11.79) | 58.9 | (12.13) |
| | mean (SD) | | | | | | | | |
| Sex | Male, n (%) | 1 | (33%) | 3 | (100%) | 2 | (67%) | 6 | (67%) |
| Race | White, n | 3 | (100%) | 3 | (100%) | 3 | (100%) | 9 | (100%) |
| | (%) | | | | | | | | |
| BMI | Kg/m², | 31.600 | (0.7937) | 24.500 | (2.0518) | 28.267 | (5.3257) | 28.122 | (4.2148) |
| | mean (SD) | | | | | | | | |
| ALSFRS-R | Total score, | 31.7 | (4.16) | 37.3 | (5.03) | 38.2 | (3.21) | 35.8 | (4.79) |
| | mean (SD) | | | | | | | | |
| SVC | % | 76.7 | (30.66) | 86.7 | (5.51) | 116 | (25.46) | 88.38 | (28.91) |
| | predicted, | | | | | | | | |
| | mean (SD) | | | | | | | | |
| Time from | Months, | | | | | | | | |
| symptom | mean (SD) | | | | | | | | |
| onset | | | | | | | | | |
| Pathogenic | n (%) | 0 | (0%) | 0 | (0%) | 1 | (33%) | 1 | (11%) |
| SOD1 | | | | | | | | | |
| mutation | | | | | | | | | |
| Concomitant | n (%) | 3 | (100%) | 1 | (33%) | 2 | (67%) | 6 | (67%) |
| riluzole | | | | | | | | | |
| Concomitant | n (%) | 1 | (33%) | 1 | (33%) | 2 | (67%) | 4 | (44%) |
| edaravone | | | | | | | | | |

### Safety

Overall, AP-101 was found to be safe and tolerable in a single IV infusion administration. 47 Treatment-emergent adverse events (TEAEs) were reported, defined as an AE occurring after the administration of the study drug. 20 at the 100 mg dose level, 6 at the 500 mg dose level, and 21 at the 2500 mg dose level. Of the patients treated, 8 (89%) experienced a TEAE All patients (100%) in Cohort 1 and Cohort 3 and (67%) in Cohort 2 experienced AEs. The most reported TEAEs were Falls (7 events), Headaches (6 events), and Fatigue (3 events). Two (2) Grade 2 non-ALS TEAEs were recorded: One (1) Fatigue event and one (1) Back pain events, but none of the TEAEs were treatment related. No dose-limiting toxicities, treatment-related TEAEs, or reactions related to the administration of the investigational product were observed.

No abnormal clinical laboratory test results were recorded in the study. No clinically significant (CS) abnormalities in vital signs were found. All but 1 patient (89%) experienced at least one abnormal physical examination and all patients (100%) experienced at least one abnormal neurological examination across all time points. Six (6) of 9 patients (67%) experienced an abnormal ECG recording across all time points: One (1) patient in Cohort 1 (33% of Cohort 1, N=1), 2 patients in Cohort 2 (67%, N=2), and 3 patients in Cohort 3 (100%, N=3). All but one patient experienced an abnormal reading at pre-dose.

Most baseline physical and neurological examinations screening results were abnormal and the majority of these results were attributable to ALS. The majority of participants had no shifts from baseline values in physical and neurological examination results. Non-ALS related abnormal findings at time points beyond screening included musculoskeletal injury and neuropathy attributed to a laboratory chemistry sampling. Rash, Gingival abscess, and wheezing were also recorded. All abnormal examination results were not clinically significant (NCS). ECG abnormalities recorded as shifts from baseline were common, but none were correlated with AEs and all were deemed NCS. No correlations between the administrations of the study drug and the physical conditions or cardiac functions of the patients were evident. Changes in mental health status were measured using the C-SSRS. While one participant did indicate that they had had suicidal ideations during their lifetime, no participants indicated suicidal ideation or behavior either prior to or recent to screening, or at the end of the study.

### Pharmacokinetics (PK)

Blood samples for PK analysis were collected pre-dose, and at the following time points following IV administration of AP-101:5 minutes, and 0.25, 0.5 1, 4, 8, 12, 24, 48, and 72 hours post-dose. AP-101 median tₘₐₓ was 0.0833-7.63 hours (minimum-maximum) across all three dose levels. Of note, AP-101 concentrations demonstrated unusual behaviour towards the end of the IV infusion, where tₘₐₓ did not occur at the first timepoint post-infusion. AP-101 exposure generally increased in proportion to dose. Variability (CV %) was moderate to high for exposure (Cₘₐₓ and AUC_{0-inf}) and ranged from 25.9 to 35.1 for Cₘₐₓ and 20.2 to 37.7 for AUC_{0-inf}. In the 500 mg dose group, two patients had an AUC_{0-inf} for which the extrapolated portion was greater than 20%. One patient in the 2500 mg dose group had an extrapolated portion for AUC_{0-inf} that was greater than 20% as well, and these patients were therefore excluded from summary statistics. Geometric mean elimination half-life (t_{1/2}) values appeared to be similar for the 100 mg and 500 mg doses, and lower for the 2500 mg dose. The PK model-predicted value for t_{1/2} was approximately 244 hours.

### Efficacy

Evaluation of change from baseline in ALSFRS-R score and change from baseline in % SVC were included as exploratory efficacy endpoints. Levels of misfolded SOD1 in the CSF at baseline was performed as an exploratory analysis.

ALSFRS-R was collected at screening, pre-dose (baseline), Day 28, and at EOS. ALSFRS-R was assessed as change from baseline (pre-dose) of functional status losses or gains, at EOS, in the total summed score. Losses in overall functional status were experienced by 5 participants, with losses ranging from 1 to 8 points on the ALSFRS-R total score. Two participants showed no functional change, and two participants showing gains in functional status, with increases of 2 to 4 points on the ALSFRS-R.

SVC was to be collected at screening, pre-dose (baseline), Day 28, and at EOS; however, due to staffing constraints and COVID-19 restrictions, five SVC time points were missed for four participants. Furthermore, SVC could not be assessed at EOS for three of the participants, nor at baseline for an additional one participant. Acknowledging these variables in the data, overall % SVC declined from baseline to the last observed SVC (which was Day 28 for three participants), with the range of decline being 2% to 32%. One participant experienced no change in % SVC, while two participants experienced an increase, ranging from 3% to 5%, in % SVC from baseline to EOS.

### SUMMARY

In summary, given the data surrounding misfolded SOD1 pathology in both sALS and SOD1-fALS, AP-101 is a fully human IgG1 antibody with high affinity and selective binding to misfolded SOD1 protein. This was a first-in-human, single ascending dose, non-randomized, open-label study of AP-101, conducted at multiple centers in Canada. Overall, AP-101 was found to be safe and tolerable in single IV infusion administrations. No correlations could be made between any TEAEs, SAE, or other abnormal findings and the investigational product administration. No dose-limiting toxicities or IV infusion-related reactions were observed.

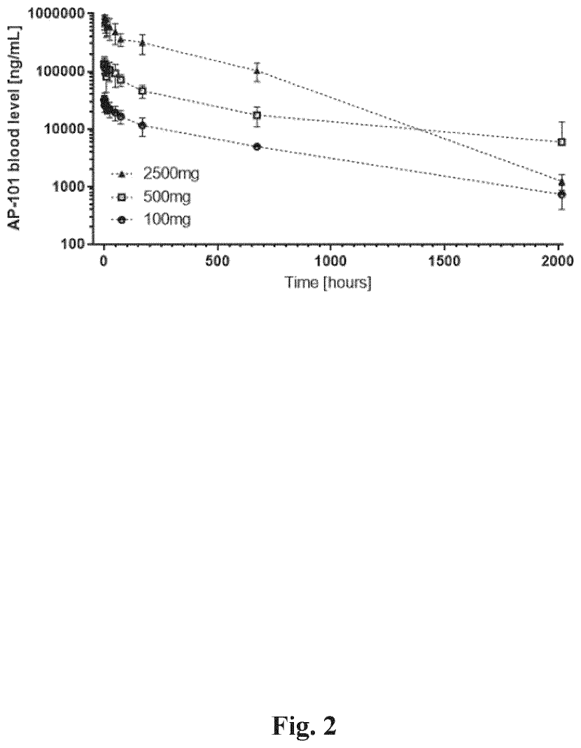

PK analyses indicate that AP-101 exposure is generally proportional to dose and clearance is biexponential (see FIG. 2), but as explained above, the PK profile is also variable. Time versus concentration profiles show that AP-101 is eliminated from the body slowly. In the lowest dose group (100 mg) the mean concentration was 731 ng/ml at Day 84 (2016 hours). The analyses and further modelling support the conclusions that AP-101 has a PK profile consistent with dosing every 3 weeks and that a 500 mg loading dose may help to rapidly achieve a desirable steady state. These analyses suggest that elimination of AP-101 is slow and that dosing every 3 weeks will provide sufficiently constant levels of the drug.

Changes from baseline in both ALSFRS-R and SVC were included as exploratory measures, and overall more loss in function was experienced than gains. Without a control group or enrolment strategy to account for heterogeneity in disease state, conclusions cannot be made regarding AP-101's effect on functional or respiratory status.

### Example 2: Phase 2a Study of AP-101 in ALS Patients

A Phase 2a, multicenter, randomized, double-blind, placebo-controlled study of AP-101 in patients with familial amyotrophic lateral sclerosis (fALS) and sporadic amyotrophic lateral sclerosis (sALS) is conducted to evaluate, safety, tolerability, pharmacodynamic (PD) markers, and pharmacokinetics (PK); see also ClinicalTrials.gov Identifier: NCT05039099. AP-101 has been administered to humans in a single ascending dose study and was found to be safe and tolerable up to doses including 2500 mg (see Example 1).

Two participant cohorts will participate in this study: fALS patients with confirmed mutations in SOD1 and patients with sALS. Patients in each participant cohort will be randomly assigned in a 2:1 ratio to either AP-101 treatment or to placebo. Patients will be allowed to continue on riluzole or edaravone per the inclusion/exclusion criteria for the duration of the study. Following completion of 6 months of treatment, participants will be invited to join a 6-months open-label extension (OLE) that will continue to evaluate long-term safety for AP-101. As well as safety and tolerability, the goal of the current study is to determine the impact of AP-101 on measurable levels of several ALS associated biomarkers. The revised ALSFRS-R is the accepted clinical endpoint for functional impact on disease progression in ALS (FDA 2019; EMA 2015). Based on these lines of evidence, the current study will look at the effects of AP-101 on multiple potential biomarkers of disease including SOD1, misfolded SOD1, pNfH and NfL. The primary, and secondary objectives of the study are indicated in the Study Details of the Phase 2a study published under ClinicalTrials.gov Identifier: NCT05039099

Potential participants with an onset of symptoms within 24 months of screening will be screened in the 28 days prior to baseline (Day 1). Study eligibility for each participant will be reviewed based on all enrollment criteria outlined below. Screening procedures are displayed in the SoE depicted in Table 2.

TABLE 2
Schedule of Events.

| | | | | | | | | | | | | | Safety/Survival | | |
|---|---|---|---|---|---|---|---|---|---|---|---|---|---|---|---|
| | | | | | | | | | | | | | Follow- Up or Early | | |
| | | | | | | | | | | | | | Termination (ET) | | |
| | | | | | | | | | | | | | 28 | 8 | 12 |
| | Visit/Week | | | | | | | | | | | | days | weeks | weeks |
| | | Treatment Period | | | | | | | | | | | post | post | post |
| | | Base- | | | | | | | | | | | last | last | last |
| | Screen- | line/ | Dose | Phone | W | W | W | W | W | W | W | W | dose | dose | dose |
| | ing | Dose 1^{a} | 2 | call^{b} | 3 | 6 | 9 | 12 | 15 | 18 | 21 | 24 | or ET | or ET | or ET |
| | Day | | | | | | | | | | | | | | |
| | | | | | 22 ± | 43 ± | 64 ± | 85 ± | 106 ± | 127 ± | 148 ± | 169 ± | | | |
| | −28 | | | | 3 | 3 | 3 | 3 | 3 | 3 | 3 | 3 | ±7 | ±7 | ±7 |
| | to −1 | 1 | 2 | 3 | days | days | days | days | days | days | days | days | days | days | days |
| Informed | X | | | | | | | | | | | | | | |
| consent | | | | | | | | | | | | | | | |
| Medical | X | | | | | | | | | | | | | | |
| history | | | | | | | | | | | | | | | |
| ALS | X | | | | | | | | | | | | | | |
| history | | | | | | | | | | | | | | | |
| Eligibility | X | X | | | | | | | | | | | | | |
| criteria | | | | | | | | | | | | | | | |
| Demo- | X | | | | | | | | | | | | | | |
| graphics | | | | | | | | | | | | | | | |
| HBsAg & | X | | | | | | | | | | | | | | |
| HCV | | | | | | | | | | | | | | | |
| Random- | | X | | | | | | | | | | | | | |
| ization | | | | | | | | | | | | | | | |
| IV dose | | X^{a} | X^{a} | | X | X | X | X | X | X | X | X | | | |
| ALSFRS-R | X | X | | | | | | X | | | | X | X | X | X |
| SVC | X | X | | | | | | X | | | | X | X | X | X |
| King's | X | X | | | | | | X | | | | X | X | X | X |
| staging | | | | | | | | | | | | | | | |
| Muscle | | X | | | | | | X | | | | X | X | X | X |
| strength | | | | | | | | | | | | | | | |
| (HHD) | | | | | | | | | | | | | | | |
| Pulse | | X | X | | X | X | X | X | X | X | X | X | X | X | X |
| oximetry^{c} | | | | | | | | | | | | | | | |
| Physical | X | X | X | | X | | | X | | | | X | X | X | X |
| exam | | | | | | | | | | | | | | | |
| Neurological | X | X | X | | X | | | X | | | | X | X | X | X |
| exam | | | | | | | | | | | | | | | |
| Concomitant | X | X | X | X | X | X | X | X | X | X | X | X | X | X | X |
| CSSR-S | X | X | | | X | X | X | X | X | X | X | X | X | X | X |
| ALSSQOL-SF | X | X | | | | | | X | | | | X | X | X | X |
| Survival | | X | | | | | | X | | | | X | X | X | X |
| endpoints^{d} | | | | | | | | | | | | | | | |
| Vital | X | X | X | | X | X | X | X | X | X | X | X | X | X | X |
| signs | | | | | | | | | | | | | | | |
| Height | X | | | | | | | | | | | | | | |
| Weight | X | X | X | | X | X | X | X | X | X | X | X | X | X | X |
| 12-Lead | X | X | X | | X | X | X | X | X | X | X | X | X | X | X |
| ECG^{e} | | | | | | | | | | | | | | | |
| Adverse | X | X | X | X | X | X | X | X | X | X | X | X | X | X | X |
| events | | | | | | | | | | | | | | | |
| Safety | X | X | X | | X | X | X | X | X | X | X | X | X | X | X |
| laboratory | | | | | | | | | | | | | | | |
| Immuno- | | X | X | | X | X | X | X | X | X | X | X | X | X | X |
| genicity | | | | | | | | | | | | | | | |
| Urine | X | X | X | | X | X | X | X | X | X | X | X | X | X | X |
| pregnancy | | | | | | | | | | | | | | | |
| test (local | | | | | | | | | | | | | | | |
| test) | | | | | | | | | | | | | | | |
| PK | | X | X | | X | X | X | X | X | X | X | X | X | X | X |
| sampling^{f} | | | | | | | | | | | | | | | |
| CSF | X | | | | | | | X | | | | X | | | X |
| PK and | | | | | | | | | | | | | | | |
| Coagulation | X | | | | | | X | | | | X | | | | |
| testing | | | | | | | | | | | | | | | |
| Serum | | X | X | | X | X | X | X | X | X | X | X | X | X | X |
| biomarkers | | | | | | | | | | | | | | | |

Abbreviations: ALS = amyotrophic lateral sclerosis; ALSFRS R = Amyotrophic Lateral Sclerosis Functional Rating Scale - Revised; ALSSQOL-SF = Amyotrophic Lateral Sclerosis Specific Quality of Life Instrument-Short Form; CSF = cerebrospinal fluid; CSSR S = Columbia Suicide Severity Rating Scale; ECG = electrocardiogram; HBsAg = surface antigen of the hepatitis B virus; HCV = hepatitis C virus; HHD = hand-held dynamometry; IV = intravenous; PK = pharmacokinetic; SVC = slow vital capacity; W = week.
Participants discontinuing from the study at any point (end of treatment period, end of 6-month OLE or early termination for any reason) should return to the site 28 days after their last dose of treatment and undergo all assessments scheduled for the 28-day post-last dose visit, and then return to the site 8 weeks and 12 weeks after their last dose of treatment and undergo all assessments scheduled per the SOE. Visit windows of ±3 days allow for flexibility in scheduling (i.e., if dosing takes place on a Friday, the next visit can be scheduled for a Monday).
Note:
If multiple procedures take place at same time point, the following order of procedures should be used: ECG, vital signs, and venipuncture. Unless otherwise specified, pre-dose sample may be taken at any time prior to dosing, based on study site activity schedule
^{a}After randomization, all participants will receive either placebo or AP-101 according to the schedule of events. On Day 1, participants will receive 500 mg of AP-101 or placebo, and on Day 2, participants will receive 2500 mg of AP-101 or placebo. Thereafter, the dose schedule will follow the SoE with dosing with 2500 mg of AP-101 or placebo every 3 weeks starting at Day 22. The observation period after IV infusion is 1 hour, or can be extended to 3 to 6 hours, as per local site procedures, if an infusion-related reaction occurs.
^{b}Phone call will take place approximately 24 hours after the baseline dose.
^{c}Pulse oximetry can be done at any time prior to dose and can be performed between other assessments.
^{d}Survival endpoints will include time to event measurements comprising time to death, tracheostomy for ventilation purposes, or non-invasive ventilation use for 22 to 24 h per day.
^{e}Single ECG will be collected at screening. Triplicate ECGs will be collected at all other time points A minimum of 1 min can be observed in between each ECGs.
^{f}A single serum PK sample will be collected pre-dose and immediately following injection during all visits except for Week 24, when PK sampling will be performed pre-dose and at 4 (+/−2) hours post-dose. At 28 D, 8 W and 12 W post final dose only a single PK sample will be taken and can be done at any time during the visit. Times of PK sampling are intended as a guide and can be modified to accommodate clinical procedures. Actual date and time of sample must be collected. For participants who discontinue early, a sample should be obtained at the ED visit.
^{g}CSF samples for PK and biomarkers: Lumbar puncture (LP) at Week 12 and Week 24 may be done 1-3 days before dosing.

Participants will receive a 500 mg AP-101 loading dose on Day 1, and then regularly scheduled doses of 2500 mg will begin on Day 2. After Day 2, dosing will take place once every 3 weeks for a 6-month period starting on Day 22 and following the SoE (Table 2). After the final dose or following early termination for any reason at any point in the study, participants will be followed for safety and PK for 3 months after their final dose of study treatment.

Safety/tolerability, PK, and exploratory assessments will be performed at prescribed time points during the study. Cerebrospinal fluid (CSF) and other biological samples for biomarker analyses will also be collected from each participant according to the SoE. Efficacy measures (amyotrophic lateral sclerosis functional rating scale-revised [ALSFRS-R], slow vital capacity [SVC], pulse oximetry, hand-held dynamometry [HHD]) and King's staging tool will be evaluated according to the SoE.

Following completion of 6 months of treatment, participants will be invited to join a 6-month open-label extension (OLE) that will continue to evaluate long-term safety for AP-101. If participating in the OLE, patients will complete the planned safety and PK follow-ups after receiving their final dose of AP-101.

A maximum of 63 participants (21 with fALS and 42 with sALS) will be randomly assigned to study intervention such that approximately 18 evaluable participants with fALS and 36 evaluable participants with sALS will complete the study. Participants who withdraw from the study before completion of all study activities may be replaced at the discretion of the sponsor. A participant completes the study when Week 24 dosing and all scheduled procedures for the 28-day follow-up visit as shown in the SoE have been finished.

It is anticipated that the total study duration for participants will be approximately 10 months to include screening, a 6-month treatment period, and a 3-month safety follow-up period. For participants continuing to the 6-month OLE, the 3-month safety follow-up period will occur at the end of the OLE or after early termination from the study for any reason. Participants will be screened within 28 days prior to enrollment for each group and will be randomly assigned to either AP-101 2500 mg IV or placebo, administered according to the SoE during the 6-month treatment period. After completing the treatment period, participants may continue in the study as part of the OLE if they have completed all study visits during the treatment period. All study treatment will be given at the study sites. PK results (exposure) and total body clearance of drug calculated after intravenous (IV) administration [CL] will be used as supporting data for any dose modifications, if available.

A participant is considered to have completed the study if Week 24 dosing and all scheduled procedures for the 28-day follow-up visit have been completed, as shown in the SoE (Table 2). The end of the study is defined as the date after the last participant completed 6 months of study treatments and completed any applicable follow-up.

### Justification for Dose

The proposed dose of 2500 mg AP-101 was selected based on a dose finding first-in-human (FIH) study in ALS patients (NCT03981536) and a toxicity study in cynomolgus monkeys (Study 8384444). This dose level has been confirmed as having an acceptable safety and tolerability profile after a single IV administration. The current study is designed with 2 loading doses on consecutive days, followed by repeat dosing once every 3 weeks for 6 months. Based on in silico modeling of PK data obtained from the FIH study, the dose schedule has been designed to enable patients to reach steady state as quickly as possible. The dose frequency is based on maintaining steady state throughout the treatment phase of the study.

Based on the preclinical, toxicology and clinical data available, a loading dose of 500 mg and 2500 mg given on consecutive days followed by once every 3 weeks dosing of 2500 mg IV is proposed. The dose level is based on preclinical efficacy studies performed in a mouse transgenic model of ALS which suggested that weekly intraperitoneal dosing of a mouse-chimeric α-miSOD1 at 3, 10, or 30 mg/kg (180-1800 mg dose equivalent, FDA 2005) showed efficacy in specific endpoints. Further, no overt toxicity (body weight loss) was observed in these animals.

The potential for AP-101 to produce toxicity or unwanted effects was evaluated in a 1-month study in cynomolgus monkeys (Study 8384444) which consisted of 2 IV injections separated by 2 weeks, each followed by an approximately 2-week post-dosing observation phase. The monkey is a relevant toxicology species because AP-101 binds to human and monkey SOD-1; AP-101 does not bind to rodent SOD1. No AP-101-related effects on toxicology or safety pharmacology parameters occurred at doses up to and including 400 mg/kg (24,000 mg for a 60-kg person), the highest dose tested. Therefore, 400 mg/kg is considered the no-observed-adverse-effect level (NOAEL).

In this 1-month study, monkeys were given bolus IV injections of 10, 60, or 400 mg/kg AP-101 on Study Days 1 and 15, with subsequent necropsy on Study Day 29. The top dose was selected to provide a multiple of the proposed human doses. Assessment of toxicity was based on mortality, clinical observations, body weight, ophthalmic observations, clinical pathology (hematology, coagulation, clinical chemistry, and urinalysis parameters) and anatomic pathology (organ weights, macroscopic and microscopic pathology).

There were no AP-101-related effects observed in any of the study parameters assessed. Serum AP-101 was measured at several time points and CSF AP-101 was measured at a single time point taken at necropsy, 2 weeks after second administration of AP-101. Serum exposure increased in a dose-proportional manner from 10 to 400 mg/kg and there was no evidence of accumulation with this dose interval. AP-101 was detectable in some 60- and 400-mg/kg monkey CSF samples collected at necropsy (approximately 2 weeks after the second IV dose). The relevant margins of safety based on the NOAEL from the 1-month monkey study are presented in Table 3. The proposed clinical starting dose of 100 mg is 235-fold lower than NOAEL established in the 1-month monkey study and is slightly lower than the lowest efficacy dose in the transgenic mouse efficacy study, while the highest proposed clinical dose, 2500 mg, is approximately 10-fold lower than the toxicology study NOAEL and slightly exceeds the maximal efficacy dose in the transgenic mouse efficacy study (as noted above, a chimeric molecule was tested in the transgenic mouse study). The proposed clinical study utilizes conservative dose escalation and appropriate safety monitoring. The results of the 1-month monkey study, taken together with the clinical study design, indicate that AP-101 has a safety profile that supports initiation of clinical development. Based on the PK observed in cynomolgus monkeys and humans, PK profiles were utilized for calculation of human exposure multiples; see Table 3.

TABLE 3
Margin of Safety for Intravenous Administration of AP-
101 Based on Administered Dose and Predicted Exposure

| | Dose | Dose | AUC₀₋₃₆₀ | Exposure |
|---|---|---|---|---|
| | (mg/kg) | Multiple^{a} | (μg*g/mL) | Multiple^{a} |
| Human Maximum | 42 | 9.5 x | 340,400,000 | 2.82 ^{a}x |
| (2500 mg) | | | | |
| Monkey NOAEL^{b} | 400 | | 961,000,000 | |

Abbreviations: AUC₀₋₃₃₆ = area under the plasma concentration time curve from time 0 to 336 hours; NOAEL = no-observed-adverse-effect level.
^{a}Dose multiple is the dose in animals/dose in humans based on mg/kg. Exposure multiple is calculated as the observed AUC0-336 in animals/predicted AUC0-336 in humans.
^{b}NOAEL determined in a 1-month repeat dose toxicity study (Study 8384444).

In the 6-month repeat dose study (Study 8384445), monkeys were given IV injections of 60 or 400 mg/kg AP-101 every 2 weeks for 26 weeks (a total of 13 doses). Assessment of toxicity was based on mortality, clinical observations, body weight, ophthalmic observations, clinical pathology (hematology, coagulation, clinical chemistry, and urinalysis parameters) and anatomic pathology (organ weights, macroscopic and microscopic pathology). There were no AP-101-related effects observed in any of the study parameters assessed, so the NOAEL was considered to be 400 mg/kg. Consistent with the absence of adverse effects in the 1- and 6-month monkey toxicology studies, there were no drug-related adverse events during the FIH study (AP101-01).

Dose Preparation Instructions for AP-101 for Injection, 100 mg Per Vial (5 mL á 20 mg/mL)

AP-101 drug product is administered as a slow intravenous (IV) infusion using a syringe pump for 500 mg doses and IV bags for 2500 mg doses. AP-101 for Injection is supplied for clinical trial use as a solution in a single-use glass vial. Further details are outlined in Examples 3 to 6.

### Study Population

Eligibility of study participants will be based on the results of assessments including screening, medical and disease history, physical examination, vital signs, clinical laboratory tests, Columbia-Suicide Severity Rating Scale (CSSR-S), ALS-Specific Quality of Life-Revised (ALSSQOL-SF), and ECG as described in the SoE. The detailed inclusion and exclusion criteria are listed in the Study Details of the Phase 2a study published under ClinicalTrials.gov Identifier: NCT05039099.

### Study Intervention

Study intervention is defined as any investigational intervention(s), marketed product(s), placebo, or medical device(s) intended to be administered to/used by a study participant according to the study protocol.

### Study Intervention(s) Administered

After randomization on Day 1, participants will receive a dose of either AP-101 500 mg or placebo, and on Day 2, another dose of either AP-101 2500 mg or placebo. Thereafter at each visit during the treatment period, AP-101 2500 mg or matching placebo will be administered via IV infusion to all participants over at least 60 minutes. Infusion duration may be increased, or the infusion may be stopped as deemed necessary based on the standard operation procedures of the study site, or if an infusion reaction is observed. However, IV administration needs to be completed within not more than 2 hours (120 minutes) from start of infusion. During the infusion and throughout the patient visit, the study site must have resuscitation equipment, emergency drugs, and appropriately trained staff available during the infusion period and for up to 6 hours after participants have completed their infusion. The actual start and stop time of the infusion will be recorded in the electronic data capture (EDC).

The AP-101 drug product is a monoclonal antibody formulated for IV administration. AP-101 for injection is supplied for clinical trial use as a solution in a single-use glass vial. The vial is manufactured to provide 100 mg of AP-101 at a 20 mg/mL concentration. In order to ensure complete withdrawal and delivery of the label amount of 100 mg of AP-101, vials contain an approximate 10% volume overfill. The drug product is filled into a single-use Type I glass vial.

### Preparation/Handling/Storage/Accountability

Vials of AP-101 are stable and should be stored under refrigerated conditions (2° to 8° C.). Pharmacy personnel will receive training and instructions for preparation of each dose of AP-101 IV solutions. Further guidance regarding preparation, handling, storage, and accountability is as follows:
1. The investigator or designee must confirm appropriate storage conditions have been maintained during transit for all study intervention received and any discrepancies are reported and resolved before use of the study intervention.
2. Only participants enrolled in the study may receive study intervention. Only study personnel may supply, prepare, or administer study intervention. All study intervention must be stored in a secure, environmentally controlled, and monitored (manual or automated) area in accordance with the labeled storage conditions with access limited to the investigator and authorized study personnel.
3. The investigator or authorized study personnel are responsible for study intervention accountability, reconciliation, and record maintenance (i.e., receipt, reconciliation, and final disposition records).
4. The investigator or designee is responsible for returning all unused medication to sponsor or its designee at the end of the study. In some cases, sites may destroy the material if, during the investigative site selection, the evaluator has verified and documented that the site has appropriate facilities and written procedures to dispose of clinical materials.

### Measures to Minimize Bias: Randomization and Blinding

This is a double-blind study. On Day 1, all participants will be centrally assigned to randomized study intervention using an interactive web-response system (IWRS). Study intervention will be dispensed at the study visits as summarized in the SoE. If an investigator, site personnel performing assessments, or participant is unblinded, the participant must be discontinued from the study but will be given the opportunity to enroll in the OLE. In cases where there are ethical reasons to have the participant remain in the study, the investigator must obtain specific approval from a sponsor clinical research physician for the participant to continue in the study.

The IWRS will be programmed with blind-breaking instructions. In case of an emergency, the investigator has the sole responsibility for determining if unblinding of a participants' intervention assignment is warranted. Participant safety must always be the first consideration in making such a determination. If a participant's intervention assignment is unblinded, the sponsor must be notified immediately after breaking the blind. The date and reason that the blind was broken must be recorded in the source documentation and case report form, as applicable.

Participants will be randomly assigned in a 2:1 ratio to receive study intervention. Investigators will remain blinded to each participant's assigned study intervention throughout the course of the study. In order to maintain this blind, an otherwise uninvolved and unblinded 3rd party will be responsible for the preparation of all blinded study intervention per each participant treatment assignment. The unblinded 3rd party personnel will receive training and instructions for preparation of each dose of study intervention, i.e., AP-101 or matching placebo solution for IV administration. The blinded study intervention will be administered to participant by blinded staff following randomization.

In the event of a Quality Assurance audit, the auditor(s) will be allowed access to unblinded study intervention records at the site(s) to verify that randomization/dispensing has been done accurately.

### Study Intervention Compliance

Study intervention will be administered under medical supervision by the investigator or designee. Documentation of treatment administration will occur at the site. The date and time of each dose administered will be recorded in the source documents and in the electronic case report form (eCRF).

### Concomitant Therapy

As standard of care agents, treatment with riluzole and/or edaravone is allowed where approved. Participants are required to be at a stable dose for at least 30 days prior to screening and remain on a stable dose throughout study. Riluzole or edaravone cannot be initiated during the placebo-controlled portion of the study; however, either can be initiated once the participant has been recruited to the planned open label extension. In general, concomitant medication should be avoided; however, over-the-counter medications may be administered at the investigator's discretion. (e.g., acetaminophen for treatment of headaches). If the need for concomitant medication (other than over-the-counter medications) arises, inclusion or continuation of the subject may be at the discretion of the investigator after consultation with the sponsor. Any medication used during the course of the study must be documented. Medically prescribed use of cannabis is allowed, per discretion of the investigator, during the study.

### Dose Modification

Dose levels, sampling schedule, timing of procedures (e.g., time of PK sample collection, ECGs) may be adjusted in view of emerging safety or PK data during the study. This is the first multiple dose study for AP-101 and therefore safety data would be the primary criteria for any dose modification. Safety data, in particular AEs, SAEs and adverse laboratory abnormalities, will be independently assessed by the investigator, sponsor and SRC and will be considered related to the study drug unless there is clear evidence that the event is not related. PK results (exposure) and total body clearance of drug calculated after IV administration [CL] will be used as supporting data for any dose modifications. After review of these data, an agreement on the appropriate dose modification will be made by the investigator, sponsor and SRC. Any dose modifications will be made only with the agreement of the investigator, sponsor and SRC.

### Premedication for Infusions

Premedication for the infusions is not planned. However, if an infusion reaction occurs, appropriate medication may be used as determined by the study investigator(s). If infusion reactions are observed, administration of acetaminophen, 500 to 1000 mg and/or an antihistamine may be administered orally or IV 30 to 60 minutes prior to the start of infusion for subsequent dose administrations. The decision to implement premedication for specific participants exhibiting infusion reactions will be made by the investigator, the sponsor, and the SRC, and recorded in the study documentation. Any premedications given will be documented as a concomitant therapy.

### Management of Infusion Reactions

There is a risk of infusion reaction with any biological agent; therefore, all participants should be monitored closely. Symptoms and signs that may occur as part of an infusion reaction include, but are not limited to: fever, chills, nausea, headache, bronchospasm, hypotension, angioedema, throat irritation, rash, pruritus, myalgia, and dizziness. In the event that a significant infusion reaction occurs, the following guidance should be followed:
The investigational product infusion should be slowed (for example, reduce infusion rate by 50% [for example, an infusion rate of 12 mL/hr becomes 6 mL/hr or slower]) or stopped, depending on the symptoms/signs present:
   if slowed, the infusion should be completed at the slower rate, as tolerated
   if determined by the investigator that the infusion should no longer continue, no further attempts to dose the participant should be made
Supportive care should be employed in accordance with the symptoms/signs.

### Discontinuation of Study Intervention and Participant Discontinuation/Withdrawal

Participants discontinuing from the treatment prematurely for any reason should complete AE and other follow-up procedures, see SoE.

### Discontinuation of Study Intervention

If the sponsor or investigator identifies a participant who did not meet enrollment criteria and was inadvertently enrolled, a discussion must occur between the sponsor and the investigator to determine if the participant may continue in the study. If both agree it is medically appropriate to continue, the investigator must obtain documented approval from the sponsor to allow the inadvertently enrolled participant to continue in the study with or without continued treatment with investigational product.

### F

Participants will be discontinued from the study in the following circumstances:
Enrollment in any other clinical study involving an investigational product or enrollment in any other type of medical research judged not to be scientifically or medically compatible with this study
I Participation in the study needs to be stopped for medical, safety, regulatory, or other reasons consistent with applicable laws, regulations, and good clinical practice (GCP)
If the investigator, after consultation with the sponsor-designated medical monitor, determines that a systemic hypersensitivity reaction has occurred related to study drug administration, the participant should be permanently discontinued from the investigational drug.
Investigator Decision
   the investigator decides that the participant should be discontinued from the study
   if the participant, for any reason, requires treatment with another therapeutic agent that has been demonstrated to be effective for treatment of the study indication, discontinuation from the study occurs prior to introduction of the new agent
Participant Decision
The participant, or legal representative, requests to be withdrawn from the study.
Termination of the study by the sponsor or regulatory authorities

Participants who withdraw from the study before completion of all study activities may be replaced at the discretion of the sponsor. If a clinically significant finding is identified (including, but not limited to changes from baseline in QT interval corrected using [Bazett's formula [QTcB] or Fridericia's formula [QTcF]]) after enrollment, the investigator or qualified designee will determine if the participant can continue in the study and if any change in participant management is needed. This review of the ECG printed at the time of collection must be documented. Any new clinically relevant finding should be reported as an AE. See the SoE for data to be collected at the time of intervention discontinuation and follow-up and for any further evaluations that need to be completed. Temporary discontinuation is not permitted for the current study.

Participant Discontinuation/Withdrawal from the Study

A participant may withdraw from the study:
at any time at his/her own request
at the request of his/her designee (for example, parents or legal guardian)
at the discretion of the investigator for safety, behavioral, compliance, or administrative reasons
if the participant becomes pregnant during the study
if enrollment in any other clinical study involving an investigational product or enrollment in any other type of medical research judged not to be scientifically or medically compatible with this study
if the participant, for any reason, requires treatment with another therapeutic agent that has been demonstrated to be effective for treatment of the study indication, discontinuation from the study occurs prior to introduction of the new agent.

Discontinuation is expected to be uncommon. At the time of discontinuing from the study, if possible, an early discontinuation visit should be conducted, as shown in the SoE. See SoE for data to be collected at the time of study discontinuation and follow-up and for any further evaluations that need to be completed. The participant will be permanently discontinued both from the study intervention and from the study at that time. If the participant withdraws consent for disclosure of future information, the sponsor may retain and continue to use any data collected before such a withdrawal of consent. If a participant withdraws from the study, he/she may request destruction of any samples taken and not tested, and the investigator must document this in the site study records.

If the sponsor or investigator identifies a participant who did not meet enrollment criteria and was inadvertently enrolled, then the participant should be discontinued from study treatment unless there are extenuating circumstances that make it medically necessary for the participant to continue on study treatment. If the investigator and the sponsor clinical research physician agree it is medically appropriate to continue, the investigator must obtain documented approval from the sponsor clinical research physician to allow the inadvertently enrolled participant to continue in the study with or without treatment with investigational product. Safety follow-up should be performed as outlined in the SoE, and in the sections as to safety assessments and AEs, infra.

### Lost to Follow-Up

A participant will be considered lost to follow-up if he or she repeatedly fails to return for scheduled visits and is unable to be contacted by the study site. Site personnel or designee are expected to make diligent attempts to contact participants who fail to return for a scheduled visit or were otherwise unable to be followed up by the site. Site personnel, or an independent third party, will attempt to collect the vital status of the participant within legal and ethical boundaries for all participants randomized, including those who did not get investigational product. Public sources may be searched for vital status information. If vital status is determined to be deceased, this will be documented, and the participant will not be considered lost to follow-up. Sponsor personnel will not be involved in any attempts to collect vital status information.

### Study Assessments and Procedures

Study procedures and their timing are summarized in the SoE.
Immediate safety concerns should be discussed with the sponsor immediately upon occurrence or awareness to determine if the participant should continue or discontinue study intervention.
Adherence to the study design requirements, including those specified in the SoE, is essential and required for study conduct.
All screening evaluations must be completed and reviewed to confirm that potential participants meet all eligibility criteria. The investigator will maintain a screening log to record details of all participants screened and to confirm eligibility or record reasons for screening failure, as applicable.

See the section as to laboratory tests, infra, listing the laboratory tests that will be performed for this study.

Table 4 provides a summary of the maximum number and volume of invasive samples for all sampling during the study.

TABLE 4
Protocol AP101-02 Blood Sampling Summary.

| | Blood | Number of | Total |
|---|---|---|---|
| | Volume per | Blood | Volume |
| Purpose | Sample (mL) | Samples | (mL) |
| Screening tests^{a} | 25 | 1 | 25 |
| Post-screening clinical | 15 | 13 | 195 |
| laboratory tests^{b} | | | |
| Pharmacokinetics | 5 | 23 | 115 |
| Serum biomarkers | 5 | 13 | 65 |
| Total | 50 | 50 | 590 |

^{a}Includes sampling for surface antigen of the hepatitis B virus (HBsAg) & hepatitis C virus (HCV) and clinical safety laboratory measures.
^{b}Additional samples, including for hypersensitivity analyses, may be drawn if needed for safety purposes.

In addition to the blood sampling volumes noted in Table 4, 3 post-screening CSF samples of 5 mL each will be collected, for a total of 15 mL. A screening CSF sample of 5 mL will also be drawn. Unless otherwise stated in this protocol, all samples collected for specified laboratory tests will be destroyed within 60 days of receipt of confirmed test results. Certain samples may be retained for a longer period, if necessary, to comply with applicable laws, regulations, or laboratory certification standards.

### Efficacy Assessments

Efficacy in this study will be measured as a decline in disease progression rate as measured by the ALSFRS-R, lung function via slow vital capacity (SVC) and/or pulse oximetry, muscle strength via hand-held dynamometry (HHD), quality of life by ALSSQOL-SF, and disease staging via King's staging tool.

Survival data may be confounded by the use of ventilation strategies. Therefore, a composite measurement of time to death, tracheostomy, or permanent ventilator use will be used.

The ALSFRS-R will be assessed at the visits and times specified in the SoE. The ALSFRS-R is a validated rating instrument for monitoring the progression of disability in patients with ALS. The ALSFRS-R scores correlate significantly with qualify of life as measured by the Sickness Impact Profile, indicated that the quality of function is a strong determinant of qualify of life in ALS. The ALSFRS-R has been demonstrated to predict survival. It measures 4 functional domains, including respiratory, bulbar function, gross motor skills, and fine motor skills. There are 12 questions, each scored 0 to 4 for a possible total score of 48 with higher score denoting better function (Cedarbaum et al., J. Neurol. Sci. 169 (1999), 13-21).

Upright SVC will be determined by performing 3-5 measures at the visits and times specified in the SoE, in accordance with criteria established by the American Thoracic Society and the European Respiratory Society (Goncalves de Barros et al., J. Bras. Pneumol 39 (2013), 317-322).

SpO2 stands for peripheral capillary oxygen saturation, an estimate of the amount of oxygen in the blood. Low SPO2 is associated with worsening respiratory function. SpO2 measurements will be performed as according to the SoE.

Muscle strength will be assessed through the use of HHD instruments at the visits and times specified in the SoE. Muscle strength is an important determinant of both function and ultimate survival in ALS. Quantitative muscle strength will be evaluated using HHD, which tests isometric strength of multiple muscles using standard positioning. Approximately 8 muscle groups will be examined bilaterally in both upper and lower extremities. A statistical analysis of 4 muscle groups can be used (HHD0) to accurately predict outcome.

Participants' ALS will be staged according to the King's staging tool, measured at the visits and times specified in the SoE (Roche et al., Brain 135 (Part 3) (2012), 847-852). This tool uses the following classifications of ALS development:
Stage 1: symptom onset (involvement of first region)
Stage 2A: diagnosis
Stage 2B: involvement of second region
Stage 3: involvement of third region
Stage 4A: need for gastrostomy
Stage 4B: need for non-invasive ventilation

This functional staging system ranks patients based upon their level of independence (Stages 1 and 2) and the need for significant interventions (feeding tube, respiratory support).

### Safety Assessments

Safety assessments will include clinical laboratory evaluations (hematology, clinical chemistry, endocrinology, and urinalysis), coagulation assessments, physical and neurological examinations, ECGs, vital signs, pregnancy tests, CSSR-S, quality of life, AE recording, and AE grading by National Cancer Institute Common Terminology Criteria for Adverse Events v. 5.0 (NCI CTCAE). In the event of a treatment-emergent hepatic abnormality additional selected clinical tests may be obtained and are detailed below in the section as to hepatic monitoring tests. Planned time points for all safety assessments are provided in the SoE.

A complete physical examination will include, at a minimum, assessments of the cardiovascular, respiratory, gastrointestinal and neurological systems. Height (screening only) and weight will also be measured and recorded. A brief physical examination will include, at a minimum, assessments of the skin, lungs, cardiovascular system, and abdomen (liver and spleen). Investigators should pay special attention to clinical signs related to previous serious illnesses.

A directed neurological examination will be performed by the investigator or designee. If abnormalities are noted at these time points, additional examinations should be performed until the participant has returned to baseline. The examiner should be familiar with the participant's baseline examination. Mandated elements of the examination include inspection for cranial nerves, tremor, extraocular movements, brachial and patellar deep tendon reflexes, finger-nose pointing, and Romberg sign. Work up for participants with clinically significant changes in neurological examination should be considered.

For each participant, vital signs, including blood pressure, pulse rate, and body temperature, measurements will be recorded. Single measurements will be taken at screening and the baseline visit. Supine triplicate blood pressure and pulse rate will be collected at all other time points. Body temperature to be obtained as single measurement. Pre-dose vital signs should be taken approximately 1 hour prior to the schedule dosing. At time points when orthostatic measurements are obtained, participants should be supine for at least 5 minutes and stand for approximately 2 minutes. When triplicate blood pressure or pulse rate measurements precede the orthostatic measurement, the last supine blood pressure or pulse rate measurement will be used for orthostatic calculations. If the participant feels unable to stand, supine vital signs only will be recorded. Unscheduled orthostatic vital signs should be assessed, if possible, during any AE of dizziness or posture-induced symptoms. Additional vital signs may be measured during each study period if warranted.

For each participant, a 12-lead digital ECG will be collected. Sites are recommended to conduct ECGs prior to any blood sample collection. Participants must be supine for approximately 5 to minutes before ECG collection and remain supine but awake during ECG collection. Electrocardiograms may be obtained at additional times, when deemed clinically necessary. All ECGs recorded should be stored at the investigational site. Electrocardiograms will be interpreted by a qualified physician (the investigator or qualified designee) at the site as soon after the time of ECG collection as possible, and ideally while the participant is still present, to determine whether the participant meets entry criteria at the relevant visit(s) and for immediate participant management, should any clinically relevant findings be identified. If a clinically significant quantitative or qualitative change from baseline is identified after enrolment, the investigator will assess the participant for symptoms (for example, palpitations, near syncope, syncope) to determine whether the participant can continue the study. The investigator or qualified designee is responsible for determining if any changes in participant management is needed and must document his/her review of the ECG printed at the time of evaluation form at least 1 of the replicate ECGs from each point. The machine-read ECG intervals and heart rate may be used for data analysis and report writing purposes unless a cardiologist overread of the ECGs is conducted prior to completion of the final study report (in which case the overread data would be used).

A list of clinical laboratory tests to be performed and to the SoE for the timing and frequency is provided, infra, in the corresponding section.
The investigator must review the laboratory results, document this review, and report any clinically relevant changes occurring during the study as an AE. The laboratory results must be retained with source documents unless a Source Document Agreement or comparable document cites an electronic location that accommodates the expected retention duration. Clinically significant abnormal laboratory findings are those which are not associated with the underlying disease, unless judged by the investigator to be more severe than expected for the participant's condition.
All laboratory tests with values considered clinically significantly abnormal during participation in the study or within 12 weeks after the last dose of study intervention should be repeated until the values return to normal or baseline or are no longer considered clinically significant by the investigator or medical monitor.
If such values do not return to normal/baseline within a period of time judged reasonable by the investigator, the etiology should be identified and the sponsor notified.
All protocol-required laboratory assessments must be conducted in accordance with the SoE, standard collection requirements, and laboratory manual.
If laboratory values from non-protocol specified laboratory assessments performed at an investigator-designated local laboratory require a change in participant management or are considered clinically significant by the investigator (e.g., SAE or AE or dose modification), then report the information as an AE.

Regarding coagulation, the prothrombin time (PT) w/INR test measures the length of time it takes for a blood clot to form in a sample of blood.

Regarding quality of life, the measurement of Health-Related Quality of Life is a valuable measure of therapeutic efficacy and will utilize the ALSSQOL-SF questionnaire. This assessment will be administered at time points displayed in the SoE. The ALSSQOL-SF is a 20-item instrument that measures overall quality of life (QOL) and 6 specific domains for individuals with ALS (Felgoise et al., Muscle Nerve 58 (2018), 646-654).

Suicidal ideation and behavior risk monitoring will be assessed using the C-SSRS, which is a semi-structured clinical interview that assess suicidal ideation severity, suicidal ideation intensity, and suicidal behavior. The C-SSRS will be assessed at the visits and times specified in the SoE.

### Adverse Events, Serious Adverse Events, and Investigational Product Complaints

The definitions of the following events can be found below in the sections as to adverse events:
AEs
SAEs
Investigational Product complaints (IPCs)

These events will be reported by the participant (or, when appropriate, by a caregiver, surrogate, or the participant's legally authorized representative). The investigator and any qualified designees are responsible for detecting, documenting, and recording events that meet these definitions and remain responsible for following up events that are serious, considered related to the study intervention or study procedures, or that caused the participant to discontinue the study. Care will be taken not to introduce bias when detecting events. Open-ended and non-leading verbal questioning of the participant is the preferred method to inquire about event occurrences. After the initial report, the investigator is required to proactively follow each participant at subsequent visits/contacts. All SAEs and AEs of special interest will be followed until resolution, stabilization, the event is otherwise explained, or the participant is lost to follow-up (as defined, supra). For investigational product complaints, the investigator is responsible for ensuring that follow-up includes any supplemental investigations as indicated to elucidate the nature and/or causality. The following Table 5 describes the timing, deadlines, and mechanism of collecting events.

TABLE 5
Timing, deadlines, and mechanism for collecting events.

| | | | | | |
|---|---|---|---|---|---|
| | | | Timing for | | |
| | | | Reporting to | | Back-Up |
| | Collection | Collection | Sponsor or | Mechanism for | Method of |
| Event | Start | Stop | Designee | Reporting | Reporting |

Adverse Event

| | | | | | |
|---|---|---|---|---|---|
| AE | Signing of | The final | As soon as | AE eCRF | N/A |
| | the informed | follow-up | possible upon | | |
| | consent form | visit | site awareness | | |
| | (ICF) | | | | |

Serious Adverse Event

| | | | | | |
|---|---|---|---|---|---|
| SAE and SAE | Signing of | The final | Within 24 hours | SAE eCRF or | SAE paper |
| updates - prior | the ICF | follow-up | of awareness | SAE paper | form |
| to start of | | visit | | form | |
| study | | | | | |
| intervention | | | | | |
| and deemed | | | | | |
| reasonably | | | | | |
| possibly | | | | | |
| related with | | | | | |
| study | | | | | |
| procedures | | | | | |
| SAE* and | Start of | Participation | Within 24 hours | SAE eCRF or | SAE paper |
| SAE updates - | intervention | in study has | of awareness | SAE paper | form |
| after start of | | ended | | form | |
| study | | | | | |
| intervention | | | | | |
| SAE* - after | After | N/A | Promptly | SAE paper | N/A |
| participant's | participant's | | | form | |
| study | study | | | | |
| participation | participation | | | | |
| has ended and | has ended | | | | |
| the | | | | | |
| investigator | | | | | |
| becomes | | | | | |
| aware | | | | | |

Pregnancy

| | | | | | |
|---|---|---|---|---|---|
| Pregnancy in | After the | 6 months | Within 24 hours | Pregnancy | SAE paper |
| female | start of study | after last | of learning of | eCRF or SAE | form |
| participants | intervention | dose | the pregnancy | eCRF or SAE | |
| and female | | | | paper form | |
| partners of | | | | | |
| male | | | | | |
| participants | | | | | |

Investigational Product Complaints

| | | | | | |
|---|---|---|---|---|---|
| PC associated | Start of | End of study | Within 24 hours | Investigational | N/A |
| with an SAE | study | intervention | of awareness | Product | |
| or might have | intervention | | | Complaint form | |
| led to an SAE | | | | | |
| PC not | Start of | End of study | Within 1 | Investigational | N/A |
| associated | study | intervention | business day of | Product | |
| with an SAE | intervention | | awareness | Complaint form | |
| Updated PC | — | — | As soon as | Originally | N/A |
| information | | | possible upon | completed | |
| | | | site awareness | Investigational | |
| | | | | Product | |
| | | | | Complaint form | |
| | | | | with all changes | |
| | | | | signed and | |
| | | | | dated by the | |
| | | | | investigator | |
| PC (if | Participation | N/A | Promptly | Investigational | |
| investigator | in study has | | | Product | |
| becomes | ended | | | Complaint form | |
| aware) | | | | | |

Abbreviations: AE = adverse event; eCRF = electronic case report form; ICF = informed consent document; PC = product complaint; SAE = serious adverse event.
*Serious adverse events, including death, caused by disease progression should not be reported unless the investigator deems them to be possibly related to study treatment.

### Adverse Events of Special Interest

Lumbar puncture (LP) to obtain CSF will be performed at several time points during this study. Headache can occur in up to 60% of patients who undergo LP, and backache can occur in up to 40% of patients. More serious adverse events are rare and include cerebral herniation, intracranial subdural hemorrhage, spinal epidural hemorrhage, and infection.

Hypersensitivity reaction may occur after administration of any monoclonal antibody. Acute hypersensitivity reactions, including infusion reactions or anaphylaxis, may occur during or within hours of the infusion. Sites should have appropriately trained medical staff and appropriate medical equipment available when study participants are receiving study drug. It is recommended that participants who experience a systemic hypersensitivity reaction be treated per national and international guidelines. In the case of generalized urticaria or anaphylaxis, additional blood samples should be collected as described below in the section as to recommended laboratory testing for hypersensitivity events”. Laboratory results are provided to the sponsor via the central laboratory.

Subjects should be monitored for hypotension, fever, chills, bronchospasm, angioedema, and other symptoms or signs of anaphylaxis. Infusion should be slowed or stopped, and reactions managed as indicated in study protocols. If such a reaction occurs, additional data describing each symptom should be provided to the sponsor in the eCRF. Delayed hypersensitivity reaction can develop days or weeks after infusion. Subjects should be monitored for fever, rash, arthralgia, myalgia, hematuria, changes in hematology, liver or renal tests. Delayed hypersensitivity reactions can be managed symptomatically and treated with corticosteroids. Subjects should not receive additional doses of AP-101 for any confirmed/suspected case of delayed hypersensitivity reaction to AP-101.

### Treatment of Overdose

For this study, any dose of AP-101 greater than 2500 mg of AP-101 within a 24-hour time period (±4 hours) will be considered an overdose. An overdose is not anticipated, as the study drug will be administered by a trained staff member. In the event of an overdose, the investigator should:
1. Contact the Medical Monitor immediately.
2. Closely monitor the participant for any AE/SAE and laboratory abnormalities until AP-101 can no longer be detected systemically (at least 100 days).
3. Document the quantity of the excess dose as well as the duration of the overdose in the eCRF.

Decisions regarding dose interruptions or modifications will be made by the investigator in consultation with the Medical Monitor based on the clinical evaluation of the participant.

### Pharmacokinetics (PK)

At the visits and times specified in the SoE, venous samples will be collected to determine the serum concentrations of AP-101. CSF sampling will also be collected at baseline and at defined timepoints during the study, according to the SoE. The sampling times may be modified at the discretion of the sponsor, based on review of interim PK data as they become available. Actual PK sample collection dates and times will be recorded.

Samples will be analyzed at a laboratory approved by the sponsor and stored at a facility designated by the sponsor. Concentrations of AP-101 will be assayed from serum and CSF using a validated immunoassay. Bioanalytical samples collected to measure study drug concentrations will be retained for a maximum of 1 year following last participant visit for the study. Table 4 presents a sampling summary for this study.

### Pharmacodynamics (PD)

Pharmacodynamic assessments in this study include changes in total SOD1, mSOD1, pNfH, and NfL as well as additional exploratory assessments. Levels of mSOD1 will be measured retrospectively from the CSF of participants at baseline and after at least 1 timepoint after treatment with AP-101. This is an exploratory endpoint that will be used to investigate whether there are correlations with levels of mSOD1 with response as well as whether treatment with AP-101 can reduce the levels of detectable mSOD1 in participants.

SOD1 and mSOD1 Assays

Levels of total SOD1 and misfolded SOD1 will be measured retrospectively from the CSF of participants at baseline and after at least 1 timepoint after treatment with AP-101. This is an exploratory endpoint that will be used to investigate whether there are correlations with levels of SOD1 with response as well as whether treatment with AP-101 can reduce the levels of detectable SOD1 in participants. Correlations with other exploratory biomarkers and endpoints will also be performed.

### Neurofilament Assays

Levels of pNfH and NfL will be measured from both CSF and serum at various timepoints during the 6-month study. This endpoint that will be used to determine the effects of AP-101 on pNfH and NfL levels. pNfH and NfL will also be examined for correlations with other exploratory biomarkers and endpoints.

### Genetics

Mandatory genetic testing for all participants to identify common variants associated with ALS will be conducted by a certified genetics laboratory. A blood sample for DNA isolation will be collected from participants.

### Biomarkers

Additional biomarkers will include creatinine and transcriptional and microRNA profiles from various biological fluids. These are exploratory biological markers of disease activity and will be examined at various timepoints during the study as displayed in the SoE.
This study will analyze biomarkers relevant to the mechanism of action for AP-101. Samples collected for these analyses may also be used to develop related research methods or to validate diagnostic tools or assays.
Additional samples may be collected during the study if warranted and agreed upon by the investigator and sponsor.

### Immunogenicity Assessments

Antibodies to AP-101 will be evaluated in serum samples collected from all participants according to the SoE. Additionally, serum samples should also be collected at the final visit from participants who discontinued study intervention or were withdrawn from the study. These samples will be tested by the sponsor or sponsor's designee. Serum samples will be screened for antibodies binding to AP-101 and the titer of confirmed positive samples will be reported. Other analyses may be performed to verify the stability of antibodies to AP-101 and/or further characterize the immunogenicity of AP-101. The detection and characterization of antibodies to AP-101 will be performed using a validated assay method by or under the supervision of the sponsor. All samples collected for detection of antibodies to study intervention will also be evaluated for AP-101 serum concentration to enable interpretation of the antibody data. Antibodies may be further characterized and/or evaluated for their ability to neutralize the activity of the study intervention(s). Samples may be stored for a maximum of 2 years (or according to local regulations) following the last participant's last visit for the study at a facility selected by the sponsor to enable further analysis of immune responses to AP-101.

### Statistical Considerations

Descriptive statistics, instead of hypothesis testing, will be used for statistical evaluation of efficacy, safety, and tolerability.

A maximum of 63 participants (21 with fALS and 42 with sALS) will be randomly assigned in a 2:1 randomization ratio to study intervention or placebo such that approximately 18 evaluable participants with fALS and 36 evaluable participants with sALS will complete the study. This sample size is based on clinical considerations and is not powered for hypothesis testing.

The populations depicted in Table 6 will be defined.

TABLE 6
Populations for Analyses

| Population | Description |
|---|---|
| Intent to Treat | All participants assigned to treatment, regardless of whether they take any |
| | doses of study treatment, or if they took the correct treatment. Participants |
| | will be analyzed according to the treatment group to which they were |
| | assigned. |
| Per-Protocol | All participants in the safety population who have completed the study, |
| (PP) | defined as completing Week 24 dosing and the |
| | 28-day follow-up visit. |
| Safety | All participants randomly assigned to study intervention and who take at |
| | least 1 dose of study intervention. Participants will be analyzed according |
| | to the intervention they actually received. |

All PK and PD (including neurofilament) analyses will be on performed on the safety population.

Statistical analysis of this study will be the responsibility of sponsor or its designee. All CIs will be given at a 2-sided 90% level. Any change to the data analysis methods described in the protocol will require an amendment only if it changes a principal feature of the protocol. Any other change to the data analysis methods described in the protocol, and the justification for making the change, will be described in the statistical analysis plan and the clinical study report. Additional exploratory analyses of the data will be conducted as deemed appropriate. The statistical analysis plan will be finalized prior to unblinding and it will include a more technical and detailed description of the statistical analyses described in this section. This section is a summary of the planned statistical analyses of the most important endpoints including primary and key secondary endpoints.

Statistical analysis of this study will be the responsibility of sponsor or its designee. Statistical analyses will be detailed in the statistical analysis plan. Safety analyses will be conducted for all dosed participants, whether or not they completed all protocol requirements as per the Safety Population. For continuous variables, summary statistics will include number of participants, mean, median, standard deviation, minimum, and maximum. Categorical endpoints will be summarized using number of participants, frequency, and percentages. A detailed description of participant disposition will be provided at the end of the study. All participants who discontinued from the study will be identified, and the extent of their participation in the study will be summarized. If known, a reason for their discontinuation will be provided. Participant demographics and baseline characteristics (age, gender, race, ethnicity, height, weight, and body mass index [BMI]) will be summarized by treatment. Summaries for safety and will also be provided.

The primary endpoints in this study are the incidence of AEs and SAEs, including immunogenicity, and the incidence of abnormalities in vital signs, clinical laboratory assessments, physical and neurological examinations, electrocardiograms, and changes in weight.

All treatment and protocol procedure AEs will be listed. Summary statistics for AEs and SAEs will be provided by treatment arm and sorted by system organ class and preferred term. Summaries will also be provided by relationship to study drug and maximum severity. AEs leading to discontinuation of study drug or of the study will be summarized. AEs will be summarized for the Safety Population. Abnormal vital signs, clinical laboratory assessments, physical and neurological examinations, electrocardiograms, and changes in weight will be provided as listings. The number of participants experiencing abnormalities and the number of abnormalities will be summarized using the Safety Population.

All secondary endpoints will be performed on the PK population. The PK analyses will only be performed for participants who received at least 1 full dose of study treatment and have baseline PK samples and at least 3 postbaseline evaluable PK samples. All serum PK samples will be utilized in a compartmental model to describe the PK for these patients. PK parameters will be reported with descriptive statistics which include: arithmetic mean, geometric mean, standard deviation, coefficient of variation (CV) %, geometric CV %, median, minimum, and maximum (Table 7).

TABLE 7
Pharmacokinetic Parameters

| Multiple- | |
|---|---|
| dose PK | |
| parameters | Definition |
| Cₘₐₓ | maximum drug concentration |
| Tₘₐₓ | time to maximum observed drug concentration, or first |
| | maximum observed drug concentration if this occurs at more |
| | than 1 time point |
| AUC₀₋ₜₗₐₛₜ | area under the plasma concentration-time curve from time 0 to |
| | the last quantifiable concentration |
| AUC₀₋ₜₐᵤ | area under the plasma concentration-time curve from time 0 to |
| | time tau (dosing interval) |
| V | apparent central volume of distribution |
| CL | total body clearance |
| Q | intercompartmental clearance |
| V₂ | peripheral compartment volume |
| t_{1/2} | elimination half-life |

During the 6-month treatment period, the level of AP-101 in the CSF will be summarized by treatment at each available treatment visit as well as the change from baseline at each visit using the safety population. Data will be summarized by treatment.

Both pNfH and NfL will be summarized by type (either CSF or plasma) for each available treatment visit as well as the change from baseline at each visit for both the ITT and per-protocol (PP) populations. Additional summaries by fALS status will be provided for the PP population. A statistical model will be employed to estimate the average change per month in pNfH and NfL in treated and placebo participants. The estimated change per month along with two-sided 90% confidence intervals will be included in the summaries. Plots showing the baseline levels of pNfH and NFL and their average change per month will be provided.

Total ALSFRS-R scores and change from baseline will be summarized at visits according to the SoE. A statistical model will be employed to estimate the average ALSFRS-R change per month in treated and placebo participants. This estimated change per month and two-sided 90% confidence intervals will be provided. Lung Function (SVC) or Equivalent Alternative, Muscle Strength (HHD), and Disease Staging (King's Staging Tool) scores will be summarized by visit and by change from baseline. Change from baseline in the King's staging tool will be summarized as the number of stages decreased since baseline. A Kaplan-Meier analysis will be performed comparing the treatment group to the placebo group. The 25th, 50th (median), and 75th quartiles will be presented along with their 90% confidence intervals, as well a plot of survival curves. The ALSSQOL-SF will be summarized by treatment visit. Total and misfolded SOD1 from the CSF will be summarized at each available visit as well as the change from baseline. An additional summary by fALS status will be provided. Two-sided 90% confidence intervals will be included in the summaries. Plots showing the baseline levels of SOD1 and mSOD1 and their change over time will be provided. All other biological markers of disease activity will be summarized at each available visit as well as the change from baseline. Two-sided 90% confidence intervals will be provided. All safety analyses will be made on the Safety Population and provided as listings. Subgroup analyses by fALS status (participants are either fALS or sALS) will be performed for various endpoints, including neurofilaments (NfL and pNfH) and measures of mSOD1 and SOD1. The subgroup analysis will be similar to the standard analysis in that it is also stratified by treatment. Any statistical modelling in the subgroup analyses will include a covariate term for fALS status and interaction term between fALS and treatment. Additional exploratory subgroup analyses may be performed.

### Clinical Laboratory Tests

Clinical laboratory tests are detailed in Table 8.

TABLE 8
Safety CLinical Laboratory Tests.
Safety Clinical Laboratory Tests

Hematology
Hematocrit
Hemoglobin
Erythrocyte count (RBC)
Mean cell volume
Mean cell hemoglobin
Mean cell hemoglobin concentration
Leukocytes (WBC)
Platelets
Differential WBC [Absolute counts and/or %]
Neutrophils
Lymphocytes
Monocytes
Eosinophils
Basophils
Urinalysis
Specific gravity
pH
Protein
Glucose
Ketones
Bilirubin
Urobilinogen
Blood
Nitrite
Clinical Chemistry
Sodium
Potassium
Bicarbonate [optional]
Chloride [optional]
Calcium [optional]
Phosphorus [optional]
Magnesium [optional except for QT studies or
investigational products with QT liability]
Glucose [choose random/fasting]
Blood urea nitrogen (BUN)
Uric acid [optional]
Total cholesterol [optional]
Total protein
Albumin
Total bilirubin
Alkaline phosphatase (ALP)
Aspartate aminotransferase (AST)
Alanine aminotransferase (ALT)
Creatinine
Gamma-glutamyl transferase (GGT) [optional]
Ethanol testing^{a,b}
Urine drug screen^{a,b}
Hepatitis B surface antigen^{a}
Hepatitis C antibody^{a}
HIV^{a}
Prothrombin time
Pregnancy test [if applicable]
FSH [if applicable]^{a}
Thyroid stimulating hormone [optional]
Abbreviations: FSH = follicle-stimulating hormone; HIV = human immunodeficiency virus; RBC = red blood cells; WBC = white blood cells.
^{a}Performed at screening only.
^{b}Urine drug screen and ethanol level may be repeated prior to admission to the clinical research unit and at other times indicated in the Schedule of Activities; urine drug screen is not conducted at the clinical research unit in Singapore

### Recommended Laboratory Testing for Hypersensitivity Events

Lab testing should be performed at the time of a Systemic Hypersensitivity Event. The management of the adverse event may warrant lab testing beyond that described below and should be performed as clinically indicated. Laboratory testing during a Systemic Hypersensitivity Event is not performed for diagnostic purposes. Its intent is several fold:
To help characterize and classify systemic hypersensitivity reactions
To meet regulatory expectations
To improve subsequent clinical management by helping to distinguish between the various mechanistic bases of anaphylaxis

Labs should be obtained in the presence of generalized urticaria or if anaphylaxis is suspected. After the subject has been stabilized, obtain a sample within 1-2 hours of the event; however, samples may be obtained as late as 12 hours after the event as analytes can remain altered for an extended period of time. Record the time at which the sample was collected. At the next regularly scheduled visit or after 4 weeks obtain a follow-up sample.

Clinical tests should include the following
Tryptase (If a tryptase sample is obtained more than 2 hours after the event (i.e., within 2-12 hours), or is not obtained because more than 12 hours have elapsed since the event, obtain urine for N-methylhistamine (NMH) testing. Note that for tryptase serum samples obtained within 2-12 hours of the event, urine NMH testing is performed in addition to tryptase testing. Collect the first void urine following the event. Obtain a follow-up urine for NMH testing at the next regularly scheduled visit or after 4 weeks, whichever is later.)
ADA and AP-101 concentration (PK)
Complement: C3, C3a and C5a
Cytokines: IL-6, IL-1B, IL-10 (or any cytokine panel that includes these 3 cytokines)

### Hepatic Monitoring Tests for Treatment-Emergent Abnormality

Selected tests as indicated in Table 9 may be obtained in the event of a treatment-emergent hepatic abnormality and may be required in follow-up with subjects.

TABLE 9
Hepatic Monitoring Tests.

Hepatic Hematology
Hemoglobin
Hematocrit
RBC
WBC
Neutrophils
Lymphocytes
Monocytes
Eosinophils
Basophils
Platelets
Hepatic Chemistry
Total bilirubin
Conjugated bilirubin
Alkaline phosphatase
ALT
AST
GGT
CPK
Haptoglobin
Hepatic Coagulation
Prothrombin time
Prothrombin time, INR
Hepatic Serologies^{a}
Hepatitis A antibody, total
Hepatitis A antibody, IgM
Hepatitis B surface antigen
Hepatitis B surface antibody
Hepatitis B core antibody
Hepatitis C antibody
Hepatitis E antibody, IgG
Hepatitis E antibody, IgM
Abbreviations: ALT = alanine aminotransferase; AST = aspartate aminotransferase; CPK = creatinine phosphokinase; GGT = gamma-glutamyl transferase; Ig = immunoglobulin; INR = international normalized ratio; RBC = red blood cell; WBC = white blood cell.
^{a}Reflex/confirmation dependent on regulatory requirements and/or testing availability.
Anti-nuclear Antibody^{a}
Alkaline Phosphatase Isoenzymes^{a}
Anti-smooth Muscle Antibody (or Anti-actin Antibody)^{a}

### Adverse Events (AE)

An AE is any untoward medical occurrence in a participant administered a pharmaceutical product and which does not necessarily have a causal relationship with the study intervention. An AE can therefore be any unfavourable and unintended sign (including an abnormal laboratory finding), symptom, or disease (new or exacerbated) temporally associated with the use of a medicinal (investigational) product, whether or not related to the medicinal (investigational) product.

### Events Meeting the AE Definition:

Any abnormal laboratory test results (hematology, clinical chemistry, or urinalysis) or other safety assessments (for example, ECG, radiological scans, vital signs measurements), including those that worsen from baseline, considered clinically significant in the medical and scientific judgment of the investigator (that is, not related to progression of underlying disease).
Exacerbation of a chronic or intermittent pre-existing condition including either an increase in frequency and/or intensity of the condition.
New conditions detected or diagnosed after study intervention administration even though they may have been present before the start of the study.
Signs, symptoms, or the clinical sequelae of a suspected drug-drug interaction.
Signs, symptoms, or the clinical sequelae of a suspected overdose of either study intervention or a concomitant medication. Overdose per se will not be reported as an AE/SAE unless it is an intentional overdose taken with possible suicidal/self-harming intent. Such overdose should be reported regardless of sequelae.
“Lack of efficacy” or “failure of expected pharmacological action” per se will not be reported as an AE or SAE. Such instances will be captured in the efficacy assessments. However, the signs, symptoms, and/or clinical sequelae resulting from lack of efficacy will be reported as AE or SAE if they fulfill the definition of an AE or SAE.

### Events not Meeting the AE Definition

Any clinically significant abnormal laboratory findings or other abnormal safety assessments which are associated with the underlying disease, unless judged by the investigator to be more severe than expected for the participant's condition.
The disease/disorder being studied or expected progression, signs, or symptoms of the disease/disorder being studied, unless more severe than expected for the participant's condition.
Medical or surgical procedure (for example, endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.

### SAE

If an event is not an AE per definition above, then it cannot be an SAE even if serious conditions are met (for example, hospitalization for signs/symptoms of the disease under study, death due to progression of disease).

SAE is defined as any untoward medical occurrence that, at any dose:
a. Results in death
b. Is life-threatening. The term ‘life-threatening’ in the definition of ‘serious’ refers to an event in which the participant was at risk of death at the time of the event. It does not refer to an event, which hypothetically might have caused death, if it were more severe.
c. Requires inpatient hospitalization or prolongation of existing hospitalization
   In general, hospitalization signifies that the participant has been admitted to hospital for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious.
   Hospitalization for elective treatment of a pre-existing condition that did not worsen from baseline is not considered an AE.
d. Results in persistent disability/incapacity
   The term disability means a substantial disruption of a person's ability to conduct normal life functions.
   This definition is not intended to include experiences of relatively minor medical significance such as uncomplicated headache, nausea, vomiting, diarrhea, influenza, and accidental trauma (for example, sprained ankle) which may interfere with or prevent everyday life functions but do not constitute a substantial disruption.
e. Is a congenital anomaly/birth defect
   Abnormal pregnancy outcomes (e.g., spontaneous abortion, fetal death, stillbirth, congenital anomalies, ectopic pregnancy) are considered SAEs.
f. Other situations:
   Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent one of the other outcomes listed in the above definition. These events should usually be considered serious.
   Examples of such events include invasive or malignant cancers, intensive treatment in an emergency room or at home for allergic bronchospasm, blood dyscrasias or convulsions that do not result in hospitalization, or development of drug dependency or drug abuse.

### Assessment of Intensity

The investigator will make an assessment of intensity for each AE and SAE reported during the study and assign it to 1 of the categories of the NCI-CTCAE Version 5.0 as outlined in Example 1.

### Assessment of Causality

The investigator is obligated to assess the relationship between study intervention and each occurrence of each AE/SAE. A “reasonable possibility” of a relationship conveys that there are facts, evidence, and/or arguments to suggest a causal relationship, rather than a relationship cannot be ruled out. The investigator will use clinical judgment to determine the relationship. Alternative causes, such as underlying disease(s), concomitant therapy, and other risk factors, as well as the temporal relationship of the event to study intervention administration will be considered and investigated. The investigator will also consult the IB in his/her assessment. For each AE/SAE, the investigator must document in the medical notes that he/she has reviewed the AE/SAE and has provided an assessment of causality. There may be situations in which an SAE has occurred and the investigator has minimal information to include in the initial report to sponsor or designee. However, it is very important that the investigator always make an assessment of causality for every event before the initial transmission of the SAE data to sponsor or designee. The investigator may change his/her opinion of causality in light of follow-up information and send a SAE follow-up report with the updated causality assessment. The causality assessment is one of the criteria used when determining regulatory reporting requirements.

The investigator is obligated to perform or arrange for the conduct of supplemental measurements and/or evaluations as medically indicated or as requested by sponsor or designee to elucidate the nature and/or causality of the AE or SAE as fully as possible. This may include additional laboratory tests or investigations, histopathological examinations, or consultation with other health care professionals.

### Examples 3 to 6 Related to AP-101 Formulations

### Methods

In certain methods, reference is made to Ph. Eur., which is the European Pharmacopoeia.

### Appearance (Clarity, Color and Visible Particles)

Appearance testing is performed to determine the clarity, color and visible particles. Clarity testing complies with Ph. Eur. <2.2.1. Clarity and Degree of Opalescence of Liquids>. The turbidimeter is used for clarity measurement. Color testing complies with Ph. Eur. <2.2.2. Degree of Coloration of Liquid>. The observational method is used for color measurement. Visible particle testing complies with Ph. Eur. <2.9.20. Particulate Contamination: Visible Particles>. The observational method is used for visible particle detection.

### Osmolality

Testing for osmolality complies with Ph. Eur. 2.2.35. The osmolality is determined indirectly by the measurement of the depression of the freezing point of the solution.

### Extractable Volume

Extractable volume testing complies with Ph. Eur. 2.9.17. The volumetric method is used for the extractable volume test.

### Particulate Matter

The particulate matter testing complies with Ph. Eur. 2.9.19. The number of subvisible particles is determined based on light obscuration. When particles in liquid pass a narrow detection channel, incident light perpendicular to the flow direction of the liquid is weakened due to obstruction from particles, leading to a reduction of signal output by the sensor. The change of signal is related to the sectional area.

### Sterility

The sterility test for drug product is established based on Ph. Eur. 2.6.1. The membrane filtration assay is used where the contents of the containers to be tested are filtered through two 0.45 μm membranes; any microorganisms present will be retained on the membranes. Each membrane is flushed with an established volume of rinse solution. Two kinds of culture media are used to incubate the membranes: Fluid Thioglycollate Medium (FTM) and Soybean-Casein Digest (TSB). FTM is transferred into one canister and TSB into another canister. The FTM and TSB canisters are incubated for 14 days (FTM at 30-35° C. and TSB at 20-25° C.). No growth of microorganisms indicates the tested samples are sterile.

Further analytical procedures used to control parameters of the drug product such as measurement of pH, and protein concentration, e.g., by using a UV-visible spectrophotometer, size-exclusion chromatography (SEC), capillary electrophoresis sodium dodecyl sulfate in reduced form (Reduced CE-SDS), capillary Isoelectric Focusing (cIEF), and fluorescence resonance energy transfer (FRET)-binding were performed in accordance with the official standards for quality control as set out in the Ph. Eur. and as described in standard literature such as Mikkelsen and Cortón “Bioanalytical Chemistry”, Second Edition (2016), Hoboken, New Jersey: John Wiley & Sons, Inc. dent, ISBN 9781119057703 (pdf); Schiel et al., “State-of-the-Art and Emerging Technologies for Therapeutic Monoclonal Antibody Characterization Volume 2. Biopharmaceutical Characterization: The NISTmAb Case Study”, ACS Symposium Series 1201 (2015), ISBN 978-0-8412-3029-3, for example Chapter 5 by Michels et al.; Kulkarni et al., “Essential chemistry for formulators of semisolid and liquid dosages”, Academic Press (2016), ISBN 978-0-12-801024-2, Chapter 11—Miscellaneous Physical, Chemical, and Microbiological Test Methods, pages 193-221; or on the homepage of Protagen Protein Services (https://www.protagenproteinservices.com/biopharmaceuticals/antibodies).

### Example 3: Development of a Stable Antibody Formulation for Antibody AP-101

Formulation development of AP-101 (herein referred to as drug substance) included studies designed to select a buffer system, excipients and surfactant to stabilize the protein. The formulation was developed to prevent product loss, as well as minimize the purity and bioactivity decline against stresses encountered during production, storage, shipping and handling.

A buffer system study was performed to determine the optimal buffer system for antibody formulation. Based on a pH screening study, 20 mM L-histidine/L-histidine monohydrochloride buffer, pH 6.0 was chosen as the final buffer system.

Different types of excipients, including disaccharides (sucrose, trehalose and sorbitol), amino acids (L-arginine-hydrochloride and L-methionine) and salt (NaCl) were evaluated through excipients studies. Samples were incubated at 25° C. and 40° C. for up to 4 weeks. The thermal stability, formation of insoluble aggregates and purity were monitored. Overall, sucrose and L-methionine with the concentration of 8% (w/v) and 0.1% (w/v), respectively, were chosen as the optimal excipients for AP-101 formulation, as they were shown to minimize the formation of low molecular weight and acidic species, thus retaining product purity.

In a surfactant type and strength screening study, four different polysorbate 80 (PS 80) concentrations (0.005%, 0.010%, 0.020% and 0.050% (w/v)) and two different poloxamer 188 concentrations (0.05% and 0.10% (w/v)) were tested. Number of sub-visible particles and purity were evaluated. A 0.02% (w/v) PS 80 concentration was chosen as the surfactant strength as sub-visible particle formation was effectively suppressed and acceptable stability was shown.

The final formulation developed is AP-101 at a target concentration of 20 mg/mL in 20 mM L-histidine/L-histidine monohydrochloride buffer, 8% (w/v) sucrose, 0.1% (w/v) L-methionine and 0.02% (w/v) PS 80 at pH 6.0. The excipients and their stabilizing role used in the formulation of AP-101 are summarized in Table 10.

TABLE 10
Role of excipients in the drug substance

| Excipient | Contribution to Stability |
|---|---|
| L-histidine | Buffer |
| L-histidine monohydrochloride | Buffer |
| Sucrose | Tonicity modifier |
| L-methionine | Antioxidant |
| Polysorbate 80 | Surfactant |

### Example 4: Manufacturing of the Final Drug Product Comprising Antibody AP-101

Manufacturing of the drug product was performed by drug substance thawing (bulk product), pooling and mixing, sterile filtration, aseptic filling, stoppering, and capping. Optionally, visual inspection, UV printing, bulk packaging, and storage at 2-8° C. was performed. In particular, frozen drug substance stored in 10 L polycarbonate bottles was thawed at room temperature (18-24° C.) in a room protected from light. After complete thawing, the drug substance was pooled into a 100 L mixing bag and stirred at an appropriate speed≤250 rpm, so that movement was observed without generating foam. The mixing time is controlled at >15 minutes. Prior to sterile filtration, samples were taken for pH, osmolality, protein concentration, endotoxin and bioburden.

Sterile filtration was chosen as the method to obtain the sterile drug product and was performed by aseptically filtering the bulk drug substance under laminar air flow (LAF) with a Grade C background via a peristaltic pump using two series connected sterile filters (0.22, polyvinylidene fluoride membrane) into a sterile 50 L single use bag. Prior to and after sterile filtration, filter integrity testing was performed on both filters.

Afterwards, the drug product was filled aseptically into 8 mL sterile glass vials using a filling and closing machine connected with a peristaltic pump system. All filling components were autoclaved and aseptically assembled. The compatibility of the drug product with the contacting components on the filling line, the impact of shear stress caused by the peristaltic pump, and stability under light exposure have been evaluated to mitigate potential adverse impact on product quality attributes during manufacturing. Based on a shear stress study and prior manufacturing knowledge, the drug product was filled with a peristaltic pump at a speed≤350 rpm. The filling pump with an automatic filler and stoppering unit were enclosed within a Restricted Access Barrier System (RABS) to fully enclose the aseptic process and provide a Grade A environment.

In the next step, vials were automatically stoppered with 20 mm rubber stoppers inside the RABS unit. The stoppers were steam sterilized at 122° C. for 30 minutes. Capping was performed by transferring the stoppered vials to the capping machine via conveyor belt under laminar air flow protection. The stoppered vials are capped with 20 mm plastic aluminum flip-off caps. The caps are steam sterilized at 122° C. for 30 minutes. Thus, the container closure system for the drug product is an 8 mL Type I glass vial sealed with a 20 mm rubber stopper and a 20 mm flip-off cap.

Afterwards, a manual 100% visual inspection was performed on the filled vials by production personnel, followed by a statistically based AQL inspection by Quality Assurance. Release and stability samples were taken after visual inspection. Part of the lot number was UV ink-printed on the side of cap after visual inspection process. A manual 100% check was performed on the printed number.

The filled drug product vials were then bulk packaged and labelled. Bulk packaged drug product vials were stored at 2-8° C.

### Example 5: Compatibility of the AP-101 Formulation with Clinical in-Use Materials

Stability data were collected to confirm the stability of the drug product dosing solution in the clinical trial setting. The compatibility with polyvinyl chloride IV lines and bags, polypropylene syringes, PVC infusion set, polyolefin IV bags, and fluorinated ethylene propylene (FEP)-indwelling catheter were assessed. Two concentrations 20 mg/mL (undiluted drug product), and 5 mg/mL were assessed. Normal saline was used as a diluent in the 5 mg/mL conditions. Solutions were held in infusion containers (syringe or IV bag) at 2˜8° C. for 24 hours then held at room temperature for 6 hours, and finally infused over a period of 2 hours (defined as T30H). Samples were tested at T0 and T30H. Some of the results are shown in Table 11 to Table 13.

TABLE 11
In-use Compatibility Study Results (1)

| | Target | | | | | | | Non-Reduced | |
|---|---|---|---|---|---|---|---|---|---|
| | Protein | SEC-HPLC | | | | | | CE-SDS | |
| | Concentration | Main Peak % | | HMW % | | LMW % | | Purity % | |
| Dose | (mg/mL) | T0 | T30 H | T0 | T30 H | T0 | T30 H | T0 | T30 H |
| Control sample | 5 | 98.2 | 98.4 | 1.1 | 0.9 | 0.7 | 0.7 | 98.0 | 97.5 |
| Control sample | 20 | 98.4 | 98.4 | 0.9 | 0.9 | 0.6 | 0.7 | 98.0 | 97.6 |
| Syringe sample | 5 | 98.3 | 98.3 | 1.0 | 1.0 | 0.7 | 0.7 | 97.9 | 97.7 |
| Syringe sample | 20 | 98.4 | 98.4 | 1.0 | 1.0 | 0.7 | 0.7 | 97.9 | 97.6 |
| IV bag sample | 20 | 98.4 | 98.3 | 1.0 | 1.0 | 0.7 | 0.7 | 98.0 | 97.6 |

TABLE 12
In-use Compatibility Study Results (2)

| | Target | | | | | | | | |
|---|---|---|---|---|---|---|---|---|---|
| | Protein | cIEF | | | | | | | |
| | Concentration | pI | | Main peak % | | Acidic peak % | | Basic peak % | |
| Dose | (mg/mL) | T0 | T30 H | T0 | T30 H | T0 | T30 H | T0 | T30 H |
| Control sample | 5 | 7.6 | 7.6 | 70.7 | 68.5 | 23.6 | 25.5 | 5.7 | 6.0 |
| Control sample | 20 | 7.7 | 7.7 | 69.0 | 68.8 | 25.1 | 24.5 | 5.8 | 6.7 |
| Syringe sample | 5 | 7.6 | 7.6 | 71.1 | 69.2 | 23.8 | 25.1 | 5.1 | 5.7 |
| Syringe sample | 20 | 7.7 | 7.7 | 69.8 | 69.3 | 24.1 | 24.9 | 6.1 | 5.8 |
| IV bag sample | 20 | 7.7 | 7.7 | 70.2 | 68.2 | 24.1 | 25.0 | 5.7 | 6.8 |

TABLE 13
In-use Compatibility Study Results (3)

| | Target | | | Protein | | | | | |
|---|---|---|---|---|---|---|---|---|---|
| | Protein | | | Concentration | | | | | |
| | Concentration | Appearance | | (mg/mL) | | pH | | Osmolality | |
| Dose | (mg/mL) | T0 | T30 H | T0 | T30 H | T0 | T30 H | T0 | T30 H |
| Control sample | 5 | CSF¹ | CSF | 5.2 | 5.2 | 6.1 | 6.0 | 285 | 285 |
| Control sample | 20 | CSF | CSF | 20.3 | 20.8 | 5.9 | 6.0 | 301 | 301 |
| Syringe sample | 5 | CSF | CSF | 5.5 | 5.5 | 6.0 | 6.0 | 289 | 290 |
| Syringe sample | 20 | CSF | CSF | 20.9 | 20.9 | 5.9 | 6.0 | 301 | 309 |
| IV bag sample | 20 | CSF | CSF | 20.9 | 20.7 | 6.0 | 6.0 | 303 | 299 |

¹CSF: colorless, slightly opalescent liquid and free of visible particle

The data indicate that AP-101 is compatible with the clinical in-use materials evaluated. The product diluted with normal saline at concentrations of 5 mg/mL and 20 mg/mL was stable at 2-8° C. for 24 hours, then at room temperature for up to 6 hours and infusion for 2 hours. No substantial changes in the appearance, protein concentration, pH, osmolality, SEC, non-reduced CE-SDS purity, or cIEF purity were observed. Results show that the drug product solution after preparation for dosing is stable for at least 24 hours at refrigerated temperature and 6 hours at room temperature.

### Example 6: Stability of AP-101 Formulation

Representative batches of drug product were evaluated for stability to assure pharmaceutical performance and strength for the duration of clinical trials. Long-term stability studies (Real-time studies) were performed at 5° C.±3° C. and sample analysis was performed at T0, at 1 month, 3 months, 6 months, 9 months, 12 months, 18 months, and 24 months. Furthermore, accelerated stability studies were performed at 25° C.±2° C. and 60 RH±5% RH (relative humidity) at T0, at 1 month, 3 months, and 6 months. In addition, stability was analyzed under stress conditions at 40° C.±2° C. and 75% RH±5% RH.

In this context, it is noted that
3 months real time data support a 12-months shelf life
6 months real time data support a 18-months shelf life
9 months real time data support a 21-months shelf life
12 months real time data support a 24-months shelf life
24 months real time data support a 36-months shelf life
36 months real time data supports a 48-month shelf-life
44 months real time data supports a 56-month shelf life
53 months real time data supports a 65-month shelf-life

The results are summarized in Table 14 to Table 17. The data revealed that the AP-101 formulation is long-term stable. There is no evidence of any significant physical or chemical changes in the drug product at any of the storage conditions employed. Since the formulation has been shown to be stable for 24 months, the shelf-life is expected to be at least 36 months. Stability was even confirmed for 53 months and thus, the shelf-life is expected to be up to 65 months.

TABLE 14
Real-time Stability Data for Clinical Lot 201809060 at 5 ± 3° C. (Inverted) through 24 months

| | | | 1 | 3 | 6 | 9 | 12 | 18 | 24 |
|---|---|---|---|---|---|---|---|---|---|
| Test | | T0 | Month | Month | Month | Month | Month | Month | Month |
| Appearance | Clarity | 6 NTU | 6 NTU | 7 NTU | 6 NTU | 6 NTU | 6 NTU | 6 NTU | 6 NTU |
| | Color | No more | No more | No more | No more | No more | No more | No more | No more |
| | | color than | color than | color than | color than | color than | color than | color than | color than |
| | | Y7 | Y6 | Y6 | Y7 | Y7 | Y7 | Y7 | Y7 |
| | Visible | Essentially | Essentially | Essentially | Essentially | Essentially | Essentially | Essentially | Essentially |
| | Particles | free of | free of | free of | free of | free of | free of | free of | free of |
| | | visible | visible | visible | visible | visible | visible | visible | visible |
| | | particles | particles | particles | particles | particles | particles | particles | particles |
| pH | | 6.0 | 6.0 | 6.0 | 6.0 | 6.0 | 6.0 | 6.0 | 6.0 |
| Osmolality | | 307 | 305 | 302 | 300 | 300 | 301 | 300 | 302 |
| | | mOsmol/kg | mOsmol/kg | mOsmol/kg | mOsmol/kg | mOsmol/kg | mOsmol/kg | mOsmol/kg | mOsmol/kg |
| Extractable Volume | | No less than | NT² | NT² | NT² | NT² | No less than | NT² | No less than |
| | | 5.0 mL | | | | | 5.0 mL | | 5.0 mL |
| Particulate | ≥10 μm | 174 particles/ | 79 particles/ | 38 particles/ | 32 particles/ | 16 particles/ | 11 particles/ | 11 particles/ | 15 particles/ |
| | | vial | vial | vial | vial | vial | vial | vial | vial |
| Matter | ≥25 μm | 0 particles/ | 4 particles/ | 1 particles/ | 0 particles/ | 0 particles/ | 1 particles/ | 0 particles/ | 1 particles/ |
| | | vial | vial | vial | vial | vial | vial | vial | vial |
| Quantity | Concentration | 20.52 | 20.32 | 20.54 | 20.59 | 20.39 | 20.39 | 20.44 | 20.53 |
| | | mg/mL | mg/mL | mg/mL | mg/mL | mg/mL | mg/mL | mg/mL | mg/mL |
| | % of the | 102.6% | 101.6% | 102.7% | 103.0% | 102.0% | 102.0% | 102.2% | 102.7% |
| | claim label | | | | | | | | |
| SEC | Main Peak | 99.1% | 99.0% | 98.9% | 98.8% | 98.8% | 98.8% | 98.7% | 98.6% |
| | (monomer) | | | | | | | | |
| | HMWS | 0.9% | 0.9% | 1.0% | 1.1% | 1.1% | 1.1% | 1.2% | 1.2% |
| | LMWS | 0.1% | 0.1% | 0.1% | 0.1% | 0.1% | 0.1% | 0.1% | 0.2% |
| Reduced | HC + LC | 97.1% | 97.3% | 97.3% | 97.1% | 97.5% | 97.3% | 97.0% | 96.6% |
| CE-SDS | | | | | | | | | |
| cIEF | Main peak | 67.8% | 68.1% | 68.3% | 68.6% | 68.8% | 67.6% | 67.4% | 67.2% |
| | Acid Variants | 25.4% | 25.5% | 25.4% | 25.4% | 25.9% | 26.3% | 26.5% | 27.0% |
| | Basic Variants | 6.8% | 6.5% | 6.3% | 6.0% | 5.3% | 6.1% | 6.0% | 5.8% |

| | | | | | | | | |
|---|---|---|---|---|---|---|---|---|
| FRET-Binding | 102% | 97% | 100% | 104% | 99% | 98% | 103% | 101% |
| (relative potency) | | | | | | | | |
| Sterility | No | NT¹ | NT¹ | NT¹ | NT¹ | No | NT¹ | No |
| | growth | | | | | growth | | growth |

¹NT = not tested.

TABLE 15
Real-time Stability Data for Clinical Lot 201809060 at 5 ±
3° C. (Inverted) - 30 months through 53 months

| Test | | 30 Month | 36 Month | 44 Month | 53 Month |
|---|---|---|---|---|---|
| Appearance | Clarity | 6 NTU | 6 NTU | 6 NTU | 7NTU |
| | Color | No more color | No more color | No more color | No more color |
| | | than Y6 | than Y6 | than Y6 | than Y6 |
| | Visible Particles | Essentially | Essentially | Essentially | Essentially |
| | | free of visible | free of visible | free of visible | free of visible |
| | | particles | particles | particles | particles |
| pH | | 6.1 | 6.0 | 6.0 | 6.0 |
| Osmolality | | 303 | 303 | 303 | 302 |
| | | mOsmol/kg | mOsmol/kg | mOsmol/kg | mOsmol/kg |
| Extractable Volume | | NT² | No less than | No less than | No less than |
| | | | 5.0 mL | 5.0 mL | 5.0 mL |
| Particulate Matter | ≥10 μm | 26 | 17 | 25 | 16 |
| | | particles/vial | particles/vial | particles/vial | particles/vial |
| | ≥25 μm | 1 | 1 | 1 | 0 |
| | | particles/vial | particles/vial | particles/vial | particles/vial |
| Quantity | Concentration | 20.27 | 20.32 | 20.31 | 20.51 |
| | | mg/mL | mg/mL | mg/mL | mg/mL |
| | % of the claim label | 101.4% | 101.5% | 101.6% | 102.5% |
| SEC | Main Peak (monomer) | 98.6% | 98.5% | 98.4% | 97.4% |
| | HMWS | 1.2% | 1.2% | 1.2% | 1.3% |
| | LMWS | 0.2% | 0.3% | 0.4% | 1.3% |
| Reduced CE-SDS | HC + LC | 96.9% | 96.9% | 96.7% | 96.6% |
| cIEF | Main peak | 66.5% | 67.4% | 66.5% | 66.5% |
| | Acid Variants | 27.7% | 27.4% | 29.1% | 29.5% |
| | Basic Variants | 5.7% | 5.2% | 4.4% | 5.0% |

| | | | | |
|---|---|---|---|---|
| FRET-Binding | 92% | 104% | 107% | 104% |
| (relative potency) | | | | |
| Sterility | NT¹ | No growth | No growth | No growth |

²NT = not tested.

TABLE 16
Accelerated Stability Data for Clinical Lot 201809060 at 25 ± 2° C./60 ± 5% RH (Inverted)

| | | Acceptance | | | | |
|---|---|---|---|---|---|---|
| Test | | Criteria | T0 | 1 Month | 3 Month | 6 Month |
| Appearance | Clarity | ≤20 NTU | 6 NTU | 6 NTU | 6 NTU | 6 NTU |
| | Color | No more | No more | No more | No more | No more |
| | | color than | color than | color than | color than | color than |
| | | Y4 | Y7 | Y6 | Y6 | Y6 |
| | Visible | Essentially | Essentially | Essentially | Essentially | Essentially |
| | Particles | free of | free of | free of | free of | free of |
| | | visible | visible | visible | visible | visible |
| | | particles | particles | particles | particles | particles |
| pH | | 6.0 ± 0.5 | 6.0 | 6.0 | 6.0 | 6.0 |
| Osmolality | | 310 ± 50 | 307 | 309 | 303 | 302 |
| | | mOsmol/kg | mOsmol/kg | mOsmol/kg | mOsmol/kg | mOsmol/kg |
| Particulate | ≥10 μm | ≤6000 particles/ | 174 particles/ | 45 particles/ | 30 particles/ | 59 particles/ |
| Matter | | vial | vial | vial | vial | vial |
| | ≥25 μm | ≤600 particles/ | 0 particles/ | 0 particles/ | 0 particles/ | 2 particles/ |
| | | vial | vial | vial | vial | vial |
| Quantity | Conc. | report result | 20.52 | 20.29 | 20.52 | 20.55 |
| | | | mg/mL | m/mL | mg/mL | mg/mL |
| | % of the | not less than | 102.6% | 101.5% | 102.6% | 102.8% |
| | claim label | 85.0% and not | | | | |
| | | more than | | | | |
| | | 115.0% of the | | | | |
| | | label claim | | | | |
| SEC | Main Peak | ≥92.0% | 99.1% | 98.0% | 97.6% | 96.1% |
| | (monomer) | | | | | |
| | HMWS | ≤5.0% | 0.9% | 1.1% | 1.1% | 1.4% |
| | LMWS | report result | 0.1% | 0.9% | 1.3% | 2.5% |
| Reduced | HC + LC | ≥90.0% | 97.1% | 97.6% | 96.7% | 95.4% |
| CE-SDS | | | | | | |
| cIEF | Main Peak | ≥45.0% | 67.8% | 66.5% | 61.7% | 56.7% |
| | Acid Variants | ≤45.0% | 25.4% | 27.6% | 32.6% | 39.2% |
| | Basic Variants | ≤15.0% | 6.8% | 5.9% | 5.7% | 4.2% |

| | | | | | |
|---|---|---|---|---|---|
| FRET-Binding | 60%-140% | 102% | 96% | 109% | 95% |
| (relative potency) | | | | | |

TABLE 17
Stress Stability Data for Clinical Lot 201809060 at
40 ± 2° C./75 ± 5% RH (Inverted)

| Test | | Acceptance Criteria | T0 | 1 Week |
|---|---|---|---|---|
| Appearance | Clarity | ≤20 NTU | 6 NTU | 6 NTU |
| | Color | No more color | No more color | No more color |
| | | than Y4 | than Y7 | than Y7 |
| | Visible | Essentially | Essentially | Essentially |
| | Particles | free of visible | free of visible | free of visible |
| | | particles | particles | particles |
| pH | | 6.0 ± 0.5 | 6.0 | 6.0 |
| Osmolality | | 310 ± 50 | 307 | 305 |
| | | mOsmol/kg | mOsmol/kg | mOsmol/kg |
| Particulate Matter | ≥10 μm | ≤6000 particles/ | 174 particles/ | 380 particles/ |
| | | vial | vial | vial |
| | ≥25 μm | ≤600 particles/ | 0 particle/ | 16 particle/ |
| | | vial | vial | vial |
| Quantity | Concentration | report result | 20.52 | 20.24 |
| | | | mg/mL | mg/mL |
| | % of the claim | not less than | 102.6% of the | 101.2% of the |
| | label | 85.0% and not more | claim label | claim label |
| | | than 115.0% of the | | |
| | | label claim | | |
| SEC | Main Peak | ≥92.0% | 99.1% | 98.8% |
| | (monomer) | | | |
| | HMWS | ≤5.0% | 0.9% | 1.0% |
| | LMWS | report result | 0.1% | 0.2% |
| Reduced CE-SDS | HC + LC | ≥90.0% | 97.1% | 96.8% |
| cIEF | Main Peak | ≥45.0% | 67.8% | 61.8% |
| | Acid Variants | ≤45.0% | 25.4% | 30.8% |
| | Basic Variants | ≤15.0% | 6.8% | 7.4% |

| | | | |
|---|---|---|---|
| FRET-Binding | 60%-140% | 102% | 93% |
| (relative potency) | | | |

### Example 7: N-Linked Glycosylation of the Heavy Chain, N-Terminal Pyro-Glutamic Acid Modified from N-Terminal Glutamine, and C-Terminal Lysine Clipping of the Heavy Chain are the Major Post-Translational Modifications of AP-101

Antibody AP-101 is produced in the CHO-K1 cell line (ATCC No. CCL 61) and obtained from the cell culture after culturing in a large-scale production bioreactor.

Liquid Chromatography with tandem mass spectrometry (LC-MS/MS) analysis of fragments of AP-101 obtained from Lys-C and trypsin sequential digestion as well as free sulfhydryl analysis was used to identify post-translational modifications of AP-101. The characterization of antibody-based therapeutics via LC-MS analysis is a standard procedure and can be performed by a skilled artisan; see for example Robotham and Kelly, Approaches to the Purification, Analysis and Characterization of Antibody-Based Therapeutics (2020), 1-33.

Glutamine at N-terminal modified as pyro-glutamic acid and C-terminal lysine clipping of the heavy chain have been identified as the major post-translational modifications and N303 has been identified as N-glycosylation site. In addition, small ratio modifications such as methionine oxidation, asparagine deamidation and asparagine succinimide formation have been observed. The results of LC-MS/MS analysis are summarized in Table 18.

TABLE 18
Summary of post-translational modifications of AP-101

| | Amino | | Abundance |
|---|---|---|---|
| Position | Acid | PTM | in sample |
| HC1 | Q | pE(Q) pyro-glutamic acid | 99.5% |
| | | formation from N-terminal | |
| | | glutamine | |
| HC258 | M | Methionine oxidation | 2.4% |
| HC321 | N | Asparagine deamidation | 6.9% |
| HC321 | N | Succinimide Formation | 2.5% |
| HC390 | N | Asparagine deamidation | 2.8% |
| HC390 | N | Succinimide Formation | 1.3% |
| HC440 | N | Asparagine deamidation | 1.4% |
| HC453 | K | C-terminal lysine loss | 94% |
| LC17 | M | Methionine oxidation | 1.3% |
| LC168-172 | QSNNK | Succinimide Formation | 6.0% |

1. A recombinant antibody that selectively binds to misfolded and/or aggregated forms of superoxide dismutase (SOD1) for use in the treatment of amyotrophic lateral sclerosis (ALS), wherein the treatment comprises administration of a dose of the antibody of about 2500 mg to a patient in need thereof.

2. The antibody for use according to claim 1, wherein the antibody is administered once every 3 weeks.

3. The antibody for use according to claim 1 or 2, wherein the antibody is administered via intravenous (IV) infusion.

4. The antibody for use according to any of claims 1 to 3, wherein the patient receives a loading dose of the antibody of about 500 mg, followed on the other day by a dose of the antibody of 2500 mg prior to the administration of the antibody once every 3 weeks.

5. The antibody for use according to any one of claims 1 to 4, wherein administration of the antibody will take place once every 3 weeks for at least a 6-month period.

6. The antibody for use according to any one of claims 1 to 5, wherein the antibody is administered at a concentration of about 20 mg/mL.

7. The antibody for use according to claim 6, wherein the antibody is administered over a period of min. 60 and max. 120 minutes.

8. The antibody for use according to any one of claims 1 to 7, wherein the patient suffers from familial amyotrophic lateral sclerosis (fALS).

9. The antibody for use according to any one of claims 1 to 7, wherein the patient suffers from sporadic amyotrophic lateral sclerosis (sALS).

10. The antibody for use according to any one of claims 1 to 9, wherein the patient receives riluzole, edaravone, a combination of sodium phenylbutyrate and tauroursodeoxycholic acid (PB and TUDCA) or Tofersen.

11. The antibody for use according to any one of claims 1 to 10, wherein, if infusion reactions are observed, 500 to 1000 mg acetaminophen, and/or an antihistamine may be administered orally or IV 30 to 60 minutes prior to the start of infusion for subsequent dose administrations.

12. The antibody for use according to any one of claims 1 to 11, wherein the therapeutic effect of the antibody and progression of ALS is monitored by assessing one or more biomarkers.

13. The antibody for use according to claim 12, wherein the biomarkers are selected from the group consisting of phospho-neurofilament heavy chain (pNfH), neurofilament light chain (NfL) and misfolded SOD1 (mSOD1).

14. The antibody for use according to any one of claims 1 to 13, wherein the antibody binds to an epitope of SOD1 within the amino acid sequence 73-GGPKDEERHVGD-84 set forth in SEQ ID NO: 11.

15. The antibody for use according to any one of claims 1 to 14, wherein the antibody is characterized by comprising in its variable region, i.e., binding domain the six CDRs of the variable heavy (VH) and variable light (VL) chain, wherein:
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions.

16. The antibody for use according to any one of claims 1 to 15, wherein the antibody is characterized by comprising in its variable region, i.e., binding domain a VH chain and a VL chain, wherein
(a) the VH chain comprises an amino acid sequence which is at least 90% identical to the amino acid sequence depicted in SEQ ID NO: 1 or 2; and
(b) the VL chain comprises an amino acid sequence which is at least 90% identical to the amino acid sequence depicted in SEQ ID NO: 6 or 7.

17. The antibody for use according to any one of claims 1 to 16, wherein the antibody comprises a human Ig constant region.

18. The antibody for use according to any one of claims 1 to 17, wherein the antibody is a human IgG.

19. The antibody for use according to any one of claims 1 to 18, wherein the antibody a human IgG1.

20. The antibody for use according to any one of claims 1 to 19, wherein the antibody is a human IgG1m3 allotype.

21. The antibody for use according to any one of claims 18 to 20, wherein the antibody comprises a kappa (κ) or lambda (λ) light chain.

22. The antibody for use according to any one of claims 1 to 21, wherein the antibody is AP-101 and a human IgG1m3 allotype.

23. The antibody for use according to claim 22, wherein each heavy chain is comprised of 453 amino acid residues having SEQ ID NO: 12, and each light chain is comprised of 213 amino acid residues having SEQ ID: 13.

24. The antibody for use according to any one of claims 1 to 23, which is produced in CHO-K1 host cells and purified from the cell culture.

25. The antibody for use according to any one of claims 16 to 24, wherein the heavy chain of the antibody is undergoing N-terminal glutaminyl cyclization.

26. The antibody for use according to claim 9 or 10, wherein the heavy chain is undergoing C-terminal lysine clipping.

27. The antibody for use according to any one of claims 16 to 26, which is N-glycosylated.

28. The antibody for use according to any one of claims 1 to 27, wherein the antibody is administered in a formulation of any one of claims 29 to 47.

29. A liquid aqueous formulation of a recombinant antibody as defined in any one of the preceding claims that selectively binds to misfolded and/or aggregated forms of superoxide dismutase (SOD1), wherein the formulation is suitable for intravenous administration and comprises the antibody at a concentration of about 10 to 50 mg/ml in a L-histidine/L-histidine monohydrochloride buffer at a pH of about 6.0±1, wherein the antibody remains stable at 5° C.±2° C. for at least 1 month, at 25° C.±2° C. for at least 1 month, and/or at 40° C.±2° C. for 1 week.

30. The formulation of claim 29, wherein the antibody is characterized by comprising in its variable region, i.e., binding domain the six CDRs of the variable heavy (VH) and variable light (VL) chain, wherein:
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions; and
wherein the antibody comprises a human Ig constant region.

31. The formulation of claim 29 or 30, wherein the antibody is characterized by comprising in its variable region, i.e., binding domain a VH chain and a VL chain, wherein
(a) the VH chain comprises an amino acid sequence which is at least 90% identical to the amino acid sequence depicted in SEQ ID NO: 1 or 2; and
(b) the VL chain comprises an amino acid sequence which is at least 90% identical to the amino acid sequence depicted in SEQ ID NO: 6 or 7.

32. The formulation of any one of claims 29 to 31, wherein the antibody binds to an epitope of SOD1 within the amino acid sequence 73-GGPKDEERHVGD-84 set forth in SEQ ID NO: 11.

33. The formulation of any one of claims 29 to 32, wherein the antibody is a human IgG.

34. The formulation of any one of claims 29 to 33, wherein the antibody a human IgG1.

35. The formulation of any one of claims 29 to 34, wherein the antibody is human IgG1m3 allotype.

36. The formulation of any one of claims 31 to 35, wherein the antibody comprises a kappa (κ) or lambda (λ) light chain.

37. The formulation of claim 35 or 36, wherein each heavy chain is comprised of 453 amino acid residues having SEQ ID NO: 12, and each light chain is comprised of 213 amino acid residues having SEQ ID: 13.

38. The formulation of any one of claims 29 to 37, which is produced in CHO-K1 host cells and purified from the cell culture.

39. The formulation of any one of claims 29 to 38, wherein the antibody is AP-101.

40. The formulation of any one of claims 31 to 39, wherein the antibody is composed of two heavy chains having SEQ ID NO: 12, and two light chains having SEQ ID: 13, optionally wherein in the heavy chain the glutamine at the N-terminus is modified as pyro-glutamic acid and the C-terminal lysine is lost, and wherein the heavy chain is N-glycosylated.

41. The formulation of any one of the claims 29 to 40, wherein the antibody is present at a concentration of 20±5 mg/mL.

42. The formulation of any one of claims 29 to 41, wherein the concentration of L-histidine/L-histidine monohydrochloride is 20±10 mM.

43. The formulation of any one of claims 29 to 42, wherein the formulation further comprises a tonicity modifier, preferably sucrose at a concentration of 8%±1% (w/v).

44. The formulation of any one of claims 29 to 43, wherein the formulation further comprises a surfactant, preferably Polysorbate 80 at concentration of 0.02%±0.01 (w/v).

45. The formulation of any one of claims 29 to 44, wherein the formulation further comprises an antioxidant, preferably L-methionine at a concentration of 0.1%±0.05% (w/v).

46. The formulation of any one of claims 29 to 45 being substantially free of any other additional excipient.

47. The formulation of any one of claims 43 to 46, which comprises or essentially consists of 20 mg/mL antibody in 20 mM L-histidine/L-histidine monohydrochloride buffer, 8% (w/v), sucrose, 0.1% (w/v) L-methionine and 0.02% (w/v) PS 80 at pH 6.0; preferably wherein the antibody is AP-101.

48. A medicament comprising the formulation of any one of claims 29 to 47, preferably wherein the medicament is for use in the treatment of amyotrophic lateral sclerosis (ALS), most preferably for use in a treatment as defined in any one of the preceding claims.

49. A sterile pharmaceutical container comprising the formulation of any one of claims 29 to 47 or the medicament of claim 48, preferably wherein the container is a single-use glass vial to provide 100 mg of the antibody at a 20 mg/mL concentration, optionally wherein the vial contains an approximate 10% volume overfill.

50. A method of treatment ALS, wherein the treatment is performed according to any one of claims 1 to 28, or wherein the treatment comprises administering the formulation of any one of claims 29 to 49 or the medicament of claim 48.

51. Use of the antibody as defined in any one of the preceding claims or the formulation of claims 29 to 49 for the manufacture of a medicament for the treatment of ALS, wherein the treatment is performed according to any one of claims 1 to 28.


SEQUENCE LISTING (13 sequences)

SEQ ID NO: 1 - protein, 123 aa, Homo sapiens
QVQLVQSGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 2 - protein, 123 aa, Homo sapiens
EVQLVESGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 3 - protein, 5 aa, Homo sapiens
AYYIH

SEQ ID NO: 4 - protein, 17 aa, Homo sapiens
VINPSTGTTFYAQNFPD

SEQ ID NO: 5 - protein, 14 aa, Homo sapiens
AISEHGSGSYSPYY

SEQ ID NO: 6 - protein, 107 aa, Homo sapiens
SYELTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 7 - protein, 107 aa, Homo sapiens
SYVLTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 8 - protein, 11 aa, Homo sapiens
SGEALPKKYGY

SEQ ID NO: 9 - protein, 7 aa, Homo sapiens
RDVERPS

SEQ ID NO: 10 - protein, 10 aa, Homo sapiens
LSADSSGTWV

SEQ ID NO: 11 - protein, 11 aa, Homo sapiens
GGPKDEERHVG

SEQ ID NO: 12 - protein, 453 aa, Homo sapiens
QVQLVQSGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, Homo sapiens
SYELTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS